SEQUENCE LISTING (108 sequences)

SEQ ID NO: 1 - DNA, 21 bp, Artificial Sequence
  Synthetic FANA
actgggatacgacaaggatat

SEQ ID NO: 2 - DNA, 21 bp, Artificial Sequence
  Synthetic FANA
tcttggaacagcatagggaca

SEQ ID NO: 3 - DNA, 21 bp, Artificial Sequence
  Synthetic FANA
atatccttgtcgtatcccagt

SEQ ID NO: 4 - DNA, 21 bp, Artificial Sequence
  Synthetic FANA
auauccttgtcgtatcccagu

SEQ ID NO: 5 - DNA, 21 bp, Artificial Sequence
  Synthetic FANA
atgagcgaccttgcgagagaa

SEQ ID NO: 6 - DNA, 21 bp, Artificial Sequence
  Synthetic FANA
gcggcaatgcgttatacggaa

SEQ ID NO: 7 - DNA, 21 bp, Artificial Sequence
  Synthetic FANA
gcggcaatgcgttatacggaa

SEQ ID NO: 8 - DNA, 21 bp, Artificial Sequence
  Synthetic FANA
cgactgctggtggcgaacact

SEQ ID NO: 9 - DNA, 21 bp, Artificial Sequence
  Synthetic FANA
atgagcgaccttgccagagaa

SEQ ID NO: 10 - DNA, 21 bp, Artificial Sequence
  Synthetic FANA
gttttcactgcgctacatgct

SEQ ID NO: 11 - DNA, 21 bp, Artificial Sequence
  Synthetic FANA
gcggcgatgcgttataccgaa

SEQ ID NO: 12 - DNA, 21 bp, Artificial Sequence
  Synthetic FANA
cgcctgctggtggccaacacc

SEQ ID NO: 13 - DNA, 21 bp, Artificial Sequence
  Synthetic FANA
tgaaggacaaggaaattttgg

SEQ ID NO: 14 - DNA, 21 bp, Artificial Sequence
  Synthetic FANA
gaaggacagggaaaatttttg

SEQ ID NO: 15 - DNA, 21 bp, Artificial Sequence
  Synthetic FANA
gattgaagggcaggggaattc

SEQ ID NO: 16 - DNA, 21 bp, Artificial Sequence
  Synthetic FANA
gaagaacgggcgaactttttg

SEQ ID NO: 17 - DNA, 21 bp, Artificial Sequence
  Synthetic FANA
aataaacgcaatgaccaatcc

SEQ ID NO: 18 - DNA, 21 bp, Artificial Sequence
  Synthetic FANA
ataaccttgacgatctgattc

SEQ ID NO: 19 - DNA, 21 bp, Artificial Sequence
  Synthetic FANA
ttctatcagggtaaatcgctc

SEQ ID NO: 20 - DNA, 21 bp, Artificial Sequence
  Synthetic FANA
gtcgttagatgaacgatatat

SEQ ID NO: 21 - DNA, 21 bp, Artificial Sequence
  Synthetic FANA
ccaagatctaattgcttggcg

SEQ ID NO: 22 - DNA, 21 bp, Artificial Sequence
  Synthetic FANA
gctgacttagtacttgccgcc

SEQ ID NO: 23 - DNA, 21 bp, Artificial Sequence
  Synthetic FANA
ctccgatagcggttatttgcg

SEQ ID NO: 24 - DNA, 21 bp, Artificial Sequence
  Synthetic FANA
acuuucctaaaacaagaggcc

SEQ ID NO: 25 - DNA, 21 bp, Artificial Sequence
  Synthetic FANA
guuccauaggtaatccugaau

SEQ ID NO: 26 - DNA, 21 bp, Artificial Sequence
  Synthetic FANA
aagagugcagcttaucguugu

SEQ ID NO: 27 - DNA, 21 bp, Artificial Sequence
  Synthetic FANA
gcgtagtaaccagtaacaacg

SEQ ID NO: 28 - DNA, 21 bp, Artificial Sequence
  Synthetic FANA
ggcctcttgttttaggaaagt

SEQ ID NO: 29 - DNA, 21 bp, Artificial Sequence
  Synthetic FANA
attcaggattacctatggaac

SEQ ID NO: 30 - DNA, 21 bp, Artificial Sequence
  Synthetic FANA
acaacgataagctgcactctt

SEQ ID NO: 31 - DNA, 22 bp, Artificial Sequence
  Synthetic FANA
gttcttacgttcttgcatctga

SEQ ID NO: 32 - DNA, 21 bp, Artificial Sequence
  Synthetic FANA
cguuguuactggttacuacgc

SEQ ID NO: 33 - DNA, 21 bp, Artificial Sequence
  Synthetic FANA
acuuucctaaaacaagaggcc

SEQ ID NO: 34 - DNA, 21 bp, Artificial Sequence
  Synthetic FANA
guuccauaggtaatccugaau

SEQ ID NO: 35 - DNA, 21 bp, Artificial Sequence
  Synthetic FANA
aagagugcagcttaucguugu

SEQ ID NO: 36 - DNA, 22 bp, Artificial Sequence
  Synthetic FANA
ucagaugcaagaacguaagaac

SEQ ID NO: 37 - DNA, 21 bp, Artificial Sequence
  Synthetic FANA
tgatgggcaaggtaactttgg

SEQ ID NO: 38 - DNA, 21 bp, Artificial Sequence
  Synthetic FANA
tggtatggcaaccaatattcc

SEQ ID NO: 39 - DNA, 21 bp, Artificial Sequence
  Synthetic FANA
ggtgttgcagttggtatggca

SEQ ID NO: 40 - DNA, 21 bp, Artificial Sequence
  Synthetic FANA
ccagggccggattttccaacg

SEQ ID NO: 41 - DNA, 21 bp, Artificial Sequence
  Synthetic FANA
cattcaagaaggacaaggaaa

SEQ ID NO: 42 - DNA, 21 bp, Artificial Sequence
  Synthetic FANA
cgataagctgcactcttatca

SEQ ID NO: 43 - DNA, 21 bp, Artificial Sequence
  Synthetic FANA
ggtgcttattcttttgttgca

SEQ ID NO: 44 - DNA, 21 bp, Artificial Sequence
  Synthetic FANA
tagcctcaatgtttcctttcc

SEQ ID NO: 45 - DNA, 21 bp, Artificial Sequence
  Synthetic FANA
ggatatgcaaagcattcagga

SEQ ID NO: 46 - DNA, 21 bp, Artificial Sequence
  Synthetic FANA
caattaatgggaaaatttccc

SEQ ID NO: 47 - DNA, 22 bp, Artificial Sequence
  Synthetic FANA
gtttgtaggaacataaatttcc

SEQ ID NO: 48 - DNA, 22 bp, Artificial Sequence
  Synthetic FANA
caacaattgtatcatataaagg

SEQ ID NO: 49 - DNA, 21 bp, Artificial Sequence
  Synthetic FANA
ctttaataaaataatattcga

SEQ ID NO: 50 - DNA, 21 bp, Artificial Sequence
  Synthetic FANA
gctgcggcaatgcgttatact

SEQ ID NO: 51 - DNA, 22 bp, Artificial Sequence
  Synthetic FANA
ccaacagcaggtattatttatg

SEQ ID NO: 52 - DNA, 21 bp, Artificial Sequence
  Synthetic FANA
ccttttgtaaaatgtgcacga

SEQ ID NO: 53 - DNA, 21 bp, Artificial Sequence
  Synthetic FANA
catgaaatgaataatttatgg

SEQ ID NO: 54 - DNA, 21 bp, Artificial Sequence
  Synthetic FANA
attagtagatggacaaggtaa

SEQ ID NO: 55 - DNA, 21 bp, Artificial Sequence
  Synthetic FANA
ccaacctggacacatttttcc

SEQ ID NO: 56 - DNA, 21 bp, Artificial Sequence
  Synthetic FANA
gaggagtattaatacgaggag

SEQ ID NO: 57 - DNA, 21 bp, Artificial Sequence
  Synthetic FANA
gaggaggtcatactgaagctg

SEQ ID NO: 58 - DNA, 21 bp, Artificial Sequence
  Synthetic FANA
gaaggagatattatattatct

SEQ ID NO: 59 - DNA, 21 bp, Artificial Sequence
  Synthetic FANA
gaaggagatattatattatct

SEQ ID NO: 60 - DNA, 22 bp, Artificial Sequence
  Synthetic FANA
ctatccttgtgaataatgaggc

SEQ ID NO: 61 - DNA, 22 bp, Artificial Sequence
  Synthetic FANA
gcttccgaagcagtttcaataa

SEQ ID NO: 62 - DNA, 21 bp, Artificial Sequence
  Synthetic FANA
ggctggacaagcgtttaatcg

SEQ ID NO: 63 - DNA, 21 bp, Artificial Sequence
  Synthetic FANA
tattcttgcttatacaaagtg

SEQ ID NO: 64 - DNA, 22 bp, Artificial Sequence
  Synthetic FANA
gcatcattattcactaggatag

SEQ ID NO: 65 - DNA, 21 bp, Artificial Sequence
  Synthetic FANA
tatggaaacagcctcagatgc

SEQ ID NO: 66 - DNA, 21 bp, Artificial Sequence
  Synthetic FANA
cgattaaaagcttgtccagcc

SEQ ID NO: 67 - DNA, 21 bp, Artificial Sequence
  Synthetic FANA
cattttgtatatgcgaggaca

SEQ ID NO: 68 - DNA, 21 bp, Artificial Sequence
  Synthetic FANA
ttgtccagccatatcaatagc

SEQ ID NO: 69 - DNA, 21 bp, Artificial Sequence
  Synthetic FANA
tagttccgatttgtatgcatc

SEQ ID NO: 70 - DNA, 21 bp, Artificial Sequence
  Synthetic FANA
tgtatatgcgaggacatcgtc

SEQ ID NO: 71 - DNA, 21 bp, Artificial Sequence
  Synthetic FANA
cggcacagcaattagaacgtc

SEQ ID NO: 72 - DNA, 20 bp, Artificial Sequence
  Synthetic FANA
ggaagggatattcgtgtagg

SEQ ID NO: 73 - DNA, 22 bp, Artificial Sequence
  Synthetic FANA
cggagaggatgttagtgcttgt

SEQ ID NO: 74 - DNA, 21 bp, Artificial Sequence
  Synthetic FANA
cctgatttagcacgctctgag

SEQ ID NO: 75 - DNA, 21 bp, Artificial Sequence
  Synthetic FANA
tattacgatagatcctcgacc

SEQ ID NO: 76 - DNA, 21 bp, Artificial Sequence
  Synthetic FANA
caaatcaggtatagcacggcc

SEQ ID NO: 77 - DNA, 21 bp, Artificial Sequence
  Synthetic FANA
gggaatattcgtcgccatacc

SEQ ID NO: 78 - DNA, 20 bp, Artificial Sequence
  Synthetic primer
actaacatcatgccgcttcc

SEQ ID NO: 79 - DNA, 20 bp, Artificial Sequence
  Synthetic primer
attgacgcacatcgcttaca

SEQ ID NO: 80 - DNA, 20 bp, Artificial Sequence
  Synthetic primer
agggctttactcaaaattgg

SEQ ID NO: 81 - DNA, 22 bp, Artificial Sequence
  Synthetic primer
caccaacgtatggagtgatagg

SEQ ID NO: 82 - DNA, 20 bp, Artificial Sequence
  Synthetic primer
attccaatcgaggcattgag

SEQ ID NO: 83 - DNA, 20 bp, Artificial Sequence
  Synthetic primer
tcagagcgtgctaaatcagg

SEQ ID NO: 84 - DNA, 20 bp, Artificial Sequence
  Synthetic primer
tcgagcgcgtatgcaatacg

SEQ ID NO: 85 - DNA, 24 bp, Artificial Sequence
  Synthetic primer
gcgttatcccgtagaaaaaggtag

SEQ ID NO: 86 - DNA, 18 bp, Artificial Sequence
  Synthetic primer
agacgggtgagtaacgcg

SEQ ID NO: 87 - DNA, 23 bp, Artificial Sequence
  Synthetic primer
gctctcaaagatcggtttgacgg

SEQ ID NO: 88 - DNA, 21 bp, Artificial Sequence
  Synthetic primer
gctgccacgaacgttaccttc

SEQ ID NO: 89 - DNA, 23 bp, Artificial Sequence
  Synthetic primer
ttactgaccatcactctggacgc

SEQ ID NO: 90 - DNA, 22 bp, Artificial Sequence
  Synthetic primer
gtatgccacgtcgcattccaga

SEQ ID NO: 91 - DNA, 22 bp, Artificial Sequence
  Synthetic primer
gccaaaactgctaagggcattc

SEQ ID NO: 92 - DNA, 19 bp, Artificial Sequence
  Synthetic primer
atccagatgcttacgctgg

SEQ ID NO: 93 - DNA, 2196 bp, Unknown
  Candidatus Liberibacter asiaticus
atgaaaaaacgtcaattgattccaatcgaggcattgagtatcgaacaagctagaaaagaattatcgtctcttgaacaagagatatcatatcatgatgattgttattatcagtattcaaaacctataatcttagattctgaatatgacgcattaaaaagacgttgtgatgctattgcacatgcttttcctgatttagcacgctctgaggatcctaatgggcctctgaataaaatagggggagaaatgaacctctccttatttaaagtgactcattctatcccaacattatcattagaaaagatttggaatatcgaagatcttcgtaagttcatcactagaatttatcgtttttggcaaaaaaaagatgataacagcattttttttactctggagcctaaaattgatgggacaatgattactatacgatatgaaaaaggaaagtttgtttatgctgctttgcgtggtgacggtcatagcggagaggatgttagtgcttgtattcgtgctattccgacaattcctcgtgttttatcattgaatattccagaaattatagaagtgcgtggtgaagtttacatctcaaaaaatgattttcttgcgcttaatgatgaaatgattgcagtcggccgaaaaccctatgctaatcctagaaacgctgcttctggcatattacgtcgtttaaatcctacggatattactcggtatttgaatttttttgtgcatggattaggaaaaacatctgagatattcgcaaaaggtcagtatgaaatgctacaaaaaatgcgttgtctaggctttcctgttaataacggagttcgacaagcgaatacttttcacggtatcgtatcctattataatgaaatgaaatcaatacgttctactcttccatatgatatagatggattggtatataaggtcgatgaattttctttacaaaaacaattaggagaacgagctcgaagcccacgttggatgatatcgcataaatttgcagagaaacaagcatctacgcgtcttttagatattgatattcaaattggtcgtactggtattctcacgccggtagctcgtcttgaacctgttaacataggaggtgctttaatcactaatgcaacattgcataatgaagattacatcaaaggattggatgcatctggtaaagtaatgcgtggaggaagggatattcgtgtaggggatagggtcttggtcaaaagagccggagaggtgattcctaaagttgtggatattattgtcaatgaaagacatcctgatgctcaaccgttttcttttccttctttttgtcctatctgtcagagtcgagtagtgcgtgatttaaatccgaaaacaggaaaacttgttgctgcccatcgttgtacgggaggacttgcgtgctcggcacagcaattagaacgtcttaagcattttgtctcgcgagatgctttcaatatagaaggtttaggaaaacaacaattggatttcttctttaaatcagaagatcctgctttttctattaggattcctgccgatatttttacattgcaacgtcgtcaacaaacttctactacaaagattgagaatattctaggttttggcgatgttagtgtcaccaatctttacgattctattaataaaagacgcaatatttctttggaaagatttatattttccttaggtattcggcatgttggcgcagaaattgccagatctttggcgaaatattatctctcatatcaaaattttgcacaagaaatcaacaacattattgcacataacaatgatgattggctgtctttaataagagttcctctagtaggagatataatagcgcaggctattgtagaattttatcagaatcctaggaatatttgtgctgtagaagcactgttaaaagaagtatctccttctattgggcgtcacgaaaaaaatgtcagctctgagatcgaaaataagaaattagtatttaccggaaccctgcagaaaattcaacgccataaagcacaagaatatgtgactcagttaggtgctgttgtttctgctattctttctcgtaaaactgatattattattgttggtgataatcctggttccaagttagagaaggcacaacaactaggtgttaaaataatgaacgaagagcaatttttatttctattacaacagtataatacaacgttgcgtatccacgacgatgactaa

SEQ ID NO: 94 - DNA, 3080 bp, Diaphorina citri
caaagtatagaaaatgcaagacaatatagtaccagtttcgatagtaaaagagctagaagattcttatctctcctacgcaatgagtgtaatcataagccgagctatacctgacgtgcgagatggatttaaacctgtacataggcgtatattatatgcaatgtcaagagctgggttcgatgcaggtaagccatataaaaaggcagctcgtatagttggagatgtaatgggaaaatatcacccacatggtgatgcagctatttacgattccttggtcaggatggctcaagatttttctcttcttttaccacttattgatgggcaaggtaactttggttcgatagatggagatccgccagcttcaatgcgatatacagaagcaaggctccacagggtgtcgcattttttactgaatgatattgatgaagatacagttgattttagggcaaactacgacggaaatgaaacggagcctgtcgtattgccggcagaatttcctaatctgttggtaaacggtggaagtggtgttgcagttggtatggcaaccaatattccttctcataatcttggtgagataattgatgcttgcatgttatatatagataaccctgaagttactctagatgagttgcttgaagtaataccagggccggattttccaacgggaggaataattcttggcagatctggaataagatcagcgtttgctaaaggccgaggttcaattgtcgttcagggtaaaactcatatagaaaatctgccacaagatagacaagcaatagttattgatgaaataccttaccaagtaaataaagtaaaattaattgagaaaataggcgagctcgcaaaagaaaaaagaattgagggcataacagagattagggatgagtctgataagtctggtattagagtagtaattgatctcaaaaaaaacactgaagcagattttatattgaatcaaatactaggacttactcctctgagaagtagctttagtgttaatactttagttctgaacaacaacaggcctgctttgatgtcattgaaagaaatcatagctgcttttattgactttagaaaagaagtactaatcaggagaacagagtttcgtctaaaaaagacgagagaaaaagctcatatatacatagggctctacattgcggtcctcagcatagatgaagtgataaaaatcatccgtggtgcaaaagatcctgaagaagcaagcagagagcttttaaacaaggaatggaaaacctcagctgaaataaacacgattattgaattaatctcagacagtatgagctttttgaaagatggagtgtatagattaactgagctgcaaacaaaagctattcttgatatgaaattgcaacgcttaacaggccttgaaaaaagcaaattagaaaatgagttaaattcaatgcttagcctgataaaagaatatatcgcttttcttggctcagaagagaaattgatgcaagaaataaaaaacaatttacaagaaataaagaatagatttgccgtacctcgaagaacttcaatagaagaatcagacatggacattgaagctgaagatctaattccacaagaagatatggtagtgaccgtaactatgaatggttacatcaaacgtgtgaagctctctcactatagaacccagcatcgtggtggaaaaggaaagctaggacagggattaaaagaggaggacgtaatcacaaaattatttgttggaaatactcatactagccttttattcttttctaatatcggtcgagtttacagattaaaagtttacaaactgcctcttgcagagccaactgcacgtggaagagcgcttgttaatatattcccgcttagcgatggtgaaactataactaacatcatgccgcttccaagtgagaatgacgaaaatcaaaatatagtttttgctactgctcatgggaacataaggcgtaactccttagcggattttcactatattccaagcaatggaaaaatagcaataaagctcgatgaaggggataaattagtgtcagttaaagtatgtagcgaaattgatcacgttctactttcaacaacgcttggaaaaagcattagatttgttgtaagcgatgtgcgtcaatttaagagtcgcaattcagatggagtaagaggtatcaaacttgcaaaaaatgacagcgtgatatccacgaccatactaaacggcataggtattgcaacagaaacaaaagaactttacttaagagttccgcttgcaaaaaggatggaagctgcaaccaataactcaattgattctaaatcagaaaaaactttgaatgatttaggaataaagaacgaattattctcaaaactcgcacttaatgaggaattcatattaactattactgaaaatggctttggtaaaagaacttctgcatatgaatatagggtaacaggtagaggtggtgttggtattacaaatatccttactactagtagaaatggcaatgttgttgctagttttccagttgagcaggatgacaacataatgcttattacagataaaggaaagttaattcgcatttcggttaatgaaattagaattgcaggacgcagcactcagggagttactctgtttaaaacagaaaataaagaaaaggtggtatcggtagcaaaaattgaagatcctagctccactgaagagaatatgcctgaggttgaaagtgctacttcttcttaattatgtagcaaaggctaagcaaacagtgcaaatttctattgattaatctataatatttaaattaaaatatatgtctatgttcaagtagagattgatggataagaagttaactaaaaataaaaaatcattgacgaatgagaagaataaaaaaacatctcttgttaaagattttgccaaaaataagagtagaagagattttatcacattaactacgtgtgccatggcaggtataggagctgcaagcggtttttggccattggttaagtctatgaatccttctgctgaggttttagcaatgtctacggttgaggttaatctatctgacattcaagaaggacaaggaaaaaaagtaaaatggcaaggt

SEQ ID NO: 95 - DNA, 1680 bp, Unknown
  Candidatus Liberibacter asiaticus
acctaaaattgatcctatagatcctgatgtactccaccaaaaatttcaagaacactttacacaatcattattacagaaatatttctatgaatcacttacattctaattatccgatatggcatccttttgttcaacatttttgtaatcctacttttaaaaagattattaagacagaaggatcttatcttattgatgaaaatcacgataagattattgatgccatttcttcttggtgggttattacccatggacatcgtcatcctataattatgaatgcaatacgatctgcatctgaaaactatgatcaaataattttttccgaatatactcacgaacccgctgaacaacttgcaagattactcattaaaattgcacctctagggttacaatacgtttttttttctgatagtggctctacttctatagaagtagcactcaaaatggccctagggttttttaataataataacatgccacgtaacaaaattgtcgtcatggaacatggataccatggagatacaattggatctatgtcagttagtatgcgtggagcatttaataattcttatgaacctctactgttcgatgtagaaaccattccttttccacatcataatgaagaacaaaaaacccttgatgatcttgaaaaacactgtagtacccaaaaaatagctgctttcttggtagaaccattaatacttggtgcgggcggtatgcaaacctatcgtccatacattctaaaagaatttatgcgtataacaaaaaaatatgatacacttctaattgcagatgaagtcatgactggatggggaaggacagggaaaatttttgcttgtaatcatgcagatattactcctgatattctttgtttatctaaaggaattacaggaggtgcattaccattagcagctacactttgctctgaagaaatttttgattctcacttatcttctgataagaagaaaacattttttcattccagctcgtatactgctaatcctattgcttgtgcggcagcacttgctaaccttcaaatatgggaaaaagaacccgtaatagagcgtataaaaaatttagaaagaatgcatgctgataggttgtcattacttaaatcgaacaaaaaatttgtcaatataaggcaaatgggcacaattgtagcgctagattttaatgttcaaaacaaaggttatttttttgataaaaatgctaaattaaaaaatttttttcgcgaaaaaaagattcttattcgccctttaggacatataatgtatcttatgcccccatattgtataactgctactgatcttgatcacatatataatacaattaacgaagctgttgattttttggagttataattgataaaaagttctagctgcattctcggattcggacatgctgttcctaaccaatgcattcataattctataattgaaagacacctaaatcttaagctaggtgttatagaacaaaaaactggtattaaatatcgatattgggctgaaaaacacgaaacactaactgatattgcaatacaagcaggagatatagctttacgaaatgctaatatcaaaaaggatagcatttctttaacacttcttgctacttcaacacctgatcacttattaccaccatcttctcctctcatcactcatcgcttgggacttatgcaatctggag

SEQ ID NO: 96 - DNA, 1120 bp, Unknown
  Candidatus Profftella armatura
atgaatcaatttgctaaagaagtaatttctattttacttgaagaggaaatgtgtaaaagttatcttgattatgcgatgagtgtaattgttgggagagcattaccagatgtgcgggatggattaaaaccagttcatagacgtgtattatttgctatgcatgaaatgaataatttatggaatcgtccttttgtaaaatgtgcacgagttgttggggaaactatgggaaaataccatcctcatggtgatgtatcaatttatgatgctttagtaagaatggcacaatcattttctttacgttgcacattagtagatggacaaggtaattttggttctatagatggggatagtgctgcggcaatgcgttatactgaatgtcgtttaaataaaatttctaatgaattacttattgatttagataaagaaactattaattttatttcaaattatgatggaaaagaaaaagaaccatctgtattaccaacacgtattccaaatttactaataaatggttcttctggtattgctgttggtatggcgactaacatacctcctcataatttaacagaagttattgatggtgtattatatgttttacataatccagaatgctcaattaatgatttaatcaagataattccagctcctgattttccaacagcaggtattatttatggattatctagtgttcatgatggctattatactggaaaaggtcgtgtaataatacgagccaaaactcatatagaagaatttaatagagaaaatcgcacagcaattattattgatgaacttccatatcaagttaataaaaaatcattattagaaaaaatttctcaattagttaaggaaaaaaaattagaatgtatttctaatttacgagatgaatcagataaatctgggatgcgtatagttattgaattgaagagaaatgaaataccagaaatagtattaaataaattatataaacaaactcagttacaaaatacctttggtatgaatatgttagcattagttaatggacagcctaaattattaaacctaaaagaaatactttattattttattttacatcgtaaagaagtaataaaacgaaggactatttttgaattacgtaaagcgc

SEQ ID NO: 97 - DNA, 700 bp, Unknown
  Candidatus Profftella armatura
atgaatcaatttgctaaagaagtaatttctattttacttgaagaggaaatgtgtaaaagttatcttgattatgcgatgagtgtaattgttgggagagcattaccagatgtgcgggatggattaaaaccagttcatagacgtgtattatttgctatgcatgaaatgaataatttatggaatcgtccttttgtaaaatgtgcacgagttgttggggaaactatgggaaaataccatcctcatggtgatgtatcaatttatgatgctttagtaagaatggcacaatcattttctttacgttgcacattagtagatggacaaggtaattttggttctatagatggggatagtgctgcggcaatgcgttatactgaatgtcgtttaaataaaatttctaatgaattacttattgatttagataaagaaactattaattttatttcaaattatgatggaaaagaaaaagaaccatctgtattaccaacacgtattccaaatttactaataaatggttcttctggtattgctgttggtatggcgactaacatacctcctcataatttaacagaagttattgatggtgtattatatgttttacataatccagaatgctcaattaatgatttaatcaagataattccagctcctgattttccaacagcaggtattatttatggattatctagtgttcatgatggctatt

SEQ ID NO: 98 - DNA, 1386 bp, Drosophila simulans
atgctagatgaaatgcgtgaggaatgtggggtatttggcataagttgcaaccaaagtgctgcttttaactctatacttgctctccacgctttgcagcatagaggtcaagagtcttttggcgtagtaaccagtaacaacgataagctgcactcttatcactttcaaggtcaagtgagcagcgtatttgatgatatagatgaaataaagaaatccttacctggagattgtgcaataggtcatgttcgatactcaacaagtggtaataagtttggagtgcagcccatgtttagcaaaagtggcaaatttggggatttcgccatagcacataatggtaatttaattaatatttctccgatacgcgaacaattgattaaacaagagtgcgtttttcagtcagatattgatacagaagtagtagtgcatctgacagcaagcggtgaaaaagatagctttttagagagttttatatatgcattaaagcaaatacaaggtgcttattcttttgttgcaatcaatcaagaagtagttattggagtacgtgattcatctggaatcaggcctcttgttttaggaaagttaaacggttcttacgttcttgcatctgaaacttgtgctcttgatataataaatgcagagtttgttagagaaatagaaccaggcgaattagttaccatcagttcgggtagtaagctagcctcaatgtttcctttcccacagcaaaaatcaagtttttgtatttttgaatacgtttatttttcaagaccagacagcataatggagaataggtctatatacgatacaagaaaagaaataggaagaatacttgcagaagagagcccaccaaaaaacaatgtagatatggttgtaccaatacctgattctggaatacctgcagctattggatatgcaaagcattcaggattacctatggaactgggaataattcgtaatcattacataggaagaacttttatacaaccaaccgctgaagtacgtaaagttagaataaaattaaaattcaatgctaataagcacactttgaaaggtaaaaacataattttaatagatgatagtatagtgcgtggtagtacgttaacaaatataatagttatgctaaaggatgcaggagtaaaagagattcacctcaaaatttcaagcccaccaattaagcattcttgtttttatggaatagatacgccagagtgcaaagatttaattgctgcaaataaatctgtagaggaaattaaggaagttataggagtagatagcctagccttcttgagtattgatggattatatcaggctgttaaaggagaagtgcgtaacaatgctacaccacaatattgtgatgcttgtttcactggtgattatccaattggcaaatag

SEQ ID NO: 99 - DNA, 1679 bp, Unknown
  Candidatus Liberibacter
cctaaaattgatcctatggatcctgatatacttcatcagaattttcacaaaaattttgcacaatcactattaaagaaaaatgtttatgaataattcaaactataagtttccgatatggcatccttttgttcaacattttttcaatcctacttttaacaagattattaagacacaaggaacttatcttattgatgaagatcacaataagattatcgatgcaatttcttcttggtgggttattacgcatgggcattgttatccaaaaatcatgcgtgccatacgatctgcatctaaaaattatgatcaaataattttcgctgaatatacccatgaacccgctgaaaaacttgcaaaatcactgattaaaattgtacccccagggttacaatatgtttttttctctgatagtggttctacttccgtagaagtagcactcaaaatggcgctaggttttttttataataataatatagaacgtcataaaatcgttgtcatggaacatggatatcatggtgatacaatcggagctatgtcagttggaatgcgtggagcatttaataatccttatcaacctctattatttgatgtagaaaaaattccgtttccatatcctcattcagaacaagaaactattgattgtcttgaacaacactgtcgcaccaaaaagattgctgctttactgatagaaccactaatacttggtgcaggaggtatgaaaacctatcgtccatatcttctaaaagaatttatacggataacaaaaaaatatgatacccttctcattgcagatgaagtgatgactggatggggaagaacgggcgaactttttgcttgtaatcatgcaaatattaccccagacatactttgtttatctaagggaataacaggaggtgcattaccactagcggctacactgtgctctgaagaaatttttgattctcatgtatcttctgaccctagaaaaacattttttcattctagttcttatactgctaatcctattgcttgtgcagcagcacttgctaatcttaaaatatggcaaaaagaaccagtaatggaacgcattaaaaatttagaaaaaatgcatgctgataagttgcttttattccaatcaaataaaaaatttattaatatcagacaaataggaacaattgtagcgcttgatttaaatgttcaaaataaaggttatttttttaaaaaaaatgcagaattaaaaaaatttttccgcgaaaaaaatattcttatccgccctttagggaatgtaatatacctcatgcctccttactgtataacatcaagtgatattgatcatatatataatacaattaatgaaactgttgattttttggagttatagttgatgaaaagttctagttgtattcttggattcggacattctgttcctagccaatgcattcacaattcgataattgaaagacacctaaatcttaaatctggaattatagaaaaaaaaactggtattaaatatcgatattgggcaggaaaaaacgaaacgctaactgatattgcaatacaagcaggagatatagccttacgcaacgctaatattaacaaggatagcattgctttaacacttcttgccacttcaactcctgatcacttattaccaccatcatctcctgtagttacccatcgccttggacttttaaaatccggag

SEQ ID NO: 100 - DNA, 1515 bp, Unknown
  Candidatus Liberibacter asiaticus
atgattaatacagctcaggatatcattttatctattccaaaagaagattcctctcaatatcgtgaatctcccaataacattgaagcagaacaagctcttttaggagctatccttgtgaataatgaggctttttatcgagtatcagatttcttaaaaccgttgcattttttcgaagcgattcaccagaaaattttcgaaataatgggtaagttggttcacatgggaaaaacagctaatcccgttacggttaaaactttcttatctgatcaagatatgttaggagagctaacagtaccacagtatttagcgcgtctagcttccgaagcagtttcaataataaataccgaagattatggaagaataatatatggattagcgctgcgccgtactctcatcactattggtgaagaaatggtgaatgccgcttatgaagcgtctcttgataaatccccttcaatccaaattgaagaagcagaacgaaaactatttcatttagcagaaaatggacgatacgatgggggattccatacattttctgatgcaatgacagtcgcaattgatatggctggacaagcgtttaatcgagatggaagactcgccggagtttctacaggaattcaaacgcttgataaacaaatgggaggattacaacgttccgatctcataattattgcgggacgcccaggtatgggaaaaacatcactggcaacgaatattgcctataatgttgcagatgcatataaagcagaactacaaacagatggatcttataaaactattaatggtggcattgttggattctattctttggaaatgtcttctgaacaactcgctacccgcattatttctgaacaaacagaagttccttcttcaaagatacgcagaggagaattgacgcgacctgactacgaaaaaattgttgcttgttctcaggtaatgcaaaaattaccactatacattgatcaaactggtggtatttccatgagtcaattagcgacacgtgcacgtcgattaaaacgtcaacgaggacttgatcttcttattgtagactatatacaactaatgactacttctaaaaaaattgaagaaaatcgtgttctggaaatcacaggcattaccatggcattaaaagctctggcaaaggaacttaatatacctatcattgcgctttcacaactttctcgacaagtagagaatcgtgataataaacgccctcaactttctgatttacgtgaatctggttccattgaacaagatgcagacgtagtactattcgtcatccgcgatgaatattatatcagaaacaaagaacctacaaacaaagatgatattcttgcttatacaaagtggcaggaagatcttaaaagagtaaaaggcattgccgatattattatagccaaacaacgtcatggaccaacaggaacggttacccttgcttttcaagcagaatttacacgcttttccgcattatctgattcgtcttatcaaactggtgaacattttgattctgcataa

SEQ ID NO: 101 - DNA, 1152 bp, Unknown
  Candidatus Carsonella ruddii
atgattaagaatatttcttctattaattcagaatttattataataaattatatttataatgatttttatttttctaaaaaaatattagaaattttagaagaagatgatttctttttcgacacaacaaaaaaattttttttacaaaaacaattttttttaaaattaaaatttttggatgaaaaattagttttttctaatcttaaaaatttattagaaaaatctaaaaaaagaaaaatgttaaaaatattatttaacataagttttgatttaatatattataacaatattgaagaaaatatatttgataaaataatttttttgcaaagttttaaaaaaagtaattttttttttaaaaaaattgattataaaaaaatactaaaaaattattttttagagaatgaaaaaaatttttttacaggatacagtaaattagataatattataaatggtttacaaaaaggaaatttaataatcttagcaggaagaccttcaataggaaaaacttctttttcattaaatattgttaagaatttaatattattcaaaaaaaaaataattttcttttctttagaaatgacagtaagtcaacttttaataagattaatatctattatactagaaattaatcacgataattttgtaaaaaataattttaataatatagatataaaaaaaatttcttttttcataaaaaacataaattttgatgaattaataataaaagactgttcaactctatcatctaatgatatagaatcgcaactagagtattataaaaagaataatattaatattgaaataataatcattgattatattcaattaatgaaatctgaattttattcaaataatagagttttagaaatatccgatatttctagatcattaaaattaattgcaaaacattttaattgtgttgtaattgcgctatctcaacttaatagattagtagaatatagagttgaaaaaactcctgttttatctgatttaagagattctggttcaattgaacaagatgcagatatagttatttttttacacaaaaaaaaattaaactttgttgatattttagtagctaaaaacagaaatggttctttaggaataataaattttatttttaaaaatgaatatactaaatttttacaaatttaa

SEQ ID NO: 102 - DNA, 759 bp, Unknown
  Candidatus Liberibacter solanacearum
ttaaataaaaatatctcctgtaaaaattttacgaaaacaaccttttttttcttccaataataaatatccaaaatcatcaactccaacaaaatgacctgaaatagaaaattggggaagtttaatcgtgataggattacctattccgtaagcaaaacaacgccaaagtttcatgatttcttctcgtcctgtatcttttttccaaatagctaacatttttgcaatttcttggaaaaaaattggtaaaatactctttaaatctatacatcctccctcttttctcagagaagtaacaggataagaagtatctttgggacaatactcaacgttcaaccctattccaataacaattgcttgtaaaccactttttaaatctaaagtctcaattaaaattccagcaatcttacgttgaaaaactaacaaatcatttggccattttattttgacatctacctcagcaggaagaatagatgctataattgagtgcatagcaacagaaactgcaaaagacaagagcgttagggaatccttcgatatagaatcaatcaatagtaacgaagcgtatagattccctttatcagaaacccattttttccctccacgacctcgaccagatgtttgacgtgatgcaattatccataaatttcctaaatcgccagataatgcacgtttcatacattcatcgtttgtagaaacgatagtatcaaaaaattcataccgaaaattataaagttttttacattcagaaagatcagataacat

SEQ ID NO: 103 - DNA, 759 bp, Unknown
  Candidatus Liberibacter asiaticus
atgttttctaatcttagtgaatgtcaaaatccttatagttttcgatatgaattctttgatactatttcttctactaatgatgaatgtatgaaacgtgcgttatctggagatttgggaaatttgtggatcgttgcgtcttgtcaaacggctggtcgaggtcgtagggataataaatggatttctgataagggcaatctctatgcctcgttgttattgattgattctatatcaaaagattccctaacgctcttatcatttgctattgctgttgctatgcgttcagttatagcatctacacttcctgttggaacagatgttaaaataaaatggcccaatgatatactagtctttcaacgtaaaattgctggcatattaattgagactctaaatttaaaaaatggtttacaagcagttgtcgttggaataggattgaacgtaaagcattgtcctgtagacaccccttatcctgttacatctttgcagagagaaggaggatgcatcgatttaaaggatattttttcccttttatttcaagatgttgcgagggtattagacctttggaaaaaagatacgggacgagaagaaattatgaatctctggcgttgttttgcgtgtggtataggtgatcttattacgattaaactatcatatggttccattttaggtcgttttgttggagtcgacgatttcggatatctattattagaagaaaaaaaaggatgtgttcgtcagatttttacaggagatatttttacttag

SEQ ID NO: 104 - DNA, 633 bp, Unknown
  Candidatus Liberibacter asiaticus
atgaaacagaaaaagtcgttagatgaacgatatattgaagatgtcattcaaagttttcaaaaaggagaaatggttatcgtcacggatgcagatgaccgcgagaatgaagctgacttagtacttgccgccatacattgcacatccgaaaaaatggcatttatcatccgccatacttgtggaatcgtttgcactcccatgccctttcacactgcacataagcttaaacttaatcctatggtattagaaaatgaatccgttcacaaaactgcttttaccgtctccgttgattctaaacatggaatcactactggcatatctgccgatgacagagcatataccatcaaaaaccttgctaatccacacagcattgcggataattttgtccgcccaggacatatcttccctcttatatcccgcgatggaggagtactagttcgccctggtcatacagaagcatcagtcgatttatgtaaaatcacaggactacctccgatagcggttatttgcgaattagttaacgatgatggaacaatcaaaaaaggaaaacaagtgattgaattttcaaagaaatatgacttgaaaatcatttcagtccaagatctaattgcttggcgaaagaaaaaagaaatataa

SEQ ID NO: 105 - DNA, 1116 bp, Unknown
  Candidatus Profftella armatura
atgtacaataaatttatttcttctactaaagaaattatttctgaattaaagattggaaatatgattattttagtagattcagaaaatcgtgaaaatgaaggagatattatattatcttctgattttgtaacaccaaatgctattaattttatgtctaaatatgcaagaggtttaatttgcatgacattaacagaaaaacattgtattcagctcaaattagatatgatgacaaaaaaaaataattcttcttttggtactaattttaccgtttcaattgaagcggcaaatggagtcacaacgggaatttcggcatcagatcgagcacatacaattaaagttgcttcatcaaaaaaagctaaaccttctgatatcgtccaacctggacacatttttcctctacaagctaaaaaaggaggagtattaatacgaggaggtcatactgaagctggttgtgatttaacaaaattagctggactaacaccatctgcagtaatttgtgaaattttaaatgatgatggaactatggcacgtttaccagatctaataaaattctctaaactacataatattaaaattggaactatttctaatttaatatgttatcgttataaatatgaaaatattattaaaagaaaagcggaatataatataaatactttatatggaaattttaaaatcattgtgtatatagataaaatcaataatgacattcatttaactttaatatatggctcaatcaatcctaataaaataataccaatatggctaaatcaacaactttcaatatttgattttttacacgccaaaacaagctcatgcacacaaaataatatttcttcaataatgaaaataatgaaattaattaaaaatggtattttaattttagttaattgtgaatattctataaattctatatttaatcaattaaaaaattttaatataattgacgaaaaaaataatattattcaaccaaaagatcataaagatactaatttagttcatattaataatattaatgcgcaaatattaaaagatcttaatatcaaaaaagttaaattaatgacgcatttaaatgatatttcaccttttttaaattttaatataaaaataaaataa

SEQ ID NO: 106 - DNA, 633 bp, Unknown
  Candidatus Liberibacter solanacearum
ttatacttttttttgttgacgccaagcaattaaatctttgattgaaataatttttaatccatgttttattgaaaattcaatgacttgctttccttttttgattgttccatcatcattgactagttcacaaataaccgctacttggggcagtcctgcaattttacataagtcgacagatgcctctgtatgaccaggacggattagcactcctccatcacgagagatgaggggaaagatgtgtcctggacggacaaaatcatttgcagtgctttgtggattagtaagatttttgacagtatgtgtcctatcagcagaagatatacctgtagtaataccatgtttagagtcaacggatacagtaaaagcagttttatggactgattcgttttctaataccataggattaagtccgagtttacgtgcattttgggaaggcatgggtgtgcaaacgattccacaggtatggcgaataataaacgccattttatcggacgtgcaatgtatagctggaagtactaaatcagcttcattttcacgatcatcttcatctgtcacgataaccatgttacctttttgaaaattctggattacatttgaaatatgttgttcattcaatggcattttttgttccat

SEQ ID NO: 107 - DNA, 857 bp, Diaphorina sp.
atggcagaaatgcgacaagagtgtaaatgtcacggcatgtctggatcctgtactgtcaagacctgctggatgaggcttcctaacttcagagttattggagatgctctcaaagatcggtttgacggcgcgtccagagtgatggtcagtaatgaaggtaacgttcgtggcagcagtgtggggaagaaaagtcggtacaacttccagctgaatccgtacaatcctgaacacaaacctccaggtgtgaaggatttggtctacctggaaacctcgccaggattttgtgagaagaatcccgcactgggaatacagggcacgcatggacgacaatgcaatgacaccagtattggtgtagatggttgtgatctgatgtgctgtgggcgaggctacagaacacaggagatcaccgtcgtggaacggtgtgcttgtgcttttatttggtgctgtgaggtcaaatgtaaaacatgtagaacaaagaaaaccattcacacttgcttgtagataatcattattattaatattattagtctaagagaatgattccagaactgtgcctattgatccatacaattacaattgtacgctgtatggtgttgttcagaaagataatctatatagacttgcaaggacctacctacattatagctatgttagtactgaggtaattgccatacaacgatatacatagatcagtacaagtctaaagagtaaatcggccttactgaaatagcagaatatttgctacaggacttggataaattcgtatgaagttatgaacgtaaatgtatctctttcttgcacttttgctacaccttcaacatgaaaatcggacaacattggaaaaattctcgatacttaat

SEQ ID NO: 108 - DNA, 531 bp, Diaphorina citri
atggtcctcaatgtttctctgttacagcatgtgatggcagaaatgcgacaagagtgtaaatgtcacggcatgtctggatcctgtactgtcaagacctgctggatgaggcttcctaacttcagagttattggagatgctctcaaagatcggtttgacggcgcgtccagagtgatggtcagtaatgaaggtaacgttcgtggcagcagtgtggggaagaaaagtcggtacaacttccagctgaatccgtacaatcctgaacacaaacctccaggtgtgaaggatttggtctacctggaaacctcgccaggattttgtgagaagaatcccgcactgggaatacagggcacgcatggacgacaatgcaatgacaccagtattggtgtagatggttgtgatctgatgtgctgtgggcgaggctacagaacacaggagatcaccgtcgtggaacggtgtgcttgtgcttttatttggtgctgtgaggtcaaatgtaaaacatgtagaacaaagaaaaccattcacacttgcttgtag

(19) United States
(12) Patent Application Publication
(10) Pub. No.: US 2025/0027079 A1
(43) Pub. Date: Jan. 23, 2025

(54) USE OF FANA ANTISENSE OLIGONUCLEOTIDES FOR THE PREVENTION AND MANAGEMENT OF HUANGLONGBING AND ZEBRA CHIP DISEASES

(71) Applicants: UNIVERSITY OF FLORIDA RESEARCH FOUNDATION, INCORPORATED, Gainesville, FL (US); THE UNITED STATES OF AMERICA, AS REPRESENTED BY THE SECRETARY OF AGRICULTURE, Washington, DC (US); AUM LIFETECH, INC, Philadelphia, PA (US)

(72) Inventors: Veenu AISHWARYA, Philadelphia, PA (US); Brian Wayne HUNTER, Port St. Lucie, FL (US); Kirsten STELINSKI, Winter Haven, FL (US)

(21) Appl. No.: 18/021,884

(22) PCT Filed: Aug. 18, 2021
(86) PCT No.: PCT/US2021/046568

§ 371 Date: Feb. 17, 2023

Related U.S. Application Data
(60) Provisional application No. 63/067,211, filed on Aug. 18, 2020.

(51) Int. Cl. C12N 15/113; A01N 63/60; A01P 7/04

(52) U.S. Cl. CPC C12N 15/113; A01N 63/60; A01P 7/04; C12N 15/1137; C12Y 599/01003; C12Y 605/01002; C12N 2310/11

(57) ABSTRACT

The present invention is based on the seminal discovery that FANA antisense oligonucleotides inhibit the growth of bacteria harbored within plant-feeding insects, wherein the insect is chewing or feeding from the xylem, phloem, or mesophyll. Specifically, the use of FANA antisense oligonucleotides decreases the expression of target RNA or DNA and inhibits growth of CLas within the psyllid D. citri, which then prevents the transmission of CLas from D. citri to a citrus tree, or of CLso within the psyllid Bactericera cockerelli which prevents the transmission of CLso from Bactericera cockerelli to a Solanaceous plant. Specifically, the use of FANA antisense oligonucleotides decreases the expression of target RNA or DNA and inhibits growth of CLas within the host plant, citrus or Solanaceous, which then prevents the transmission of CLas from D. citri to a citrus tree, or which prevents the transmission of CLso from Bactericera cockerelli to a Solanaceous plant.

### BACKGROUND OF THE INVENTION

### Field of the Invention

The present invention relates to the development of novel strategies for the prevention and management of Huanglongbing disease and specifically relates to the use of FANA antisense oligonucleotides to decrease target RNA expression in plant-infecting microbes and plant-chewing or piercing-sucking plant-feeding insects.

### Background Information

Emerging severe bacterial plant pathogens cause hundreds of millions of dollars of damage to crops annually. One such pathogen, Candidatus Liberibacter asiaticus (CLas) in citrus, is spread in the U.S. by the Asian citrus Psyllid, Diaphorina citri (Hemiptera: Liviidae). CLas bacterial infections result in citrus tree decline, lost yields, and tree death which threatens citrus production worldwide. Related Liberibacter/Psyllid vector pathosystems include the potato psyllid vector, Bactericera cockerelli, and the bacterial pathogen Liberibacter solanacearum, (Lso). (Jessica Vereijssen, Grant R Smith, Phyllis G Weintraub; Bactericera cockerelli (Hemiptera: Triozidae) and Candidatus Liberibacter solanacearum in Potatoes in New Zealand: Biology, Transmission, and Implications for Management, Journal of Integrated Pest Management, Volume 9, Issue 1, 1 Jan. 2018, 13,doi.org/10.1093/jipm/pmy007; Sengoda, V. G., Buchman, J. L. Henne, D. C. Pappu, H. R., Munyaneza, J. E. 2013. “Candidatus Liberibacter solanacearum” titer over time in Bactericera cockerelli (Hemiptera: Triozidae) after acquisition from infected potato and tomato plants. J. Econ. Entomol. 106: 1964-1972.).

Citrus greening or Huanglongbing (HLB) is a devastating disease affecting citrus groves worldwide. HLB symptoms include leaf chlorosis, bitter and undeveloped fruit, premature fruit drop, twig die-back and eventual tree death. The HLB causal agent in Asia, North America and Brazil is Candidatus Liberibacter asiaticus (CLas), a phloem-limited Alphaproteobacterium (Bov6, 2006), which is transmitted by Diaphorina citri Kuwayama (Hemiptera: Liviidae) commonly known as the Asian citrus psyllid (ACP). CLas can be acquire by the psyllid through feeding on the phloem of infected plants. Once inside the digestive tract of the insect, the bacterium can colonize and propagate. Nevertheless, CLas must pass through the gut wall into the hemolymph and reach the salivary glands before it can be successfully transmitted (Hall et al., 2012). Besides CLas, the ACP harbors three known bacterial endosymbionts: Candidatus Carsonella ruddii, a Gammaproteobacterium, which may provide nutritional benefits; Candidatus Profftella armatura, a Betaproteobacterium with a putative defensive role; and wDi, a strain of the Alphaproteobacteria Wolbachia, which are widely distributed amongst insect species. “Morrow, J. J., Hall, A. A. G., Riegler 2017. Symbionts in waiting: the dynamics of incipient endosymbiont complementation and replacement in minimal bacterial communities of psyllids. Microbiome 5:58. DOI 10.1186/s40168-017-0276-4”

Current management of HLB has been focused mainly in controlling ACP populations by applying insecticides. However, the excessive use of insecticides may lead to environmental toxicity, harmful effects on non-target organisms and pest resistance. Alternatives strategies for ACP control, with low environmental impact, may include biological control and RNA interference (RNAi), a form of post-transcriptional gene silencing in which double stranded RNA induces degradation by nucleases of the homologous endogenous transcript.

Zebra chip disease, is disease affecting potato plant. It is caused by an Alphaproteobacteria Candidatus Liberibacter solanacearum (CLso), which is vectored by the potato tomato psyllid Bactericera cockerelli, which infests both potatoes and tomatoes. This bacterium is related to Candidatus Liberibacter spp., which cause citrus greening disease in citrus plants.

Many zebra chip symptoms are evident before the potato is even harvested; foliar signs include chlorosis, leaf scorching, swollen nodes, vascular tissue browning, and curled leaves. Subterranean signs include collapsed stolons, enlarged lenticels, vascular tissue browning, medullary ray discoloration, and necrotic flecking of tuber tissue. However, the main symptom is the development, upon frying of potato tubers from infected plants, of unsightly black lines resembling the stripes of zebras, which renders the chips unsellable. Additionally, striped sections of chips frequently burn and caramelize, resulting in a bitter flavor. “Munyaneza, J. E. 2015. Zebra Chip Disease, Candidatus Liberibacter, and Potato Psyllid: A Global Threat to the Potato Industry. American. J. Potato Research 92:230-235. DOI 10.1007/s12230-015-9448-6”

Plant pathogens, especially bacteria are extremely difficult to target. Few antibiotics are approved for crops and current attitudes towards the risk of antibiotic resistance development have prevented the expansion of antibiotics into crops to reduce bacterial pathogens. As such, the need exists to develop non-antibiotic compositions and methodologies to target such pathogens.

Antisense oligonucleotides (ASOs) are short synthetic oligonucleotides that inhibit or modulate expression of a specific gene by Watson-Crick binding to cellular RNA targets. ASOs act through a number of different mechanisms. Some ASOs bind to an mRNA of a gene of interest, inhibiting expression either by blocking access (steric blocker) of the cellular translation machinery, or by inducing its enzymatic degradation (RNAse-H, RNAse-P). Alternatively, ASOs can target a complementary region of a specific pre-mRNA and modulate its splicing, typically to correct a dysfunctional protein.

FANA (2′-Deoxy-2′-Fluoro-β-D-Arabinonucleic Acid) antisense oligonucleotides are nucleic acids with a phosphorothioate backbone and modified flanking nucleotides, in which the 2′—OH group of the ribose sugar was substituted by a fluorine atom. The flank modifications increase the resistance of the ASOs to degradation and enhance binding to targeted mRNA. The FANA/RNA duplex is recognized by ribonuclease H (RNase H), an enzyme that catalyzes the degradation of duplexed mRNA.

As described herein FANA ASOs may be useful to inhibit the transmission of CLas to a citrus tree by inhibiting the growth of CLas harbored within D. citri and therefore preventing or managing Huanglongbing disease in citrus trees. FANA ASOs may also be useful to inhibit the transmission of CLso to a potato plant by inhibiting the growth of CLso harbored within Bactericera cockerelli and therefore preventing or managing zebra chip disease in potato plant.

As described herein FANA ASOs can be used to target the essential endosymbiotic bacteria needed for psyllid survival, thereby suppressing the vector population leading to reduced transmission of pathogenic bacteria, like CLas and CLso, thus providing an efficient management of Huanglongbing in citrus trees.

### SUMMARY

The present invention is based on the seminal discovery that FANA antisense oligonucleotides inhibit the growth of bacteria harbored within plant-chewing or piercing-sucking plant-feeding insects. Specifically, the use of FANA antisense oligonucleotides decreases the expression of target RNA and inhibits growth of CLas within D. citri which then prevents the transmission of CLas from D. citri to a citrus tree as well as the growth of CLso within Bactericera cockerelli which then prevents the transmission of CLso from Bactericera cockerelli to a potato plant.

In one embodiment the present invention provides a method of decreasing target RNA or DNA expression in a plant-chewing or piercing-sucking plant-feeding insect including providing an antisense oligonucleotide to the insect or to its microbiome, wherein the oligonucleotide includes at least one 2′-deoxy-2′-fluoroarabinonucleotide (2′F-ANA)-modified nucleotide and at least one 2′-deoxyribonucleotide. In many aspects, the target RNA is insect RNA, viral RNA, or bacterial RNA, and the target RNA is DNA Gyrase subunit-A, NAD-dependent Ligase-A, DNA-B Helicase C terminal domain, D-Ribulose A (riboflavin), Ribulose A, B, Acetoacetyl-[acyl-carrier protein], Acetylcholinesterase, Amidophosphoribosyltransferase, Acetyl-CoA carboxylase biotin carboxylase subunit, Biotin Ligase, Biotin-(acetyl-CoA carboxylase)-Ligase, Malonyl Coenzyme A, wsp or 16SrRNA, thereby decreasing target RNA expression. In one aspect, the bacterial RNA is from Candidatus Liberibacter asiaticus (CLas) or from Candidatus Liberibacter solanacearum (CLso). In an additional aspect, the insect is Diaphorina citri (D. citri) or Bactericera cockerelli. In certain aspects, the oligonucleotide has the sequence of one of SEQ ID NOs: 5-77. In a further aspect, the 2′F-ANA modified nucleotides are positioned within the oligonucleotide according to any of Formula 1-16. In some aspects, the insect feeds from plant vascular (xylem or phloem), mesophyll, root, flower, fruit or leave. In certain aspects, the insect ingests the oligonucleotide by consuming plant material containing the oligonucleotide. In an additional aspect, the plant material includes leaf tissue, root tissue, stem tissue, flower tissue, phloem, or a combination thereof. In specific aspects, the plant is a citrus plant or a Solanaceae plant. In a further aspect, the citrus plant is orange, lemon, clementine, lime, grapefruit, pomelo, citron, mandarin, tangelo, trifoliate, rootstock, or ornamental citrus relative. In an additional aspect, the Solanaceous plant is nightshade, potato, tomato, tomatillo, eggplant, bell pepper or capsicum pepper, or related species. In one aspect, the oligonucleotide is: applied to a plant by topical foliar spray, soil applied solution, or irrigation; and/or bound to an absorbent, bound to a peptide, or bound to a synthetic or natural gel, agar, clay, or other material, for application, spreading, mixing with soil, and/or for being applied to soil in solutions or in water, for root uptake or root soaking. In another aspect, the oligonucleotide sequence is a complement to the sequence of the target insect or bacterial RNA, and the oligonucleotide sequence has at least 80%, 85%, 90%, 95%, 98%, 99%, or more sequence identity to the complementary sequence of the target RNA. In many aspects, the target RNA or DNA is prokaryotic or eukaryotic. In various aspects, the target RNA or DNA is selected from the group consisting of messenger RNA (mRNA), microRNA (miRNA), small interfering (siRNA), antisense RNA (aRNA), short hairpin RNA (shRNA), transfer RNA (tRNA), ribosomal RNA (rRNA), small nuclear RNA (snRNA), double-stranded RNA (dsRNA), locked nucleic acid (LNA), Transfer-messenger RNA (tmRNA), viral RNA, viral DNA, polynucleic acids circular ssDNA, and circular DNA. In other aspects, the oligonucleotide sequence has at least 80%, 85%, 90%, 95%, 98%, 99%, or more sequence identity to the complementary RNA or DNA sequence.

In an additional embodiment, the present invention provides a method of inhibiting bacterial growth in a plant-chewing or piercing-sucking plant-feeding insect including providing an antisense oligonucleotide to the insect or to its microbiome, wherein the oligonucleotide includes at least one 2′-deoxy-2′-fluoroarabinonucleotide (2′F-ANA)-modified nucleotide and at least one 2′-deoxyribonucleotide, thereby inhibiting bacterial growth. In one aspect, the oligonucleotide binds to a target RNA or DNA and the target RNA may be DNA Gyrase subunit-A, NAD-dependent DNA Ligase-A, DNA-B helicase C terminal domain, D-Ribulose A (riboflavin), Ribulose A, B, Acetoacetyl-[acyl-carrier protein], Acetylcholinesterase, Amidophosphoribosyltransferase, Acetyl-CoA carboxylase biotin carboxylase subunit, Biotin Ligase, Biotin-(acetyl-CoA carboxylase)-Ligase, Malonyl Coenzyme A, wsp or 16SrRNA. In certain aspects, the RNA is from a gram negative bacteria, a gram positive bacteria, E. coli, Serratia marcescens, an insect endosymbiont, Carsonella rudii, Profftella armigera, Wolbachia-Diaphorina, Wolbachia-Drosophila, Candidatus Liberibacter asiaticus (CLas) or Candidatus Liberibacter solanacearum (CLso). In an additional aspect, the insect is Diaphorina citri (D. citri) or Bactericera cockerelli. In a further aspect, the oligonucleotide has the sequence of one of SEQ ID NOs: 5-77. In one aspect, the insect ingests the oligonucleotide by consuming plant material containing the oligonucleotide. In another aspect, the plant is a citrus plant or a Solanaceae plant. In certain aspects, the citrus plant is of orange, lemon, clementine, lime, grapefruit, pomelo, citron, mandarin, tangelo, trifoliate, rootstock or ornamental citrus relative. In an additional aspect, the Solanaceous plant is nightshade, potato, tomato, tomatillo, eggplant, bell pepper or capsicum pepper. In a further aspect, the oligonucleotide is: applied to a plant by topical foliar spray, soil applied solution, or irrigation; and/or bound to an absorbent, bound to a peptide, or bound to a synthetic or natural gel, agar, clay, cellulose, or other material, for application, spreading, soil amendment and/or for being applied in solution or in water for root uptake or root soaking. In another aspect, the oligonucleotide sequence is a complement to the sequence of the target insect or bacterial RNA or DNA, and the oligonucleotide sequence has at least 80%, 85%, 90%, 95%, 98%, 99%, or more sequence identity to the complementary sequence of the target RNA or DNA.

In a further embodiment, the present invention provides a method of preventing and/or managing of huanglongbing disease in a citrus tree or plant including providing an antisense oligonucleotide to a plant-chewing or piercing-sucking plant-feeding insect or to its microbiome, wherein the oligonucleotide binds to a target RNA or DNA, wherein the oligonucleotide includes at least one 2′-deoxy-2′-fluoroarabinonucleotide (2′F-ANA)-modified nucleotide and at least one 2′-deoxyribonucleotide, and wherein the target RNA is DNA Gyrase subunit-A, NAD-dependent DNA Ligase-A, DNA-B helicase C terminal domain, D-Ribulose A (riboflavin), Ribulose A, B, Acetoacetyl-[acyl-carrier protein], Acetylcholinesterase, Amidophosphoribosyltransferase, Acetyl-CoA carboxylase biotin carboxylase subunit, Biotin Ligase, Biotin-(acetyl-CoA carboxylase)-Ligase, Malonyl Coenzyme A, wsp or 16SrRNA, thereby preventing and/or managing huanglongbing disease. In one aspect, the insect harbors of Candidatus Liberibacter asiaticus (CLas). In another aspect, the oligonucleotide inhibits the growth of CLas. In an additional aspect, inhibiting the growth of CLas prevents the transmission of CLas from the insect to the citrus tree or plant. In certain aspects, the RNA is from Candidatus Liberibacter asiaticus (CLas). In an additional aspect, the insect is an insect: Diaphorina citri (D. citri). In a further aspect, the oligonucleotide has the sequence of one of SEQ ID NOs: 5-77. In one aspect, the insect ingests the oligonucleotide by consuming plant material containing the oligonucleotide. In certain aspects, the citrus plant is of orange, lemon, clementine, lime, grapefruit, pomelo, citron, mandarin, tangelo, trifoliate, rootstock or ornamental citrus relative. In a further aspect, the oligonucleotide is: applied by topical foliar spray, soil applied solution, or irrigation; and/or bound to an absorbent, bound to a peptide, or bound to a synthetic of natural gel, agar, clay, cellulose or other material, for application, spreading, soil amendment and/or for being applied in solution or in water for root uptake or root soaking. In some aspects, the oligonucleotide sequence is a complement to the sequence of the target insect or bacterial RNA or DNA, and the oligonucleotide sequence has at least 80%, 85%, 90%, 95%, 98%, 99%, or more sequence identity to the complementary sequence of the target RNA or DNA.

In a further embodiment, the present invention provides a method of preventing and/or managing of Liberibacter zebra chip disease in a Solanaceous plant including providing an antisense oligonucleotide to a plant-chewing or piercing-sucking plant-feeding insect or to its microbiome, wherein the oligonucleotide includes at least one 2′-deoxy-2′-fluoroarabinonucleotide (2′F-ANA)-modified nucleotide and at least one 2′-deoxyribonucleotide, wherein the oligonucleotide binds to a target RNA or DNA, and wherein the target RNA is DNA Gyrase subunit-A, NAD-dependent DNA Ligase-A, DNA-B helicase C terminal domain, D-Ribulose A (riboflavin), Ribulose A, B, Acetoacetyl-[acyl-carrier protein], Acetylcholinesterase, Amidophosphoribosyltransferase, Acetyl-CoA carboxylase biotin carboxylase subunit, Biotin Ligase, Biotin-(acetyl-CoA carboxylase)-Ligase, Malonyl Coenzyme A, wsp or 16S rRNA, thereby preventing and/or managing the Liberibacter Zebra chip disease. In one aspect, the insect harbors of Candidatus Liberibacter solanacearum (CLso). In another aspect, the oligonucleotide inhibits the growth of CLso. In an additional aspect, inhibiting the growth of CLso prevents the transmission of CLso from the insect to the Solanaceous plant. In certain aspects, the RNA is from Candidatus Liberibacter solanacearum (CLso). In an additional aspect, the insect is an insect: Bactericera cockerelli (B. cockerelli). In a further aspect, the oligonucleotide has the sequence of one of SEQ ID NOs: 5-77. In one aspect, the insect ingests the oligonucleotide by consuming plant material containing the oligonucleotide. In another aspect, the plant is a potato plant, or other suitable plant. In certain aspects, the Solanaceous plant is a: nightshade, potato, tomato, tomatillo, eggplant, bell pepper or capsicum pepper. In a further aspect, the oligonucleotide is: applied by topical foliar sprays, soil applied solutions, or irrigation; and/or bound to an absorbent, bound to a peptide, or bound to a synthetic of natural gel, agar, clay, cellulose or other material, for application, spreading, soil amendment and/or for being applied in solution or in water for root uptake or root soaking. In some aspects, the oligonucleotide sequence is a complement to the sequence of the target insect or bacterial RNA or DNA, and the oligonucleotide sequence has at least 80%, 85%, 90%, 95%, 98%, 99%, or more sequence identity to the complementary sequence of the target RNA or DNA.

In one embodiment, the present invention provides an oligonucleotide including at least one 2′-deoxy-2′-fluoroarabinonucleotide (2′F-ANA)-modified nucleotide and at least one 2′-deoxyribonucleotide and having a sequence of one of SEQ ID NOs: 5-77. In one aspect, the 2′F-ANA-modified nucleotides are positioned according to any of Formulas 1-16. In an additional aspect, the 2′F-ANA-modified nucleotides include internucleotide linkages between nucleotides are phosphodiester bonds, phosphotriester bonds, phosphorothioate bonds (5′O—P(S)O-3O—, 5'S—P(O)O-3′-O—, and 5′O—P(O)O-3'S—), phosphorodithioate bonds, Rp-phosphorothioate bonds, Sp-phosphorothioate bonds, boranophosphate bonds, methylene bonds(methylimino), amide bonds(3′-CH₂—CO—NH-5′ and 3′-CH₂—NH—CO-5′), methylphosphonate bonds, 3′-thioformacetal bonds, (3'S—CH₂—O5′), amide bonds (3′CH₂—C(O)NH-5′), phosphoramidate groups, or a combination thereof. In various aspects, the oligonucleotide is for use in baits, diets, liquids, to target insects, by attraction, or feeding, leading to reduced fitness, fecundity or health of pests, or for the reduction of pathogens, to suppress transmission, or to improve survival of insects considered beneficial to plant health.

### BRIEF DESCRIPTION OF THE DRAWINGS

FIG. 1 shows the localization of FANA oligo in the alimentary canal of D. citri adults. Psyllid guts were dissected and imaged after feeding on Citrus leaves previously treated with a 5 μM solution of an Alexa Fluor 594 labeled oligo. Abbreviations: es, esophagus; fc, filter chamber; am, anterior midgut; pm, posterior midgut; mt, Malpighian tubule.

FIGS. 2A-B show the effect of FANA ASOs on mRNA levels of wDi gyrA in cell culture (A) and CLas LygA in D. citri adults (B). The markers represent the expression of the target gene in the experimental condition compared to its expression in the untreated condition. Values lower than 1 indicates gene suppression. Results are means of triplicate experiments±SE. * P≤0.05; ** P≤0.001.

FIG. 3 depicts the density of viable wDi cells after incubation with 5 μM FANA solutions. Dead wDi refers to bacterial cells that were incubated at 95° C. for 10 minutes. Statistical differences between the treatments (P≤0.05) are designated by different letters (ANOVA: F (3, 8)=614.35; P≤0.0001). Results are means of triplicate experiments±SE.).

FIG. 4 depicts the density of CLas in D. citri samples after feeding 5 μM FANA solutions for seven days. Statistical differences between the treatments (P≤0.05) are designated by different letters (ANOVA: F (4, 40)=10.75; P≤0.0001). Results are means of triplicate experiments±SE.

FIG. 5 shows the effect of FANA ASO on CLas titer in Citrus plants. Treatment effect was expressed as percentage change in CLas titer [(mean titer after treatment−mean titer prior to treatment)/mean titer prior to treatment]×100. Means and standard errors are represented by bars and whiskers, respectively. Bars followed by an asterisk indicates significant change (increase or decrease) in CLas copy number as compared to that before treatment (DPT: days post treatment).

FIG. 6 represents quantification of CLas copy number post treatment with FANA-Ligase A compared to control untreated citrus seedlings.

FIG. 7 shows the effect of FANA ASO on psyllid inoculation efficiency. The figure represents the distribution of CLas titer detected in Citrus macrophylla seedlings (N=15) that were exposed to untreated or FANA-treated infected adult psyllids. Statistical differences between the treatments are designated by * (P≤0.05).

FIG. 8 shows “Growth average” of five potted potato plants per treatment at 52 days post treatments.

FIG. 9 shows “Figure” showing summed Foliar Symptom Severity Scores per 5 potted potato plants, per treatment, covering 52 d post CLso-inoculation period. Scores made at three sampling dates across last 17 days of trial, 29 days post treatment applications.

FIG. 10 shows “Averaged CLso Titers” calculated in bacterial genomes in five potato plants per treatments, at 22 and 32 days post treatment applications. Coincides with average of 12 days post CLso psyllid plant inoculations.

FIGS. 11A-C shows the effects of CLso-DNAB-2 and DNAB4, and CLso-2Gyr-EXT-2 and CLso-2Gyr-EXT4 on potato plants. Specifically, FIG. 11A shows plant height; FIG. 11B shows foliar symptom ranking; and FIG. 11C shows the proportion of symptomatic tubers.

### DETAILED DESCRIPTION

The present invention is based on the seminal discovery that FANA antisense oligonucleotides inhibit the growth of bacteria harbored within plant-chewing or piercing-sucking plant-feeding insects. Specifically, the use of FANA antisense oligonucleotides decreases the expression of target RNA and inhibits growth of CLas within D. citri, which then prevents the transmission of CLas from D. citri to a citrus tree.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

“Citrus” as used herein refers to any species of tree producing any variety of citrus fruit, such as oranges, tangerines, clementines, lemons, limes, and the like, or wildtype citrus relatives which may also be used as ornamental plants.

“Solanaceae’ or “Solanaceous plant” refers to the family of flowering plants (Order: Solanales), which include 102 genera and nearly 2,500 species. Examples of Solanaceous plant include, but are not limited to, potato, tomato, eggplant, pepper and nightshade.

For the purpose of the invention, the “complement of a nucleotide sequence X” is the nucleotide sequence which would be capable of forming a double-stranded DNA or RNA molecule with the represented nucleotide sequence, and which can be derived from the represented nucleotide sequence by replacing the nucleotides by their complementary nucleotide according to Chargaff's rules (A< >T; G< >C; A< >U) and reading in the 5′ to 3′ direction, i.e., in opposite direction of the represented nucleotide sequence. In the context of the present disclosure, this term also includes synthetic analogs of DNA/RNA (e.g., 2′F-ANA oligos).

The term “effective amount” of a composition provided herein refers to the amount of the composition capable of performing the specified function for which an effective amount is expressed. The exact amount required can vary from composition to composition and from function to function, depending on recognized variables such as the compositions and processes involved. An effective amount can be delivered in one or more applications. Thus, it is not possible to specify an exact amount, however, an appropriate “effective amount” can be determined by the skilled artisan via routine experimentation.

“Insect” or “insect pest” as used herein means any variety of insects that may cause harm to plants, trees, fruits, or nuts or products produced thereby or therefrom. In exemplary embodiments, such pests include leaf-eating and sap-feeding arthropods, such as the Asian citrus psyllid.

“Microbiome” or “insect microbiome” as used herein refers to the community of microorganisms—such as bacteria, archaea, fungi, as well as viruses—that inhabit the insect ecosystem. In the soil for example, microbes are essential for supporting plant life by mediating uptake and entry of nutrients into the food chain, cycling carbon and nitrogen, breaking down pollutants and much more. By affecting an insect microbiome, one can also affect said insect; as such the oligonucleotides of the present invention may be provided to the insect directly, or indirectly through delivery of the oligonucleotide to its microbiome.

The term “plant” includes whole plants, plant organs, progeny of whole plants or plant organs, embryos, somatic embryos, embryo-like structures, protocorms, protocorm-like bodies (PLBs), and suspensions of plant cells. Plant organs include, e.g., shoot vegetative organs/structures (e.g., leaves, stems and tubers), roots, flowers and floral organs/structures (e.g., bracts, sepals, petals, stamens, carpels, anthers and ovules), seeds (including embryo, endosperm, and seed coat) and fruit (the mature ovary), plant tissue (e.g., vascular tissue, ground tissue, and the like) and cells (e.g., guard cells, egg cells, trichomes and the like).

As used herein, “preventing” a disease refers to inhibiting the full development of a disease.

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, it will be understood that modifications and variations are encompassed within the spirit and scope of the instant disclosure. The preferred methods and materials are now described.

In one embodiment the present invention provides a method of decreasing target RNA or DNA expression in a plant-chewing or piercing-sucking plant-feeding insect including providing an antisense oligonucleotide to the insect or to its microbiome, wherein the oligonucleotide includes at least one 2′-deoxy-2′-fluoroarabinonucleotide (2′F-ANA)-modified nucleotide and at least one 2′-deoxyribonucleotide, thereby decreasing target RNA or DNA expression. In one aspect the target RNA is insect RNA, viral RNA, or bacterial RNA. In some aspects, the target RNA is bacterial RNA from Candidatus Liberibacter asiaticus (CLas). In certain aspects the target RNA is Gyrase subunit-A, NAD-dependent DNA Ligase-A (Ligase A); DnaB-like helicase C terminal domain (DnaB), D-Ribulose A (riboflavin), Ribulose A, B, Acetoacetyl-[acyl-carrier protein], Acetylcholinesterase, Amidophosphoribosyltransferase, Acetyl-CoA carboxylase biotin carboxylase subunit, Biotin Ligase, Biotin-(acetyl-CoA carboxylase)-Ligase, Malonyl Coenzyme A, wsp or 16S rRNA. In an additional aspect, the insect is Diaphorina citri (D. citri) or Bactericera cockerelli. In certain aspects, the oligonucleotide has the sequence of SEQ ID NOs: 5-77. In a further aspect, the 2′F-ANA modified nucleotides are positioned within the oligonucleotide according to any of Formula 1-16. In some aspects, the insect feeds from plant vascular (xylem or phloem), mesophyll, root, flower, fruit or leave. In certain aspects, the insect ingests the oligonucleotide by consuming plant material containing the oligonucleotide. In an additional aspect, the plant material includes leaf tissue, root tissue, stem tissue, flower tissue, phloem, or a combination thereof. In specific aspects, the plant is a citrus plant or a Solanaceae plant. In a further aspect, the may be any citrus plant or relative such as orange, lemon, clementine, lime, grapefruit, pomelo, citron, mandarin, tangelo, or any trifoliate, or rootstock, or ornamental citrus relative. In an additional aspect, the Solanaceae plant is nightshade, potato, tomato, tomatillo, eggplant, bell pepper or capsicum pepper, family of flowering plants (Order: Solanales). In one aspect, the oligonucleotide is: applied to a plant by topical foliar spray, soil applied solution, or irrigation; and/or bound to an absorbent, bound to a peptide, or bound to a synthetic or natural gel, agar, clay, cellulose or other material, for application, spreading, soil amendment and/or for being applied in solution or in water for root uptake or root soaking.

Antisense oligonucleotides of the present invention are single-stranded deoxyribonucleotides complementary to a targeted mRNA or DNA. Hybridization of an ASO to its target mRNA via Watson-Crick base pairing can result in specific inhibition of gene expression by various mechanisms, depending on the chemical make-up of the ASO and location of hybridization, resulting in reduced levels of translation of the target transcript (Crooke 2004). ASOs of the present invention typically encompass oligonucleotides having at least one sugar-modified nucleoside (e.g., 2′F-ANA) as well as naturally-occurring 2′-deoxy-nucleosides (see, e.g., U.S. Pat. No. 8,278,103 which is specifically incorporated by reference). ASO-induced protein knockdown is usually achieved by induction of RNase H endonuclease activity. When activated, the RNAse H cleaves the RNA-DNA heteroduplex leading to the degradation of the target mRNA. This leaves the ASO intact so that it can function again (Wu et al, J. Biol. Chem. (2004) 279:181-9).

As provided herein, ASOs can be utilized by environmental application (e.g., root soak, foliar spray) to plants in horticultural and agricultural settings. ASOs solve the problems encountered with topically applied nucleotide-based strategies (e.g., dsRNA) which are subject to environmental degradation. The ASO technology provides greater stability due to modifications of the backbone of the single-stranded RNA/DNA hybrid molecule, and increases resistance to degradation by using synthetic nucleotides in the structure.

While there are many types of ASO's, the main discoveries in ASO development included two main chemical modifications. These modifications include the 2′-fluoro (2′-F) substitutions and the phosphorothioate chemistry. These two modifications constitute synthetic analogs of naturally occurring nucleic acids, but which have greater stability and activity. Thus, some embodiments of the present invention use 2′-F substitutions, and modification of the sugar backbone with phosphorothioate chemistry to produce ASOs containing 2′-deoxy-2′-fluoro-β-D-arabinonucleic acid (2′F-ANA), termed “FANA antisense oligonucleotides” (FANA-ASO). The stability, specificity and non-toxicity of FANA-ASO oligonucleotides make them good candidates for difficult to control pathogens involving microbes and insects.

As detailed further herein, FANA-ASO can be applied to plants as topical spray applications to foliage, soils, or in baits or inert carriers, and results in suppression of RNA targets in bacterial pathogens of plants, and insects, and the endosymbionts in arthropods that feed on treated plants or baits. These results demonstrate that for chewing insect pests, topical applications of ASO are effective in targeting Lepidoptera and Coleoptera pests. As for plant-feeding hemipterans, like sap-sucking insects—psyllids, aphids, leafhoppers, whitefly, planthoppers and other Hemiptera—the FANA-ASOs are delivered into, and move systemically through the vascular tissues of plants allowing for these insects to ingest the FANA-ASO when they feed. Our results demonstrate that, similarly to other small nucleic acids that are topically applied to foliage as a spray, or the soil root zone of plants, the FANA-ASOs of the present invention are absorbed through leaves and roots and then move systemically throughout the plant within hours.

As described herein, plant delivery strategies, using exogenously-applied FANA-ASO, is applicable for diverse plant species (citrus, grapevines, woody ornamentals, sunflowers, basil, okra) and insects from several arthropod Orders: Hemiptera, Coleoptera, Lepidoptera, Diptera. Together, this data indicates that the novel approaches described herein can be utilized to target many Arthropods and that a wide variety of plants are broadly amenable to the novel treatment methodologies with ASO products described herein. As used herein “modified antisense oligonucleotide” refers to synthetic antisense oligonucleotides (AONs) containing modified sugar. AONs are single stranded oligonucleotides that recognize nucleic acid sequences via Watson-Crick base pairing and cause pre- or post-transcriptional gene silencing. The AON binds to its target mRNA, and forms a duplex that is recognized by RNase H, which in turn induces the cleavage of the mRNA, the steric blocking of translation machinery, or the prevention of necessary RNA interactions, or modification of natural interactions.

### FANA Antisense Oligonucleotides

The chemistry and construction of 2′F-ANA oligonucleotides (also termed FANA or FANA-ASO) has been described elsewhere in detail (See, e.g., U.S. Pat. Nos. 8,278,103 and 9,902,953). The FANA-ASOs and methods of using them disclosed herein contemplate any FANA chemistries known in the art. In some embodiments, a FANA-ASO includes an internucleoside linkage including a phosphate, thereby being an oligonucleotide. In some embodiments, the sugar-modified nucleosides and/or 2′-deoxynucleosides include a phosphate, thereby being sugar-modified nucleotides and/or 2′-deoxynucleotides. In some embodiments, a FANA-ASO includes an internucleoside linkage including a phosphorothioate. In some embodiments, the internucleoside linkage is selected from phosphorothioate, phosphorodithioate, methylphosphorothioate, Rp-phosphorothioate, Sp-phosphorothioate. In some embodiments, the a FANA-ASO includes one or more internucleotide linkages selected from: (a) phosphodiester; (b) phosphotriester; (c) phosphorothioate; (d) phosphorodithioate; (e) Rp-phosphorothioate; (f) Sp-phosphorothioate; (g) boranophosphate; (h) methylene (methylimino) (3′CH₂—N(CH₃)—O5′); (i) 3′-thioformacetal (3'S CH₂—O5′); (j) amide (3′CH₂ —C(O)NH-5′); (k) methylphosphonate; (1) phosphoramidate (3′-OP(O₂)—N5′); and (m) any combination of (a) to (1).

In some embodiments, FANA-ASOs including alternating segments or units of sugar-modified nucleotides (e.g., arabinonucleotide analogues [e.g., 2′F-ANA]) and 2′-deoxyribonucleotides (DNA) are utilized. In some embodiments, a FANA-ASO disclosed herein includes at least 2 of each of sugar-modified nucleotide and 2′-deoxynucleotide segments, thereby having at least 4 alternating segments overall. Each alternating segment or unit may independently contain 1 or a plurality of nucleotides. In some embodiments, each alternating segment or unit may independently contain 1 or 2 nucleotides. In some embodiments, the segments each include 1 nucleotide. In some embodiments, the segments each include 2 nucleotides. In some embodiments, the plurality of nucleotides may consist of 2, 3, 4, 5 or 6 nucleotides. A FANA-ASO may contain an odd or even number of alternating segments or units and may commence and/or terminate with a segment containing sugar-modified nucleotide residues or DNA residues. Thus, a FANA-ASO may be represented as follows:

A₁-D₁-A₂-D₂-A₃-D₃ . . . A_{z}-D_{z}

Where each of A₁, A₂, etc. represents a unit of one or more (e.g., 1 or 2) sugar-modified nucleotide residues (e.g., 2′F-ANA) and each of D₁, D₂, etc. represents a unit of one or more (e.g., 1 or 2) DNA residues. The number of residues within each unit may be the same or variable from one unit to another. The oligonucleotide may have an odd or an even number of units. The oligonucleotide may start (i.e. at its 5′ end) with either a sugar-modified nucleotide-containing unit (e.g., a 2′F-ANA-containing unit) or a DNA-containing unit. The oligonucleotide may terminate (i.e. at its 3′ end) with either a sugar-modified nucleotide-containing unit or a DNA-containing unit. The total number of units may be as few as 4 (i.e. at least 2 of each type).

In some embodiments, a FANA-ASO disclosed herein includes alternating segments or units of arabinonucleotides and 2′-deoxynucleotides, wherein the segments or units each independently include at least one arabinonucleotide or 2′-deoxynucleotide, respectively. In some embodiments, the segments each independently include 1 to 2 arabinonucleotides or 2′-deoxynucleotides. In some embodiments, the segments each independently include 2 to 5 or 3 to 4 arabinonucleotides or 2′-deoxynucleotides. In some embodiments, a FANA-ASO disclosed herein includes alternating segments or units of arabinonucleotides and 2′-deoxynucleotides, wherein the segments or units each include one arabinonucleotide or 2′-deoxynucleotide, respectively. In some embodiments, the segments each independently include about 3 arabinonucleotides or 2′-deoxynucleotides. In some embodiments, a FANA-ASO disclosed herein includes alternating segments or units of arabinonucleotides and 2′-deoxynucleotides, wherein the segments or units each include one arabinonucleotide or 2′-deoxynucleotide, respectively. In some embodiments, a FANA-ASO disclosed herein includes alternating segments or units of arabinonucleotides and 2′-deoxynucleotides, wherein said segments or units each include two arabinonucleotides or 2′-deoxynucleotides, respectively.

In some embodiments, a FANA-ASO disclosed herein has a structure selected from:

a)(Aₓ-D_{y})ₙ I

b)(D_{y}-Aₓ)ₙ II

c)(Aₓ-D_{y})ₘ-Aₓ-D_{y}-Aₓ III

d) (D_{y}-Aₓ)ₘ-D_{y}-Aₓ-D_{y} IV

wherein each of m, x and y are each independently an integer greater than or equal to 1, n is an integer greater than or equal to 2, A is a sugar-modified nucleotide and D is a 2′-deoxyribonucleotide. For example, a FANA-ASO disclosed herein has structure I wherein x=1, y=1 and n=10, thereby having a structure:

A-D-A-D-A-D-A-D-A-D-A-D-A-D-A-D-A-D-A-D.

In another example, a FANA-ASO disclosed herein has structure II wherein x=1, y=1 and n=10, thereby having a structure:

D-A-D-A-D-A-D-A-D-A-D-A-D-A-D-A-D-A-D-A.

In another example, a FANA-ASO disclosed herein has structure III wherein x=1, y=1 and n=9, thereby having a structure:

A-D-A-D-A-D-A-D-A-D-A-D-A-D-A-D-A-D-A-D-A.

In another example, a FANA-ASO disclosed herein has structure IV wherein x=1, y=1 and n=9, thereby having a structure:

D-A-D-A-D-A-D-A-D-A-D-A-D-A-D-A-D-A-D-A-D.

In another example, a FANA-ASO disclosed herein has structure I wherein x=2, y=2 and n=5, thereby having a structure:

A-A-D-D-A-A-D-D-A-A-D-D-A-A-D-D-A-A-D-D.

In another example, a FANA-ASO disclosed herein has structure II wherein x=2, y=2 and n=5, thereby having a structure:

D-D-A-A-D-D-A-A-D-D-A-A-D-D-A-A-D-D-A-A.

In another example, a FANA-ASO disclosed herein has structure III wherein x=2, y=2 and m=4, thereby having a structure:

A-A-D-D-A-A-D-D-A-A-D-D-A-A-D-D-A-A-D-D-A-A.

In another example, a FANA-ASO disclosed herein has structure IV wherein x=2, y=2 and m=4, thereby having a structure:

D-D-A-A-D-D-A-A-D-D-A-A-D-D-A-A-D-D-A-A-D-D.

Specific examples of modified synthetic FANA-ASOs described herein include the FANA-ASOs shown in Tables 1 and 5 below:

TABLE 1
FANAs sequences

| FANA | | |
|---|---|---|
| Descriptor | Sequence | SEQ ID NO: |
| SC-ASO-1 | ACTGGGATACGACAAGGATAT | SEQ ID NO:1 |
| SC-ASO-2 | TCTTGGAACAGCATAGGGACA | SEQ ID NO:2 |
| SC-ASO-3 | ATATCCTTGTCGTATCCCAGT | SEQ ID NO:3 |
| SC-ASO-4 | AUAUCCTTGTCGTATCCCAGU | SEQ ID NO: 4 |
| | >X57174.1 E. coli | |
| E. coli bacteria | gyrA gene for DNA | |
| DNA gyrase subunit A | gyrase subunit A | |
| ECol_GyrA_1 | ATGAGCGACCTTGCGAGAGAA | SEQ ID NO: 5 |
| ECol_GyrA_2 | ATTCTCGCTGCGTTATATGCT | SEQ ID NO: 6 |
| ECol_GyrA_3 | GCGGCAATGCGTTATACGGAA | SEQ ID NO: 7 |
| Ecol_GyrA_4 | CGACTGCTGGTGGCGAACACT | SEQ ID NO: 8 |
| | >NZ_HG326223.1: | |
| | c2820748-2818106 | |
| | Serratia marcescens | |
| Serratia marcescens | subsp. marcescens | |
| DNA gyrase subunit A | Db11 | |
| Smarc_GyrA_1 | ATGAGCGACCTTGCCAGAGAA | SEQ ID NO: 9 |
| Smarc_GyrA_2 | GTTTTCACTGCGCTACATGCT | SEQ ID NO: 10 |
| Smarc_GyrA_3 | GCGGCGATGCGTTATACCGAA | SEQ ID NO: 11 |
| Smarc_GyrA_4 | CGCCTGCTGGTGGCCAACACC | SEQ ID NO: 12 |
| Liberibacter asiaticus | DNA gyrase subunit A | |
| bacteria | Gyrase A. | |
| CLas-GYRA-1 | TGAAGGACAAGGAAATTTTGG | SEQ ID NO: 13 |
| CLas, GyrA-2 | GAAGGACAGGGAAAATTTTTG | SEQ ID NO: 14 |
| | CLso-1 Gyrase-A | |
| L. solanacearum | infected Potato | |
| L. solanacearum | GATTGAAGGGCAGGGGAATTC | SEQ ID NO: 15 |
| CLso-1 Gyrase-A | | |
| host Potato | | |
| L. solanacearum CLso2 | GAAGAACGGGCGAACTTTTTG | SEQ ID NO: 16 |
| -GyrA host Potato | | |
| CLso-1 GyrA-Ext-1 | AATAAACGCAATGACCAATCC | SEQ ID NO: 17 |
| host Potato | | |
| CLso-2 Gyr-Ext-2 | ATAACCTTGACGATCTGATTC | SEQ ID NO: 18 |
| CLso-2 Gyr-Ext-4 | TTCTATCAGGGTAAATCGCTC | SEQ ID NO: 19 |
| L. asiaticus, | D-ribulose | |
| in Citrus Ribulose | CLas Ribulose B gene | |
| Liberibacter asiaticus- | GTCGTTAGATGAACGATATAT | SEQ ID NO: 20 |
| CLIBRIB-1 | | |
| Liberibacter asiaticus- | CCAAGATCTAATTGCTTGGCG | SEQ ID NO: 21 |
| CLIBRIB-2 | | |
| Liberibacter asiaticus- | GCTGACTTAGTACTTGCCGCC | SEQ ID NO: 22 |
| CLIBRIB-3 | | |
| Liberibacter asiaticus- | CTCCGATAGCGGTTATTTGCG | SEQ ID NO: 23 |
| CLIBRIB-4 | | |
| Wolbachia-Diaphorina | Psyllid Endosymbiont | |
| endosymbiont | | |
| AUM-C1-Wdi-aCoA-2 | ACUUUCCTAAAACAAGAGGCC | SEQ ID NO: 24 |
| AUM-C1-Wdi-aCoA-3 | GUUCCAUAGGTAATCCUGAAU | SEQ ID NO: 25 |
| AUM-C1-Wdi-aCoA-4 | AAGAGUGCAGCTTAUCGUUGU | SEQ ID NO: 26 |
| -Wolbachia-Diaphorina- | Psyllid Endosymbiont | |
| Amidophosphoribosyl- | Amidophosphoribosyl- | |
| transferase | transferase | |
| AUM-C1-WDil | GCGTAGTAACCAGTAACAACG | SEQ ID NO: 27 |
| AUM-C1-WDi2 | GGCCTCTTGTTTTAGGAAAGT | SEQ ID NO: 28 |
| AUM-C1-WDi3 | ATTCAGGATTACCTATGGAAC | SEQ ID NO: 29 |
| AUM-C1-WDi4 | ACAACGATAAGCTGCACTCTT | SEQ ID NO: 30 |
| AUM-C1-WDi5 | GTTCTTACGTTCTTGCATCTGA | SEQ ID NO: 31 |
| | Acteylcholinesterase | |
| Wolbachia-Diaphorina | below | |
| AUM-C1-WDi-aCoA-1 | CGUUGUUACTGGTTACUACGC | SEQ ID NO: 32 |
| AUM-C1-WDi-aCoA-2 | ACUUUCCTAAAACAAGAGGCC | SEQ ID NO: 33 |
| AUM-C1-WDi-aCoA-3 | GUUCCAUAGGTAATCCUGAAU | SEQ ID NO: 34 |
| AUM-C1-WDi-aCoA-4 | AAGAGUGCAGCTTAUCGUUGU | SEQ ID NO: 35 |
| AUM-C1-WDi-aCoA-5 | UCAGAUGCAAGAACGUAAGAAC | SEQ ID NO: 36 |
| | TARGET SEQUENCE (TS) 5′-3′ | |
| | Wolb-Dc endosymbiont | |
| Wolbachia-Diaphorina | DNA gyrase subunit A | |
| AUM_WLBDC-GyrA 1 | TGATGGGCAAGGTAACTTTGG | SEQ ID NO: 37 |
| AUM_WLBDC-GyrA 2 | TGGTATGGCAACCAATATTCC | SEQ ID NO: 38 |
| AUM_WLBDC-GyrA 3 | GGTGTTGCAGTTGGTATGGCA | SEQ ID NO: 39 |
| AUM_WLBDC-GyrA 4 | CCAGGGCCGGATTTTCCAACG | SEQ ID NO: 40 |
| Wolb-Dc- Gyrase A-1. | CATTCAAGAAGGACAAGGAAA | SEQ ID NO: 41 |
| | Wolbachia endosymbiont | FLY |
| Wolbachia-Drosophila | of Drosophila | |
| simulans (Fruit Fly) | simulans WLB-Ds>WP_015589386.1 | |
| Amido- | amidophosphoribosyl-transferase | |
| Phosphoribosyltransferase | [Wolbachia endosymbiont | |
| Wolb-Ds + WLBDS | of Drosophila simulans] | |
| AUM-WLBDS-1 | CGATAAGCTGCACTCTTATCA | SEQ ID NO: 42 |
| AUM-WLBDS-2 | GGTGCTTATTCTTTTGTTGCA | SEQ ID NO: 43 |
| AUM-WLBDS-3 | TAGCCTCAATGTTTCCTTTCC | SEQ ID NO: 44 |
| AUM-WLBDS-4 | GGATATGCAAAGCATTCAGGA | SEQ ID NO: 45 |
| Carsonella ruddii | D. citri, Psyllid Endosymbiont | |
| Psyllid endosymbiont | Gyrase-A | |
| AUM-DCRUDD1 | CAATTAATGGGAAAATTTCCC | SEQ ID NO: 46 |
| AUM-DCRUDD2 | GTTTGTAGGAACATAAATTTCC | SEQ ID NO: 47 |
| AUM-DCRUDD3 | CAACAATTGTATCATATAAAGG | SEQ ID NO: 48 |
| AUM-DCRUDD4 | CTTTAATAAAATAATATTCGA | SEQ ID NO: 49 |
| Profftella armatura | | |
| Psyllid endosymbiont- | Diaphorina citri Psyllid | |
| Gyrase-A | Endosymbiont Gyrase-A | |
| Profftella armatura-CPFF1 | GCTGCGGCAATGCGTTATACT | SEQ ID NO: 50 |
| Profftella armatura- | CCAACAGCAGGTATTATTTATG | SEQ ID NO: 51 |
| CPFF2 | | |
| Profftella armatura- | CCTTTTGTAAAATGTGCACGA | SEQ ID NO: 52 |
| CPFF3 | | |
| Profftella armatura- | CATGAAATGAATAATTTATGG | SEQ ID NO: 53 |
| CPFF4 | | |
| Profftella armatura, | ATTAGTAGATGGACAAGGTAA | SEQ ID NO: 54 |
| GyrA-5 | | |
| Profftella armatura | D. citri, Psyllid Endosymbiont | |
| Psyllid endosymbiont | D-ribulose (riboflavin synthesis) | |
| Profftella armatura- | CCAACCTGGACACATTTTTCC | SEQ ID NO: 55 |
| CPFFRIB-1 | | |
| Profftella armatura- | GAGGAGTATTAATACGAGGAG | SEQ ID NO: 56 |
| CPFFRIB-2 | | |
| Profftella armatura- | GAGGAGGTCATACTGAAGCTG | SEQ ID NO: 57 |
| CPFFRIB-3 | | |
| Profftella armatura- | GAAGGAGATATTATATTATCT | SEQ ID NO: 58 |
| CPFFRIB-4 | | |
| Profftella armatura- | GAAGGAGATATTATATTATCT | SEQ ID NO: 59 |
| CPFFRIB-5 | | |
| Liberibacter asiaticus | CLas, DnaB-Helicase C | |
| DnaB-Helicase C | in Citrus | |
| CLASDNAB_1 | CTATCCTTGTGAATAATGAGGC | SEQ ID NO: 60 |
| CLASDNAB_2 | GCTTCCGAAGCAGTTTCAATAA | SEQ ID NO: 61 |
| CLASDNAB_3 | GGCTGGACAAGCGTTTAATCG | SEQ ID NO: 62 |
| CLASDNAB_4 | TATTCTTGCTTATACAAAGTG | SEQ ID NO: 63 |
| Liberibacter | | |
| solanacearum | | |
| CLso- | CLso DnaB-Helicase C | |
| DnaB-Helicase C | in Potato | |
| CLsoDNAB-1 | GCATCATTATTCACTAGGATAG | SEQ ID NO: 64 |
| CLsoDNAB-2 | TATGGAAACAGCCTCAGATGC | SEQ ID NO: 65 |
| CLsoDNAB-3 | CGATTAAAAGCTTGTCCAGCC | SEQ ID NO: 66 |
| CLsoDNAB-4 | CATTTTGTATATGCGAGGACA | SEQ ID NO: 67 |
| CLsoDnaB-5 | TTGTCCAGCCATATCAATAGC | SEQ ID NO: 68 |
| CLsoDnaB-6 | TAGTTCCGATTTGTATGCATC | SEQ ID NO: 69 |
| CLso-DnaB-7 | TGTATATGCGAGGACATCGTC | SEQ ID NO: 70 |
| Liberibacter asiaticus, | | |
| CLas | | |
| NAD-dependent DNA | | |
| Ligase-A | NAD-dependent DNA Ligase-A | |
| CLas-LigA-1 | CGGCACAGCAATTAGAACGTC | SEQ ID NO: 71 |
| CLas-LigA-2 | GGAAGGGATATTCGTGTAGG | SEQ ID NO: 72 |
| CLas-LigA-3 | CGGAGAGGATGTTAGTGCTTGT | SEQ ID NO: 73 |
| CLas-Liga-4 | CCTGATTTAGCACGCTCTGAG | SEQ ID NO: 74 |
| Liberibacter | | |
| solanacearum | | |
| CLSO, | CLso, Gyrase A | |
| CLso-2Gyr-Ext-3 | TATTACGATAGATCCTCGACC | SEQ ID NO: 75 |
| CLso-2Gyr | CAAATCAGGTATAGCACGGCC | SEQ ID NO: 76 |
| CLso-4Gyr | GGGAATATTCGTCGCCATACC | SEQ ID NO: 77 |

The formulas shown in Table 2 may be applied to any of SEQ ID NOs: 1-77, or a portion thereof, wherein X represents a nucleotide (A, C, G, T, or U), and wherein bold and underlined nucleotides represent sugar-modified or 2′F-ANA-modified nucleotide with backbone phosphorothioate linkages.

TABLE 2
Exemplary 2′F-ANA nucleoside
placement within 21-mer FANA-ASOs

| 21 nucleotides | Formula |
|---|---|
| Formula 1 | XXXXXXXXXXXXXXXXXXXXX |
| Formula 2 | XXXXXXXXXXXXXXXXXXXXX |
| Formula 3 | XXXXXXXXXXXXXXXXXXXXX |
| Formula 4 | XXXXXXXXXXXXXXXXXXXXX |
| Formula 5 | XXXXXXXXXXXXXXXXXXXXX |
| Formula 6 | XXXXXXXXXXXXXXXXXXXXX |
| Formula 7 | XXXXXXXXXXXXXXXXXXXXX |
| Formula 8 | XXXXXXXXXXXXXXXXXXXXX |
| Formula 9 | XXXXXXXXXXXXXXXXXXXXX |
| Formula 10 | XXXXXXXXXXXXXXXXXXXXX |
| Formula 11 | XXXXXXXXXXXXXXXXXXXXX |
| Formula 12 | XXXXXXXXXXXXXXXXXXXXX |
| Formula 13 | XXXXXXXXXXXXXXXXXXXXX |
| Formula 14 | XXXXXXXXXXXXXXXXXXXXX |
| Formula 15 | XXXXXXXXXXXXXXXXXXXXX |
| Formula 16 | XXXXXXXXXXXXXXXXXXXXX |

### FANA-ASO Compositions

In particular embodiments, the present invention provides a composition having an inhibitory FANA-ASO represented by one or more of SEQ ID NOs. 1-77, and having 2′F-ANA placement within the FANA-AS O as shown in any of Formulas 1-16 (Table 3) and specific for a target mRNA or fragment thereof. Other placements of 2′F-ANA oligonucleo sides within a FANA-ASO are also contemplated, as are FANA-ASOs of 10, 11, 12, 13, 14, 15, 16, 17, 18, 19, 20, 21, 22, 23, 24, 25, 26, 27, 28, 29, 30, or more bases in length. Typically, FANA-ASOs of the present invention are provided to a target recipient (e.g., plant, insect or bacteria) in an amount sufficient to induce RNA silencing, thereby inhibiting production of the polypeptide encoded by one or more of the full-length genes targeted by SEQ ID NOs: 5-77. For example, a FANA-ASO of the present invention is applied to a plant topically, allowing for uptake of the FANA-ASO by the plant. The FANA-ASO can control a bacterial pathogen currently infecting the plant. Additionally, when a plant pest (e.g., D. citri) is feeding on a treated plant, the insect can ingest a sufficient level of the FANA-ASO to control or kill bacteria harbored by the insect pest and/or control or kill the insect pest itself.

In addition to a FANA-ASO of the present invention, compositions of the present invention intended to be applied to a plant can be formulated so as to contain one or more phagostimulants, pesticides, fungicides, or combinations thereof. The composition can be formulated to be coated on a plant, plant part, plant tissue (e.g., root or leaf), or seed. In certain aspects the FANA-ASO is combined with one or more excipients, buffering agents, carriers, etc. Such components are well known in the art and readily chosen for various applications by one skilled in the art.

Typically, a FANA-ASO of the present invention is provided to a target insect pest, target plant in need of treatment, or target microbe in an amount sufficient to inhibit production of the polypeptide encoded by one or more of the full-length genes targeted by FANA-ASOs. For example, when an insect pest is feeding on FANA-ASO-laden plant material (e.g., leaf), the insect ingests a sufficient level of FANA-ASO to result in a phenotypic effect on a bacterium harbored in its gut. In some embodiments, a combination of two or more FANA-ASOs can be combined in a single plant. In embodiments where two or more FANA-ASOs are combined in a single plant, the FANA-ASOs can target different genes or different portions of the same gene from the same or different targets. Thus, in one embodiment, a single plant material can be used to deliver multiple, different FANA-ASOs targeting the production of one or more proteins made by the treated plant, the insect pest, and/or a microbe present in the plant or in the insect. Where two or more FANA-ASOs are taken up and distributed throughout the plant material, the FANA-ASOs can be provided to the plant in a single solution, or in multiple, sequentially-applied solutions.

In addition to FANA-ASOs, compositions of the present invention that are intended to be applied to a plant can also include one or more chemoattractants, phagostimulants, visual attractants, insecticides, pheromones, fungicides, or combinations thereof. Such additional components are well known in the art and are readily chosen to complement compositions of the present invention, but are not specifically integral to the present invention. These additional components can be formulated to be coated on a plant, plant part, leaf, fruit, vegetable, stem or other plant structure. In certain aspects the additional component(s) are combined with one or more excipients, buffering agents, carriers, etc. that are also well known in the art.

Where additional components are applied in a coating, the coating can be formulated as a spray or dip so that the additional non-FANA-ASO components remain on the exterior of the plant material. For example, a leaf having a FANA-ASO distributed through at least part of its vascular system can be coated with a composition including one or more chemoattractants, phagostimulants, visual attractants, insecticides, pheromones, fungicides, or combinations thereof. Alternately, the additional component can be mixed with an aqueous solution containing the FANA-ASO(s) to be taken up and distributed via vascular action of the plant material, or osmosis through the plant material, thus distributing the FANA-ASO(s) and the additional component(s) throughout at least part of the plant material.

### Application to Target Plants

Compositions of the invention disclosed herein can be applied to soil, fruits, vegetables, crops, and any other desired target using any delivery methodology known to those of skill in the art. For example, FANA-ASO-containing compositions can be applied to the desired locale via methods and forms including, but not limited to, shank injection, sprays, granules, flood/furrow methods, sprinklers, fumigation, root soaking and drip irrigation. In embodiments of the invention where the compositions are sprayed onto a desired locale, the compositions can be delivered as a liquid suspension, emulsion, microemulsion or powder. In other embodiments, granules or microcapsules can be used to deliver the compositions of the invention. The compositions of the invention might be applied with traditional agricultural methods, as topical foliar sprays, soil applied solutions, irrigation, or as bound to absorbent, or clay for spreading applications, and root soaking as in hydroponic systems.

The compositions of the present invention can be applied to plants and/or crops by any convenient method, for example, by using a fixed application system such as a center pivot irrigation system. Preferably, application to fields of plants and/or crops is made by air spraying, i.e., from an airplane or helicopter, or by land spraying. For example, land spraying may be carried out by using a high flotation applicator equipped with a boom, by a back-pack sprayer or by nurse trucks or tanks. One of skill in the art will recognize that these application methodologies are provided by way of example and that any applicable methods known in the art or developed in the future can be utilized.

This study demonstrated the efficacy of FANA ASOs in reducing the amount of bacterial mRNA, in both in vitro and in vivo experiments, causing a deleterious effect in bacterial density. Specifically, it was shown that the titer of Candidatus Liberibacter asiaticus was decreased, inside D. citri adults, after an orally ingested FANA ASO silenced the expression of CLas LigA gene. This result evidenced the potential of FANA oligos as an environment-friendly alternative for the management of HLB, different than RNAi. Both FANA ASOs and RNAi are post-transcriptional gene silencing technologies that involve the binding of complementary oligonucleotides to target RNA through base paring. However, there are intrinsic characteristics of FANA ASOs that may position them as a better choice than RNAi for controlling HLB. Double stranded RNA (dsRNA) persistency has been a major challenge in RNAi-based pest control. For example, degradation of dsRNA has been reported in hemipteran species such as Lygus lineolaris (Palisot de Beauvois) (Hemiptera: Miridae) and Acyrthosiphon pisum (Harris) (Hemiptera: Aphididae), where the gut pH conditions or possible nuclease enzymes in the saliva and hemolymph digested the exogenous ribonucleic acid. On the other hand, the stability of FANA ASOs to hydrolysis, under acidic and basic conditions, has been shown to be higher than that of DNA or RNA. In addition, previous studies have observed an improved resistance of FANA oligonucleotides to the action of both endo and exonucleases, which was attributed to the chemical modifications in the phosphate backbone and ribose sugar of the oligos. The resistance to degradation of FANA ASOs may lead to a more effective silencing.

In this study, it was demonstrated that a fluorescently labeled FANA oligonucleotide is able to penetrate Drosophila S2 cells, infected with wDi. No transfection agent or molecular conjugation was required for the oligo to enter the insect cells. In a subsequent in vitro experiment, the downregulation of the wDi gyrA gene, by a complementary FANA ASO, indicated that the oligonucleotide penetrated the bacterial cells as well. Self-delivery of FANA ASOs has been previously reported. Souleimanian and colleagues (2012), using a human prostate cancer cell line, observed that FANA ASOs effectively silenced the expression of the Bcl-2 protein, in the absence of any carriers or conjugation. The self-delivery property of FANA oligos may be explained by the phosphorothioate modification in their backbone, which can have a very high affinity for proteins commonly occurring on the cell surface membrane, therefore promoting adsorptive endocytosis.

Oral delivery of LigA-FANA into adult ACPs, caused the degradation of CLas LigA mRNA and reduced the density of the bacterium in the psyllids. The effectiveness of the treatment may be associated with CLas localization inside D. citri. By using qPCR and fluorescence in situ hybridization, Ammar and colleagues (2011) detected the presence of CLas in the alimentary canal (filter chamber and midgut), Malpighian tubules, haemolymph, ovaries, salivary glands, muscle, and fat tissues. Nevertheless, the relative copy number of CLas genomes was significantly higher in the alimentary canal compared with that in the rest of the insect body, suggesting that CLas may replicate or accumulate in the digestive tract of D. citri; Ghanim and colleagues (2017) further demonstrated the bacterium accumulation in the psyllid gut, inside endoplasmic reticulum associated vacuoles. Having CLas concentrated in D. citri digestive tract, could facilitates bacterial penetration and binding to a homologous mRNA by an ingested FANA ASO.

The recommended control strategy for HLB involves the use of insecticides to reduce D. citri populations, supplemented with removal of infected trees to impede bacterial acquisition by uninfected psyllids and the creation of pathogen-free nurseries. Nevertheless, insecticide resistance is a major problem for this approach. Although novel tactics have been successful under greenhouse and field settings, such as thermotherapy, graft-based antibiotics applications or trunk injections of plant activators, there is a pressing need to find better alternatives to boost the in planta HLB control arsenal. Delivery of LigA FANA into infected Citrus trees, through root infusions, caused a significant reduction of CLas titer that persisted for 30 days. Being able to decrease the bacterial pathogen quantity in both its host and vector, demonstrates the potential of FANA ASOs as an alternative method for the management of HLB.

In some aspects, the oligonucleotide sequence is a complement to the sequence of the target insect or bacterial RNA, and the oligonucleotide sequence has at least 80%, 85%, 90%, 95%, 98%, 99%, or more sequence identity to the complementary sequence of the target RNA.

As used herein, the terms “complementary” or “complementarity” are used in reference to polynucleotides (i.e., a sequence of nucleotides) related by the base-pairing rules. For example, the sequence 5′-A-G-T-3′, is complementary to the sequence “‘-T-C-A-5′. Complementarity may be “partial”, in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. As such, a “complement” sequence, as used herein refers to an oligonucleotide sequence have some complementarity to a target RNA or DNA sequence. The complementarity between the target RNA or DNA and the oligonucleotide can be at least 80%, 81%, 82%, 83%, 84%, 85%, 86%, 87%, 88%, 89%, 90%, 91%, 92%, 93%, 94%, 95%, 96%, 97%, 98%, 99%, or 100%.

The term “homology” or “identity” refers to a degree of complementarity. There may be partial homology or complete sequence identity between the oligonucleotide sequence and the complement sequence of the target RNA or DNA. A partially identical sequence is an oligonucleotide that at least partially hybrids to the target RNA or DNA, leading to the formation of partial heteroduplex, and to partial or total degradation of the target RNA or DNA. A completely identical sequence is an oligonucleotide that completely hybrids to the target RNA or DNA, leading to the formation of complete heteroduplex, and to partial or total degradation of the target RNA or DNA.

In many aspects, the target RNA or DNA is prokaryotic or eukaryotic.

In various aspects, the target RNA or DNA is selected from the group consisting of messenger RNA (mRNA), microRNA (miRNA), small interfering (siRNA), antisense RNA (aRNA), short hairpin RNA (shRNA), transfer RNA (tRNA), ribosomal RNA (rRNA), small nuclear RNA (snRNA), double-stranded RNA (dsRNA), locked nucleic acid (LNA), Transfer-messenger RNA (tmRNA), viral RNA, viral DNA, polynucleic acids circular ssDNA, and circular DNA.

As used herein, the term “nucleic acid” refers to polynucleotides such as deoxyribonucleic acid (DNA) or ribonucleic acid (RNA), with RNA being prepared or obtained by the transcription a DNA template. According to the invention, a nucleic acid may be present as a single-stranded or double-stranded and linear or covalently circularly closed molecule.

As used herein, messenger RNA (mRNA) refers to RNA molecules that convey genetic information from DNA to the ribosome, where they specify the amino acid sequence of the protein products of gene expression. In mRNA genetic information is arranged into codons consisting of three base pairs each. Each codon encodes for a specific amino acid, except the stop codons, which terminate protein synthesis. This process of translation of codons into amino acids requires two other types of RNA: Transfer RNA (tRNA) that mediates recognition of the codon and provides the corresponding amino acid, and ribosomal RNA (rRNA), that is the central component of the ribosome's protein-manufacturing machinery.

RNA interference (RNAi) is the phenomenon of gene-specific post-transcriptional silencing by double-stranded RNA oligomers (Elbashir et al., Nature 411: 494-498 (2001); and Caplen et al., Proc. Natl. Acad. Sci. U.S.A. 98: 9742-9747 (2001)). Small inhibitory RNAs (siRNAs), like antisense oligonucleic acids and ribozymes, have the potential to serve as therapeutic agents by reducing the expression of harmful proteins. The double-stranded siRNA is recognized by a protein complex (the RNA induced silencing complex), which strips away one of the strands, facilitates hybridization of the remaining strand to the target mRNA, and then cleaves the target strand. DNA-based vectors capable of generating siRNA within cells are also of interest for the same reason, as are short hairpin RNAs (shRNA) that are efficiently processed to form siRNAs within cells. siRNAs capable of specifically targeting endogenously and exogenously expressed genes have been described (see for example Paddison et al., Proc. Natl. Acad. Sci. U.S.A. 99:1443-1448 (2002); Paddison et al., Genes & Dev. 16: 948-958 (2002); Sui et al., Proc. Natl. Acad. Sci. U.S.A. 8:5515-5520 (2002); and Brummelkamp et al., Science 296: 550-553 (2002)). As used herein, the term “RNAi construct” is a generic term including siRNA, hairpin RNA, and other RNA species which can be cleaved in vivo to form siRNAs. RNAi constructs herein also include expression vectors (also referred to as RNAi expression vectors) capable of giving rise to transcripts which form dsRNAs or hairpin RNAs in cells, and/or transcripts which can be converted into siRNAs in vivo.

microRNA or miRNA refers a small non-coding RNA molecule (containing about 22 nucleotides) found in plants, animals and some viruses, that functions in RNA silencing and post-transcriptional regulation of gene expression. miRNAs function via base-pairing with complementary sequences within mRNA molecules, resulting in the silencing of the target mRNA molecules by cleavage of the mRNA strand into two pieces, by destabilization of the mRNA through shortening of its poly(A) tail, or by rendering less efficient the translation of the mRNA into proteins by ribosomes. miRNAs resemble the small interfering RNAs (siRNAs) of the RNA interference (RNAi) pathway, except miRNAs derive from regions of RNA transcripts that fold back on themselves to form short hairpins, whereas siRNAs derive from longer regions of double-stranded RNA.

As used herein, “aRNA”, “antisense RNA”, “asRNA” and the like refer to single stranded RNA that is complementary to a protein coding mRNA with which it hybridizes, and thereby blocks its translation into protein. asRNAs (which occur naturally) are found in both prokaryotes and eukaryotes and can be classified into short (<200 nucleotides) and long (>200 nucleotides) non-coding RNAs (ncRNAs). The primary function of asRNA is regulating gene expression. asRNAs may also be produced synthetically and have found wide spread use as research tools for gene knockdown. They may also have therapeutic applications.

snRNA (small nuclear RNA), is a class of small RNA molecules that are found within the splicing speckles and Cajal bodies of the cell nucleus in eukaryotic cells. The length of an average snRNA is approximately 150 nucleotides. They are transcribed by either RNA polymerase II or RNA polymerase III. Their primary function is in the processing of pre-messenger RNA (hnRNA) in the nucleus, and they also aid in the regulation of transcription factors (7SK RNA) or RNA polymerase II (B2 RNA), and maintaining the telomeres.

“Double-stranded RNA” or “dsRNA” refers to RNA with two complementary strands, similar to the DNA found in all cells, but with the replacement of thymine by uracil. dsRNA forms the genetic material of some viruses (double-stranded RNA viruses). Double-stranded RNA, such as viral RNA or siRNA, can trigger RNA interference in eukaryotes, as well as interferon response in vertebrates.

The term “LNA” or “locked nucleic acid” also referred to as inaccessible RNA, is a modified RNA nucleotide in which the ribose moiety is modified with an extra bridge connecting the 2′ oxygen and 4′ carbon. The bridge “locks” the ribose in the 3′-endo (North) conformation, which is often found in the A-form duplexes. LNA nucleotides can be mixed with DNA or RNA residues in the oligonucleotide whenever desired and hybridize with DNA or RNA according to Watson-Crick base-pairing rules. The locked ribose conformation enhances base stacking and backbone pre-organization. This significantly increases the hybridization properties (melting temperature) of oligonucleotides.

As used herein, “tmRNA” or “transfer-messenger RNA” refers to a bacterial RNA molecule with dual tRNA-like and messenger RNA-like properties. The tmRNA forms a ribonucleoprotein complex (tmRNP) together with Small Protein B (SmpB), Elongation Factor Tu (EF-Tu), and ribosomal protein S1. In trans-translation, tmRNA and its associated proteins bind to bacterial ribosomes which have stalled in the middle of protein biosynthesis, for example when reaching the end of a messenger RNA which has lost its stop codon. The tmRNA is remarkably versatile: it recycles the stalled ribosome, adds a proteolysis-inducing tag to the unfinished polypeptide, and facilitates the degradation of the aberrant messenger RNA. In the majority of bacteria these functions are carried out by standard one-piece tmRNAs. In other bacterial species, a permuted ssrA gene produces a two-piece tmRNA in which two separate RNA chains are joined by base-pairing.

Viruses are small infectious agents that replicates only inside the living cells of an organism. Viruses have either a DNA or an RNA genome and are called DNA virus or RNA virus, respectively. The genome can also either be single-stranded or double-stranded, leading to single-stranded RNA (ssRNA), single-stranded DNA (ssDNA), double-stranded DNA (dsDNA), or double-stranded RNA (dsRNA); with each individually being able to be either linear or circular. Single-stranded DNA (ssDNA) is usually expanded to double-stranded in infected cells.

In other aspects, the oligonucleotide sequence has at least 80%, 85%, 90%, 95%, 98%, 99%, or more sequence identity to the complementary RNA or DNA sequence.

In an additional embodiment, the present invention provides a method of inhibiting bacterial growth in a plant-chewing or piercing-sucking plant-feeding insect including providing an antisense oligonucleotide to the insect or to its microbiome, wherein the oligonucleotide includes at least one 2′-deoxy-2′-fluoroarabinonucleotide (2′F-ANA)-modified nucleotide and at least one 2′-deoxyribonucleotide, thereby inhibiting bacterial growth.

In a further embodiment, the present invention provides a method of preventing and/or managing of huanglongbing disease in a citrus tree or plant including providing an antisense oligonucleotide to a plant-chewing or piercing-sucking plant-feeding insect or to its microbiome, wherein the oligonucleotide binds to a target RNA or DNA, wherein the oligonucleotide includes at least one 2′-deoxy-2′-fluoroarabinonucleotide (2′F-ANA)-modified nucleotide and at least one 2′-deoxyribonucleotide, and wherein, the target RNA is DNA Gyrase subunit-A, NAD-dependent DNA Ligase-A, DNA-B Helicase C terminal domain, D-Ribulose A (riboflavin), Ribulose A, B, Acetoacetyl-[acyl-carrier protein], Acetylcholinesterase, Amidophosphoribosyltransferase, Acetyl-CoA carboxylase biotin carboxylase subunit, Biotin Ligase, Biotin-(acetyl-CoA carboxylase)-Ligase, Malonyl Coenzyme A, wsp or 16SrRNA, thereby preventing and/or managing huanglongbing disease. In one aspect, the insect harbors of Candidatus Liberibacter asiaticus (CLas). In another aspect, the oligonucleotide inhibits the growth of CLas. In an additional aspect, inhibiting the growth of CLas prevents the transmission of CLas from the insect to the citrus tree or plant.

In a further embodiment, the present invention provides a method of preventing and/or managing of a Liberibacter zebra chip disease in a Solanaceous plant including providing an antisense oligonucleotide to a plant-chewing or piercing-sucking plant-feeding insect or to its microbiome, wherein the oligonucleotide binds to a target RNA or DNA, wherein the oligonucleotide includes at least one 2′-deoxy-2′-fluoroarabinonucleotide (2′F-ANA)-modified nucleotide and at least one 2′-deoxyribonucleotide, and wherein the target RNA is DNA Gyrase subunit-A, NAD-dependent DNA Ligase-A, DNA-B, D-Ribulose A (riboflavin), Ribulose A, B, Acetoacetyl-[acyl-carrier protein], Acetylcholinesterase, Amidophosphoribosyltransferase, Acetyl-CoA carboxylase biotin carboxylase subunit, Biotin Ligase, Biotin-(acetyl-CoA carboxylase)-Ligase, Malonyl Coenzyme A, wsp or 16SrRNA, thereby preventing and/or managing Liberibacter zebra chip disease. In one aspect, the insect harbors of Candidatus Liberibacter solanacearum (CLso). In another aspect, the oligonucleotide inhibits the growth of CLso. In an additional aspect, inhibiting the growth of CLso prevents the transmission of CLso from the insect to the Solanaceous plant.

In one embodiment, the present invention provides an oligonucleotide including at least one 2′-deoxy-2′-fluoroarabinonucleotide (2′F-ANA)-modified nucleotide and at least one 2′-deoxyribonucleotide and having a sequence of SEQ ID NOs: 5-77. In one aspect, the 2′F-ANA-modified nucleotides are positioned according to any of Formulas 1-16. In an additional aspect, the 2′F-ANA-modified nucleotides include internucleotide linkages between nucleotides are phosphodiester bonds, phosphotriester bonds, phosphorothioate bonds (5′O—P(S)O-3O—, 5'S—P(O)O-3′-O—, and 5′O—P(O)O-3'S—), phosphorodithioate bonds, Rp-phosphorothioate bonds, Sp-phosphorothioate bonds, boranophosphate bonds, methylene bonds(methylimino), amide bonds(3′-CH₂—CO—NH-5′ and 3′-CH₂—NH—CO-5′), methylphosphonate bonds, 3′-thioformacetal bonds, (3'S—CH₂—O5′), amide bonds (3′CH₂—C(O)NH-5′), phosphoramidate groups, or a combination thereof. In various aspects, the oligonucleotide is for use in baits, diets, liquids, to target insects, by attraction, or feeding, leading to reduced fitness, fecundity or health of pests, or for the reduction of pathogens, to suppress transmission, or to improve survival of insects considered beneficial to plant health.

The following examples are provided to further illustrate the embodiments of the present invention, but are not intended to limit the scope of the invention. While they are typical of those that might be used, other procedures, methodologies, or techniques known to those skilled in the art may alternatively be used.

### EXAMPLES

### Example 1

### Materials and Methods

FANA ASOs. FANA antisense oligonucleotides were synthesized by AUM Biotech (Philadelphia, PA). For in vitro experiments, a FANA ASO was designed to be complementary to the Wolbachia-Diaphorina, wDi DNA gyrase subunit A gene (gyrA). The CLas NAD-dependent DNA Ligase gene (LigA), was selected as the target gene in the in vivo experiments. wDi was targeted in the in vitro experiments, due to CLas being a fastidious bacterium that cannot be cultured. The sequences of FANA ASOs used in this study are described in Table 1.

Cell culture. Drosophila Schneider 2 (S2) cells were cultured in Schneider's Drosophila medium (Thermo Fisher Scientific, Waltham, MA) supplemented with 10% heat-inactivated fetal bovine serum (FBS; Thermo Fisher Scientific) and 1% penicillin (50U/mL)/streptomycin (50 μg/mL) at 28° C. in a non-humidified incubator. The cells were subcultured to a final density of approximately 1×10⁶ cell/mL every three days. Infection of S2 cells with wDi (S2-wDi cells) was accomplished as described in Bonilla et al., 2018.

Psyllid cultures. Diaphorina citri used in this study were obtained from a culture reared at the University of Florida Citrus Research and Education Center (CREC) (Lake Alfred, FL). The culture was established in 2005 from a field population collected in Polk Co., FL (28.0′ N, 81.9′ W), before the detection of HLB in the state. Psyllids not harboring CLas were maintained on uninfected ‘Pineapple’ sweet orange [Citrus sinensis (L.) Osb. (Rutaceae)] plants in a greenhouse not exposed to insecticides. Insects infected with CLas were collected from a subset of the uninfected D. citri culture but reared instead in CLas-positive ‘Pineapple’ sweet orange plants at a secure quarantine facility. Both psyllid colonies were maintained at 26±1° C., 60-80% relative humidity and a photoperiod of 16:8 (L:D) h. To confirm the absence/presence of the bacterium in the colonies, random subsamples of both plants and insects were tested monthly using a quantitative real-time polymerase chain reaction procedure, previously described (Pelz-Stelinski et al., 2010).

Localization of fluorescently labeled FANA ASOs in S2-wDi cells and D. citri adults. S2-wDi cells were diluted from a density of 8-10×10⁶ cells/mL and 90% viability to a density of 2.75×10⁵ cells/mL. The cells were incubated with 10 μM of a FANA ASO, designed with a scramble sequence and conjugated to Alexa Fluor 647 at its 5′ end, at 28° C. After 24 h, 15 μL of S2-wDi cell suspension was applied into a coverslip previously covered with concanavalin A (Vector Labs, Burlingame, CA) and allowed to attach for one hour. The cells were fixed with 4% paraformaldehyde (Electron Microscopy Sciences, Hatfield, PA) during 45 min, followed by three washes with PBS (Fisher Scientific, Fair Lawn, NJ) of five min each at room temperature. The cells were mounted in microscope slides with VECTASHIELD with DAPI (Vector Labs, Burlingame, CA) and viewed under a confocal microscope (Leica SP8 laser-scanning Confocal).

Leaves of ‘Pineapple’ sweet orange trees grafted on ‘Swingle’ citrumelo rootstocks [Citrus paradisi MacFaden×Poncirus trifoliate (L.) Raf.] were excised and placed in lidless PCR tubes with 200 μL of a FANA ASO solution in water, at a 5 μM concentration, designed with a scramble sequence and tagged with Alexa Fluor 594 at its 5′ end. The leaves were left in the solution for 24 h before placing them inside a 50 mL Falcon tube (Fisher Scientific, Fair Lawn, NJ) with five D. citri adults. After 48 h, the digestive tracts of the psyllids were dissected in PBS, placed on a microscope slide and viewed under a fluorescent microscope (Olympus BX61 Epifluorescence microscope).

### Silencing of Bacterial Genes

Cell culture assays. S2 cells infected with Wolbachia-Diaphorina, wDi, were seeded at a density of 2.75×10⁵ cells/mL, in 24-well culture dishes (Thermo Fisher Scientific), 24 h prior to the treatment with the FANA ASOs. The cells were incubated with 5 μM of either the oligo designed to target the wDi gyrA gene or a scramble sequence oligo, which was used as a negative control. The insect cells were incubated with the FANA ASOs for 7 days at 28° C. Each treatment (wDi gyrA-FANA, [SEQ ID NO: 38], scramble control-FANA [SEQ ID NO: 4], and untreated control) was replicated three times.

Psyllid assays. Leaves (leaf blade and petiole) were collected from uninfected C. paradisi×P. trifoliate trees, washed in 1% bleach for 10 min and rinsed for 5 min by submersion in autoclaved water. The leaves were placed in lidless PCR tubes with 200 μL of either the FANA oligo targeting the [SEQ ID NO: 71], CLas DNA Ligase gen (LigA) or a scramble sequence oligo (negative control). The working concentration of the FANA ASOs solution was 5 PM. After wrapping the tubes tops with Parafilm (American National Can, Neenah, WI), the leaves were placed under artificial light at 28° C., a light dark cycle of 16:8 h and 75% relative humidity, in order to stimulate the absorption of the FANA ASOs solutions. The tubes were filled with nuclease-free water (Thermo Fisher Scientific), after the entire FANA ASO solution was absorbed by the leaves. Treated and untreated leaves were placed inside 50 mL Falcon tubes (Fisher Scientific, Fair Lawn, NJ) and exposed to CLas-infected, ˜three days old, D. citri adults (eight males, eight females) for seven days. Each treatment (LigA-FANA [SEQ ID NO: 71], scramble control-FANA [SEQ ID NO: 4] and untreated control) was replicated three times

Analysis of gene expression. Total RNA extraction from untreated and treated samples (S2-wDi cells or D. citri adults) was performed using Direct-Zol RNA MiniPrep (Zymo Research, Irvine, CA), following the manufacturer's instructions. The concentration and quality of RNA were measured by spectrophotometry (Nanodrop 2000; Thermo Scientific). cDNA was synthesized from total RNA (1 μg) using the High Capacity cDNA Reverse Transcription kit (Thermo Fisher Scientific). Quantitative PCR assays were conducted using a QuantStudio 6 Flex Real-Time PCR Instrument (Thermo Fisher Scientific) and the Syber Green PCR Master Mix (Thermos Fisher Scientific). The data was analyzed using the comparative critical threshold (AACt) method (Livak & Schmittgen, 2001), in which the expression level of the target mRNA in FANA-treated samples was compared to its expression in untreated samples. The wDi gene wsp, which codes for a surface protein, and the CLas 16S ribosomal RNA gene were used as an internal control for cell culture and insect bioassays, respectively. Pairs of primers were designed for the target and the reference genes using Primer3 v. 0.4.0 software (Untergrasser et al., 2012). PCR efficiencies of target and reference genes were confirmed to be within the range of 90-110% for all qPCR assays.

The Shapiro-Wilk normality test and the Levene test of homogeneity of variances were employed to determine the type of distribution for the data obtained in each treatment. T-tests for independent samples or Mann-Whitney U-tests, depending on data distribution, were used to test for significant differences in expression levels (ΔCt values) of the target genes between the experimental and control conditions. P-values less than 0.05 were considered to be statistically significant. The software STATISTICA 13.3 (TIBCO Software Inc, Palo Alto, CA) was used for the data analysis.

Wolbachia-Diaphorina, wDi, viability assay. wDi cells were isolated from S2 cells following the protocol described in Gamston and Rasgon (2007). In brief, the S2 cells were lysed by vortexing the samples with sterile 3 mm borosilicate glass beads at room temperature. The supernatant was centrifuged at 2,500×g (10 min at 4° C.), passed through a filter of 5 μm and centrifuged again at 18,000×g (10 min at 4° C.). The wDi pellet was resuspended in S2 complete media and finally purified using a 2.7 μm filter. Extracted wDi cells were seeded in 96-well culture dishes (Corning Incorporated, Corning, NY), at a density of 300,000 cells/mL, and incubated with 5 μM of either the FANA oligo complementary to the wDi gryA gene [SEQ ID NO: 38] or the scramble control [SEQ ID NO: 4]. Four days after treatment, 800 μL of cell suspension was split into two equivalent samples. One of them was kept untreated and the other one was added with 100 μL of PMA Enhancer for Gram Negative Bacteria (Biotium, Hayward, CA), followed by 5 μL of 2.5 mM propidium monoazide (PMAxx; Biotium). The samples were covered in aluminum foil and incubated for 10 min on a rocker at room temperature. Subsequently, the samples were exposed to intense visible light for 15 min in order to crosslink the propidium monoazide with the bacterial dsDNA (PMA-Lite; Biotium). The bacterial cells were pelleted by centrifugation at 5,000×g for 10 min and used for DNA isolation (DNeasy Blood & Tissue Kit; QUIAGEN, Valencia, CA). For dead cell control samples, 800 μL of untreated wDi suspension was heat inactivated at 95° C. for 5 min and processed as previously described.

For absolute quantification of the wDi copy number, a 250 bp DNA fragment from the wDi gyrA gene was amplified and purified. Ten-fold serial dilution of this fragment were used in qPCR reactions to generate standard curves that allowed conversion of delta threshold cycle values (Ct with PMA−Ct without PMA) into an estimate of alive wDi genome copy number. Quantitative PCR assays were conducted using a QuantStudio 6 Flex Real-Time PCR Instrument (Thermo Fisher Scientific) and the Syber Green PCR Master Mix (Thermo Fisher Scientific). The standard curve obtained for wDi was (y=−3.594x+3.796; R²=0.99). One-way analysis of variance (ANOVA) was used to evaluate the dissimilarities in wDi copy number between the treatments. Means were separated by Tukey's honest significant difference test. P-values less than 0.05 were considered statistically significant.

CLas quantification in psyllids. A group of infected D. citri teneral adults (eight males, eight females) were fed an artificial diet solution, consisting of 17% (w:v) sucrose in autoclaved distilled water and 0.5% green food dye (McCormick & Company, Baltimore, MD). The FANA oligo [SEQ ID NO: 71] targeting the CLas LigA gene was diluted into the artificial diet to a final concentration of 5 μM. The artificial feeding system consisted in a bottomless petri dish (35 mm×10 mm), two pieces of thinly stretched Parafilm (American National Can) and a filter paper disc (Russell and Pelz-Stelinski, 2015); the artificial diet (300 μL) was dispensed on the filter paper located between the Parafilm layers. Feeding assays were held for seven days, in an environmental incubator at 16:8 h light:dark cycle, 27±2° C., and 60-65% relative humidity. The FANA scramble control, at the same final concentration in feeding solution, was used as a negative control. Untreated psyllids were exposed to only sucrose solution. Three replicates were conducted for each treatment.

DNA was extracted from each sample using the DNeasy Blood and Tissue Kit (QIAGEN) and its concentration quantified by spectrophotometry (Nanodrop 2000; Thermo Fisher Scientific). DNA samples were diluted to 50 ng μL⁻¹ for subsequent qPCR analysis. A multiplex TaqMan qPCR assay was performed using probe and primers targeting CLas 16S rRNA gene and D. citri wingless (Wg) gene (Table S1). All qPCR reactions were performed on a QuantStudio 6 Flex Real-Time PCR Instrument (Thermo Fisher Scientific) using the PerfeCta qPCR ToughMix, Low ROX (Quanta BioSciences, Gaithersburg, MD). Absolute quantification of CLas copy number was calculated using dilution series of a plasmid containing the target region of the CLas 16S rRNA gene, as described in Chu et al. (2016). The standard curve obtained for CLas was (y=−3.286x+10.338; R²=0.99). CLas copy number was divided by the Wg gene copy number in the same sample. One-way analysis of variance (ANOVA) was used to evaluate the differences in CLas copy number between the treatments. Means were separated by Tukey's honest significant difference test. P-values less than 0.05 were considered statistically significant.

FANA ASO root infusions and CLas quantification in planta. Two to three years-old Citrus paradisi×Poncirus trifoliate, not treated with systemic insecticides, were used for plant assays. The trees were inoculated with CLas by exposing them to infected ACPs for a month. After the inoculation access period, all developmental stages of ACP were eliminated from the trees by an insecticide treatment. The plants were maintained in a greenhouse for four months to allow for systemic infection.

The initial CLas titer (TO) of each tree was calculated by collecting three leaves per plant and extracting their genomic DNA, followed by quantitative real-time PCR. The Citrus trees were then treated with the [SEQ ID NO: 1] LigA FANA ASO by root infusion. For root infusions, the main root system of the trees was gently scraped under water with a razor blade and fitted into a PVC tube (1 m long, 6 mm diameter). The tubes were filled with 5 mL of either a 5 μM FANA ASO solution, a 5 mg/mL streptomycin sulfate solution (Thermo Fisher Scientific) or water (untreated control). Tubes were filled with water 24 hrs post treatment. From each tree, three leaves were removed from similar locations as the T0 samples at two, seven, 14 and 30 days post treatment, to monitor the effect of the FANA ASO on the plants CLas titer. Five trees were used per treatment.

Leaf DNA was extracted as described in Pelz-Stelinski et al. (2010) and diluted to 15 ng μL⁻¹. A multiplex TaqMan qPCR assay was performed as stated before, using probe and primers targeting CLas 16S rRNA gene and the citrus mitochondrial cytochrome oxidase gene (Cox) as internal control for DNA extractions. CLas copy number was quantified as previously reported (Chu et al., 2016). The standard curve obtained for CLas in the plant experiments was (y=−3.312x+11.763; R²=˜0.99). The treatments effect was expressed as percent change in CLas titer [(mean titer after treatment−mean titer prior to treatment)/mean titer prior to treatment]×100 (Hu et al., 2018). One-way analysis of variance (ANOVA) was used to compare the plants initial and final CLas copy number within the treatments. Means were separated by Fisher's least significant difference (LSD) test, considering P-values equal or less than 0.05 as statistically significant.

TABLE 3
qPCR primers

| TARGET | TARGET | PRIMER/PROBE |
|---|---|---|
| SPECIES | GENE | SEQUENCE (5′- 3′) |
| Wolbachia | gyrA | ACTAACATCATGCCGCTTCC |
| | | (SEQ ID NO: 78) |
| | | ATTGACGCACATCGCTTACA |
| | | (SEQ ID NO: 79) |
| Wolbachia | wsp | AGGGCTTTACTCAAAATTGG |
| | | (SEQ ID NO: 80) |
| | | CACCAACGTATGGAGTGATAGG |
| | | (SEQ ID NO: 81) |
| Candidatus | LigA | ATTCCAATCGAGGCATTGAG |
| Liberibacter | | (SEQ ID NO: 82) |
| asiaticus | | TCAGAGCGTGCTAAATCAGG |
| | | (SEQ ID NO: 83) |
| Candidatus | 16SrRNA | TCGAGCGCGTATGCAATACG |
| Liberibacter | | (SEQ ID NO: 84) |
| asiaticus | | GCGTTATCCCGTAGAAAAAGGTAG |
| | | (SEQ ID NO: 85) |
| | | AGACGGGTGAGTAACGCG |
| | | (SEQ ID NO: 86) |
| Diaphorina | Wingless | GCTCTCAAAGATCGGTTTGACGG |
| citri | (Wg) | (SEQ ID NO: 87) |
| | | GCTGCCACGAACGTTACCTTC |
| | | (SEQ ID NO: 88) |
| | | TTACTGACCATCACTCTGGACGC |
| | | (SEQ ID NO: 89) |
| Citrus spp | Cox | GTATGCCACGTCGCATTCCAGA |
| | | (SEQ ID NO: 90) |
| | | GCCAAAACTGCTAAGGGCATTC |
| | | (SEQ ID NO: 91) |
| | | ATCCAGATGCTTACGCTGG |
| | | (SEQ ID NO: 92) |

TABLE 4

| Gene | Sequences |
|---|---|
| | (SEQ ID NO: 93) |
| QXDO01000001.1: | ATGAAAAAACGTCAATTGATTCCAATCGAGGCATTGAGTATCGAACAAGCTAGAAAAG |
| c1165055-1162860 | AATTATCGTCTCTTGAACAAGAGATATCATATCATGATGATTGTTATTATCAGTATTC |
| Candidatus | AAAACCTATAATCTTAGATTCTGAATATGACGCATTAAAAAGACGTTGTGATGCTATT |
| Liberibacter | GCACATGCTTTTCCTGATTTAGCACGCTCTGAGGATCCTAATGGGCCTCTGAATAAAA |
| asiaticus strain | TAGGGGGAGAAATGAACCTCTCCTTATTTAAAGTGACTCATTCTATCCCAACATTATC |
| YNJS7C contig1, | ATTAGAAAAGATTTGGAATATCGAAGATCTTCGTAAGTTCATCACTAGAATTTATCGT |
| whole genome | TTTTGGCAAAAAAAAGATGATAACAGCATTTTTTTTACTCTGGAGCCTAAAATTGATG |
| shotgun sequence | GGACAATGATTACTATACGATATGAAAAAGGAAAGTTTGTTTATGCTGCTTTGCGTGG |
| NAD-dependent | TGACGGTCATAGCGGAGAGGATGTTAGTGCTTGTATTCGTGCTATTCCGACAATTCCT |
| DNA ligase A | CGTGTTTTATCATTGAATATTCCAGAAATTATAGAAGTGCGTGGTGAAGTTTACATCT |
| | CAAAAAATGATTTTCTTGCGCTTAATGATGAAATGATTGCAGTCGGCCGAAAACCCTA |
| | TGCTAATCCTAGAAACGCTGCTTCTGGCATATTACGTCGTTTAAATCCTACGGATATT |
| | ACTCGGTATTTGAATTTTTTTGTGCATGGATTAGGAAAAACATCTGAGATATTCGCAA |
| | AAGGTCAGTATGAAATGCTACAAAAAATGCGTTGTCTAGGCTTTCCTGTTAATAACGG |
| | AGTTCGACAAGCGAATACTTTTCACGGTATCGTATCCTATTATAATGAAATGAAATCA |
| | ATACGTTCTACTCTTCCATATGATATAGATGGATTGGTATATAAGGTCGATGAATTTT |
| | CITTACAAAAACAATTAGGAGAACGAGCTCGAAGCCCACGTTGGATGATATCGCATAA |
| | ATTTGCAGAGAAACAAGCATCTACGCGTCTTTTAGATATTGATATTCAAATTGGTCGT |
| | ACTGGTATTCTCACGCCGGTAGCTCGTCTTGAACCTGTTAACATAGGAGGTGCTTTAA |
| | TCACTAATGCAACATTGCATAATGAAGATTACATCAAAGGATTGGATGCATCTGGTAA |
| | AGTAATGCGTGGAGGAAGGGATATTCGTGTAGGGGATAGGGTCTTGGTCAAAAGAGCC |
| | GGAGAGGTGATTCCTAAAGTIGTGGATATTATTGTCAATGAAAGACATCCTGATGCTC |
| | AACCGTTTTCTTTTCCTTCTTTTTGTCCTATCTGTCAGAGTCGAGTAGTGCGTGATTT |
| | AAATCCGAAAACAGGAAAACTTGTTGCTGCCCATCGTTGTACGGGAGGACTTGCGTGC |
| | 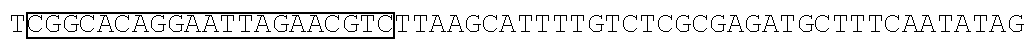 |
| | AAGGTTTAGGAAAACAACAATTGGATTTCTTCTTTAAATCAGAAGATCCTGCTTTTTC |
| | TATTAGGATTCCTGCCGATATTTTTACATTGCAACGTCGTCAACAAACTTCTACTACA |
| | AAGATTGAGAATATTCTAGGTTTTGGCGATGTTAGTGTCACCAATCTTTACGATTCTA |
| | TTAATAAAAGACGCAATATTTCTTTGGAAAGATTTATATTTTCCTTAGGTATTCGGCA |
| | TGTTGGCGCAGAAATTGCCAGATCTTTGGCGAAATATTATCTCTCATATCAAAATTTT |
| | GCACAAGAAATCAACAACATTATTGCACATAACAATGATGATTGGCTGTCTTTAATAA |
| | GAGTTCCTCTAGTAGGAGATATAATAGCGCAGGCTATTGTAGAATTTTATCAGAATCC |
| | TAGGAATATTTGTGCTGTAGAAGCACTGTTAAAAGAAGTATCTCCTTCTATTGGGCGT |
| | CACGAAAAAAATGTCAGCTCTGAGATCGAAAATAAGAAATTAGTATTTACCGGAACCC |
| | TGCAGAAAATTCAACGCCATAAAGCACAAGAATATGTGACTCAGTTAGGTGCTGTTGT |
| | TTCTGCTATTCTTTCTCGTAAAACTGATATTATTATTGTTGGTGATAATCCTGGTTCC |
| | AAGTTAGAGAAGGCACAACAACTAGGTGTTAAAATAATGAACGAAGAGCAATTTTTAT |
| | TTCTATTACAACAGTATAATACAACGTTGCGTATCCACGACGATGACTAA |
| | (SEQ ID NO: 94) |
| gil436410858lgbIA | CAAAGTATAGAAAATGCAAGACAATATAGTACCAGTTTCGATAGTAAAAGAGCTAGAA |
| MZJ01000064.11 | GATTCTTATCTCTCCTACGCAATGAGTGTAATCATAAGCCGAGCTATACCTGACGTGC |
| Wolbachia | GAGATGGATTTAAACCTGTACATAGGCGTATATTATATGCAATGTCAAGAGCTGGGTT |
| endosymbiont of | CGATGCAGGTAAGCCATATAAAAAGGCAGCTCGTATAGTTGGAGATGTAATGGGAAAA |
| Diaphorina citri | TATCACCCACATGGTGATGCAGCTATTTACGATTCCTTGGTCAGGATGGCTCAAGATT |
| Contig54.1, whole | TTTCTCTTCTTTTACCACTTATTGATGGGCAAGGTAACTTTGGTTCGATAGATGGAGA |
| genome shotgun | TCCGCCAGCTTCAATGCGATATACAGAAGCAAGGCTCCACAGGGTGTCGCATTTTTTA |
| sequence | CTGAATGATATTGATGAAGATACAGTTGATTTTAGGGCAAACTACGACGGAAATGAAA |
| DNA gyrase | CGGAGCCTGTCGTATTGCCGGCAGAATTTCCTAATCTGTTGGTAAACGGTGGAAGTGG |
| subunit A | 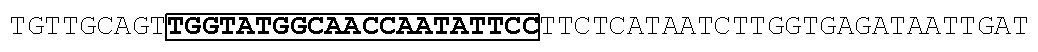 |
| | GCTTGCATGTTATATATAGATAACCCTGAAGTTACTCTAGATGAGTTGCTTGAAGTAA |
| | TACCAGGGCCGGATTTTCCAACGGGAGGAATAATTCTTGGCAGATCTGGAATAAGATC |
| | AGCGTTTGCTAAAGGCCGAGGTTCAATTGTCGTTCAGGGTAAAACTCATATAGAAAAT |
| | CTGCCACAAGATAGACAAGCAATAGTTATTGATGAAATACCTTACCAAGTAAATAAAG |
| | TAAAATTAATTGAGAAAATAGGCGAGCTCGCAAAAGAAAAAAGAATTGAGGGCATAAC |
| | AGAGATTAGGGATGAGTCTGATAAGTCTGGTATTAGAGTAGTAATTGATCTCAAAAAA |
| | AACACTGAAGCAGATTTTATATTGAATCAAATACTAGGACTTACTCCTCTGAGAAGTA |
| | GCTTTAGTGTTAATACTTTAGTTCTGAACAACAACAGGCCTGCTTTGATGTCATTGAA |
| | AGAAATCATAGCTGCTTTTATTGACTTTAGAAAAGAAGTACTAATCAGGAGAACAGAG |
| | TTTCGTCTAAAAAAGACGAGAGAAAAAGCTCATATATACATAGGGCTCTACATTGCGG |
| | TCCTCAGCATAGATGAAGTGATAAAAATCATCCGTGGTGCAAAAGATCCTGAAGAAGC |
| | AAGCAGAGAGCTTTTAAACAAGGAATGGAAAACCTCAGCTGAAATAAACACGATTATT |
| | GAATTAATCTCAGACAGTATGAGCTTTTTGAAAGATGGAGTGTATAGATTAACTGAGC |
| | TGCAAACAAAAGCTATTCTTGATATGAAATTGCAACGCTTAACAGGCCTTGAAAAAAG |
| | CAAATTAGAAAATGAGTTAAATTCAATGCTTAGCCTGATAAAAGAATATATCGCTTTT |
| | CTTGGCTCAGAAGAGAAATTGATGCAAGAAATAAAAAACAATTTACAAGAAATAAAGA |
| | ATAGATTTGCCGTACCTCGAAGAACTTCAATAGAAGAATCAGACATGGACATTGAAGC |
| | TGAAGATCTAATTCCACAAGAAGATATGGTAGTGACCGTAACTATGAATGGTTACATC |
| | AAACGTGTGAAGCTCTCTCACTATAGAACCCAGCATCGTGGTGGAAAAGGAAAGCTAG |
| | GACAGGGATTAAAAGAGGAGGACGTAATCACAAAATTATTTGTTGGAAATACTCATAC |
| | TAGCCTTTTATTCTTTTCTAATATCGGTCGAGTTTACAGATTAAAAGTTTACAAACTG |
| | CCTCTTGCAGAGCCAACTGCACGTGGAAGAGCGCTTGTTAATATATTCCCGCTTAGCG |
| | ATGGTGAAACTATAACTAACATCATGCCGCTTCCAAGTGAGAATGACGAAAATCAAAA |
| | TATAGTTTTTGCTACTGCTCATGGGAACATAAGGCGTAACTCCTTAGCGGATTTTCAC |
| | TATATTCCAAGCAATGGAAAAATAGCAATAAAGCTCGATGAAGGGGATAAATTAGTGT |
| | CAGTTAAAGTATGTAGCGAAATTGATCACGTTCTACTTTCAACAACGCTTGGAAAAAG |
| | CATTAGATTTGTTGTAAGCGATGTGCGTCAATTTAAGAGTCGCAATTCAGATGGAGTA |
| | AGAGGTATCAAACTTGCAAAAAATGACAGCGTGATATCCACGACCATACTAAACGGCA |
| | TAGGTATTGCAACAGAAACAAAAGAACTTTACTTAAGAGTTCCGCTTGCAAAAAGGAT |
| | GGAAGCTGCAACCAATAACTCAATTGATTCTAAATCAGAAAAAACTTTGAATGATTTA |
| | GGAATAAAGAACGAATTATTCTCAAAACTCGCACTTAATGAGGAATTCATATTAACTA |
| | TTACTGAAAATGGCTTTGGTAAAAGAACTTCTGCATATGAATATAGGGTAACAGGTAG |
| | AGGTGGTGTTGGTATTACAAATATCCTTACTACTAGTAGAAATGGCAATGTTGTTGCT |
| | AGTTTTCCAGTTGAGCAGGATGACAACATAATGCTTATTACAGATAAAGGAAAGTTAA |
| | TTCGCATTTCGGTTAATGAAATTAGAATTGCAGGACGCAGCACTCAGGGAGTTACTCT |
| | GTTTAAAACAGAAAATAAAGAAAAGGTGGTATCGGTAGCAAAAATTGAAGATCCTAGC |
| | TCCACTGAAGAGAATATGCCTGAGGTTGAAAGTGCTACTTCTTCTTAATTATGTAGCA |
| | AAGGCTAAGCAAACAGTGCAAATTTCTATTGATTAATCTATAATATTTAAATTAAAAT |
| | ATATGTCTATGTTCAAGTAGAGATTGATGGATAAGAAGTTAACTAAAAATAAAAAATC |
| | ATTGACGAATGAGAAGAATAAAAAAACATCTCTTGTTAAAGATTTTGCCAAAAATAAG |
| | AGTAGAAGAGATTTTATCACATTAACTACGTGTGCCATGGCAGGTATAGGAGCTGCAA |
| | GCGGTTTTTGGCCATTGGTTAAGTCTATGAATCCTTCTGCTGAGGTTTTAGCAATGTC |
| | TACGGTTGAGGTTAATCTATCTGACATTCAAGAAGGACAAGGAAAAAAAGTAAAATGG |
| | CAAGGT |
| | (SEQ ID NO: 95) |
| >JWHA01000001.1 | ACCTAAAATTGATCCTATAGATCCTGATGTACTCCACCAAAAATTTCAAGAACACTTT |
| Candidatus | ACACAATCATTATTACAGAAATATTTCTATGAATCACTTACATTCTAATTATCCGATA |
| Liberibacter | TGGCATCCTTTTGTTCAACATTTTTGTAATCCTACTTTTAAAAAGATTATTAAGACAG |
| asiaticus strain | AAGGATCTTATCTTATTGATGAAAATCACGATAAGATTATTGATGCCATTTCTTCTTG |
| FL17 contig1, | GTGGGTTATTACCCATGGACATCGTCATCCTATAATTATGAATGCAATACGATCTGCA |
| genome shotgun | TCTGAAAACTATGATCAAATAATTTTTTCCGAATATACTCACGAACCCGCTGAACAAC |
| sequence Gyrase_A | TTGCAAGATTACTCATTAAAATTGCACCTCTAGGGTTACAATACGTTTTTTTTTCTGA |
| | TAGTGGCTCTACTTCTATAGAAGTAGCACTCAAAATGGCCCTAGGGTTTTTTAATAAT |
| | AATAACATGCCACGTAACAAAATTGTCGTCATGGAACATGGATACCATGGAGATACAA |
| | TTGGATCTATGTCAGTTAGTATGCGTGGAGCATTTAATAATTCTTATGAACCTCTACT |
| | GTTCGATGTAGAAACCATTCCTTTTCCACATCATAATGAAGAACAAAAAACCCTTGAT |
| | GATCTTGAAAAACACTGTAGTACCCAAAAAATAGCTGCTTTCTTGGTAGAACCATTAA |
| | TACTTGGTGCGGGCGGTATGCAAACCTATCGTCCATACATTCTAAAAGAATTTATGCG |
| | TATAACAAAAAAATATGATACACTTCTAATTGCAGATGAAGTCATGACTGGATGGGGA |
| | AGGACAGGGAAAATTTTTGCTTGTAATCATGCAGATATTACTCCTGATATTCTTTGTT |
| | TATCTAAAGGAATTACAGGAGGTGCATTACCATTAGCAGCTACACTTTGCTCTGAAGA |
| | AATTTTTGATTCTCACTTATCTTCTGATAAGAAGAAAACATTTTTTCATTCCAGCTCG |
| | TATACTGCTAATCCTATTGCTTGTGCGGCAGCACTTGCTAACCTTCAAATATGGGAAA |
| | AAGAACCCGTAATAGAGCGTATAAAAAATTTAGAAAGAATGCATGCTGATAGGTTGTC |
| | ATTACTTAAATCGAACAAAAAATTTGTCAATATAAGGCAAATGGGCACAATTGTAGCG |
| | CTAGATTTTAATGTTCAAAACAAAGGTTATTTTTTTGATAAAAATGCTAAATTAAAAA |
| | ATTTTTTTCGCGAAAAAAAGATTCTTATTCGCCCTTTAGGACATATAATGTATCTTAT |
| | GCCCCCATATTGTATAACTGCTACTGATCTTGATCACATATATAATACAATTAACGAA |
| | GCTGTTGATTTTTTGGAGTTATAATTGATAAAAAGTTCTAGCTGCATTCTCGGATTCG |
| | GACATGCTGTTCCTAACCAATGCATTCATAATTCTATAATTGAAAGACACCTAAATCT |
| | TAAGCTAGGTGTTATAGAACAAAAAACTGGTATTAAATATCGATATTGGGCTGAAAAA |
| | CACGAAACACTAACTGATATTGCAATACAAGCAGGAGATATAGCTTTACGAAATGCTA |
| | ATATCAAAAAGGATAGCATTTCTTTAACACTTCTTGCTACTTCAACACCTGATCACTT |
| | ATTACCACCATCTTCTCCTCTCATCACTCATCGCTTGGGACTTATGCAATCTGGAG |
| | (SEQ ID NO: 96) |
| >gil527317437:3284 | ATGAATCAATTTGCTAAAGAAGTAATTTCTATTTTACTTGAAGAGGAAATGTGTAAAA |
| 33-331042 | GTTATCTTGATTATGCGATGAGTGTAATTGTTGGGAGAGCATTACCAGATGTGCGGGA |
| Candidatus | TGGATTAAAACCAGTTCATAGACGTGTATTATTTGCTATGCATGAAATGAATAATTTA |
| Profftella | TGGAATCGTCCTTTTGTAAAATGTGCACGAGTTGTTGGGGAAACTATGGGAAAATACC |
| armatura, | ATCCTCATGGTGATGTATCAATTTATGATGCTTTAGTAAGAATGGCACAATCATTTTC |
| GyrA_complete | TTTACGTTGCACATTAGTAGATGGACAAGGTAATTTTGGTTCTATAGATGGGGATAGT |
| gyrase-A | GCTGCGGCAATGCGTTATACTGAATGTCGTTTAAATAAAATTTCTAATGAATTACTTA |
| | TTGATTTAGATAAAGAAACTATTAATTTTATTTCAAATTATGATGGAAAAGAAAAAGA |
| | ACCATCTGTATTACCAACACGTATTCCAAATTTACTAATAAATGGTTCTTCIGGTATT |
| | GCTGTTGGTATGGCGACTAACATACCTCCTCATAATTTAACAGAAGTTATTGATGGTG |
| | TATTATATGTTTTACATAATCCAGAATGCTCAATTAATGATTTAATCAAGATAATTCC |
| | AGCTCCTGATTTTCCAACAGCAGGTATTATTTATGGATTATCTAGTGTTCATGATGGC |
| | TATTATACTGGAAAAGGTCGTGTAATAATACGAGCCAAAACTCATATAGAAGAATTTA |
| | ATAGAGAAAATCGCACAGCAATTATTATTGATGAACTTCCATATCAAGTTAATAAAAA |
| | ATCATTATTAGAAAAAATTTCTCAATTAGTTAAGGAAAAAAAATTAGAATGTATTTCT |
| | AATTTACGAGATGAATCAGATAAATCTGGGATGCGTATAGTTATTGAATTGAAGAGAA |
| | ATGAAATACCAGAAATAGTATTAAATAAATTATATAAACAAACTCAGTTACAAAATAC |
| | CTTTGGTATGAATATGTTAGCATTAGTTAATGGACAGCCTAAATTATTAAACCTAAAA |
| | GAAATACTTTATTATTTTATTTTACATCGTAAAGAAGTAATAAAACGAAGGACTATTT |
| | TTGAATTACGTAAAGCGC |
| | (SEQ ID NO: 97) |
| >gil527317437:3284 | ATGAATCAATTTGCTAAAGAAGTAATTTCTATTTTACTTGAAGAGGAAATGTGTAAAA |
| 33-331042 | GTTATCTTGATTATGCGATGAGTGTAATTGTTGGGAGAGCATTACCAGATGTGCGGGA |
| Candidatus | TGGATTAAAACCAGTTCATAGACGTGTATTATTTGCTATGCATGAAATGAATAATTTA |
| Profftella | TGGAATCGTCCTTTTGTAAAATGTGCACGAGTTGTTGGGGAAACTATGGGAAAATACC |
| armatura, | ATCCTCATGGTGATGTATCAATTTATGATGCTTTAGTAAGAATGGCACAATCATTTTC |
| GyrA_complete | TTTACGTTGCACATTAGTAGATGGACAAGGTAATTTTGGTTCTATAGATGGGGATAGT |
| | GCTGCGGCAATGCGTTATACTGAATGTCGTTTAAATAAAATTTCTAATGAATTACTTA |
| | TTGATTTAGATAAAGAAACTATTAATTTTATTTCAAATTATGATGGAAAAGAAAAAGA |
| | ACCATCTGTATTACCAACACGTATTCCAAATTTACTAATAAATGGTTCTTCTGGTATT |
| | GCTGTTGGTATGGCGACTAACATACCTCCTCATAATTTAACAGAAGTTATTGATGGTG |
| | TATTATATGTTTTACATAATCCAGAATGCTCAATTAATGATTTAATCAAGATAATTCC |
| | AGCTCCTGATTTTCCAACAGCAGGTATTATTTATGGATTATCTAGTGTTCATGATGGC |
| | TATT |
| | (SEQ ID NO: 98) |
| >WP_015589386.1 | ATGCTAGATGAAATGCGTGAGGAATGTGGGGTATTTGGCATAAGTTGCAACCAAAGTG |
| amidophospho- | CTGCTTTTAACTCTATACTTGCTCTCCACGCTTTGCAGCATAGAGGTCAAGAGTCTTT |
| ribosyltransferase | TGGCGTAGTAACCAGTAACAACGATAAGCTGCACTCTTATCACTTTCAAGGTCAAGTG |
| [Wolbachia | AGCAGCGTATTTGATGATATAGATGAAATAAAGAAATCCTTACCTGGAGATTGTGCAA |
| endosymbiont of | TAGGTCATGTTCGATACTCAACAAGTGGTAATAAGTTTGGAGTGCAGCCCATGTTTAG |
| Drosophila | CAAAAGTGGCAAATTTGGGGATTTCGCCATAGCACATAATGGTAATTTAATTAATATT |
| simulans. | TCTCCGATACGCGAACAATTGATTAAACAAGAGTGCGTTTTTCAGTCAGATATTGATA |
| Fruit FLY | CAGAAGTAGTAGTGCATCTGACAGCAAGCGGTGAAAAAGATAGCTTTTTAGAGAGTTT |
| | TATATATGCATTAAAGCAAATACAAGGTGCTTATTCTTTTGTTGCAATCAATCAAGAA |
| | GTAGTTATTGGAGTACGTGATTCATCTGGAATCAGGCCTCTTGTTTTAGGAAAGTTAA |
| | ACGGTTCTTACGTTCTTGCATCTGAAACTTGTGCTCTTGATATAATAAATGCAGAGTT |
| | TGTTAGAGAAATAGAACCAGGCGAATTAGTTACCATCAGTTCGGGTAGTAAGCTAGCC |
| | TCAATGTTTCCTTTCCCACAGCAAAAATCAAGTTTTTGTATTTTTGAATACGTTTATT |
| | TTTCAAGACCAGACAGCATAATGGAGAATAGGTCTATATACGATACAAGAAAAGAAAT |
| | AGGAAGAATACTTGCAGAAGAGAGCCCACCAAAAAACAATGTAGATATGGTTGTACCA |
| | ATACCTGATTCTGGAATACCTGCAGCTATTGGATATGCAAAGCATTCAGGATTACCTA |
| | TGGAACTGGGAATAATTCGTAATCATTACATAGGAAGAACTTTTATACAACCAACCGC |
| | TGAAGTACGTAAAGTTAGAATAAAATTAAAATTCAATGCTAATAAGCACACTTTGAAA |
| | GGTAAAAACATAATTTTAATAGATGATAGTATAGTGCGTGGTAGTACGTTAACAAATA |
| | TAATAGTTATGCTAAAGGATGCAGGAGTAAAAGAGATTCACCTCAAAATTTCAAGCCC |
| | ACCAATTAAGCATTCTTGTTTTTATGGAATAGATACGCCAGAGTGCAAAGATTTAATT |
| | GCTGCAAATAAATCTGTAGAGGAAATTAAGGAAGTTATAGGAGTAGATAGCCTAGCCT |
| | TCTTGAGTATTGATGGATTATATCAGGCTGTTAAAGGAGAAGTGCGTAACAATGCTAC |
| | ACCACAATATTGTGATGCTTGTTTCACTGGTGATTATCCAATTGGCAAATAG |
| | (SEQ ID NO: 99) |
| >CP002371.1 | CCTAAAATTGATCCTATGGATCCTGATATACTTCATCAGAATTTTCACAAAAATTTTG |
| Candidatus | CACAATCACTATTAAAGAAAAATGTTTATGAATAATTCAAACTATAAGTTTCCGATAT |
| Liberibacter | GGCATCCTTTTGTTCAACATTTTTTCAATCCTACTTTTAACAAGATTATTAAGACACA |
| solanacearum | AGGAACTTATCTTATTGATGAAGATCACAATAAGATTATCGATGCAATTTCTTCTTGG |
| Reverse | TGGGTTATTACGCATGGGCATTGTTATCCAAAAATCATGCGTGCCATACGATCTGCAT |
| Complement | CTAAAAATTATGATCAAATAATTTTCGCTGAATATACCCATGAACCCGCTGAAAAACT |
| Gyrase A region. | TGCAAAATCACTGATTAAAATTGTACCCCCAGGGTTACAATATGTTTTTTTCTCTGAT |
| from | AGTGGTTCTACTTCCGTAGAAGTAGCACTCAAAATGGCGCTAGGTTTTTTTTATAATA |
| mRNA from | ATAATATAGAACGTCATAAAATCGTTGTCATGGAACATGGATATCATGGTGATACAAT |
| genome DNA data | CGGAGCTATGTCAGTTGGAATGCGTGGAGCATTTAATAATCCTTATCAACCTCTATTA |
| | TTTGATGTAGAAAAAATTCCGTTTCCATATCCTCATTCAGAACAAGAAACTATTGATT |
| | GTCTTGAACAACACTGTCGCACCAAAAAGATTGCTGCTTTACTGATAGAACCACTAAT |
| | ACTTGGTGCAGGAGGTATGAAAACCTATCGTCCATATCTTCTAAAAGAATTTATACGG |
| | ATAACAAAAAAATATGATACCCTTCTCATTGCAGATGAAGTGATGACTGGATGGGGAA |
| | GAACGGGCGAACTTTTTGCTTGTAATCATGCAAATATTACCCCAGACATACTTTGTTT |
| | ATCTAAGGGAATAACAGGAGGTGCATTACCACTAGCGGCTACACTGTGCTCTGAAGAA |
| | ATTTTTGATTCTCATGTATCTTCTGACCCTAGAAAAACATTTTTTCATTCTAGTTCTT |
| | ATACTGCTAATCCTATTGCTTGTGCAGCAGCACTTGCTAATCTTAAAATATGGCAAAA |
| | AGAACCAGTAATGGAACGCATTAAAAATTTAGAAAAAATGCATGCTGATAAGTTGCTT |
| | TTATTCCAATCAAATAAAAAATTTATTAATATCAGACAAATAGGAACAATTGTAGCGC |
| | TTGATTTAAATGTTCAAAATAAAGGTTATTTTTTTAAAAAAAATGCAGAATTAAAAAA |
| | ATTTTTCCGCGAAAAAAATATTCTTATCCGCCCTTTAGGGAATGTAATATACCTCATG |
| | CCTCCTTACTGTATAACATCAAGTGATATTGATCATATATATAATACAATTAATGAAA |
| | CTGTTGATTTTTTGGAGTTATAGTTGATGAAAAGTTCTAGITGTATTCTTGGATTCGG |
| | ACATTCTGTTCCTAGCCAATGCATTCACAATTCGATAATTGAAAGACACCTAAATCTT |
| | AAATCTGGAATTATAGAAAAAAAAACTGGTATTAAATATCGATATTGGGCAGGAAAAA |
| | ACGAAACGCTAACTGATATTGCAATACAAGCAGGAGATATAGCCTTACGCAACGCTAA |
| | TATTAACAAGGATAGCATTGCTTTAACACTTCTTGCCACTTCAACTCCTGATCACTTA |
| | TTACCACCATCATCTCCTGTAGTTACCCATCGCCTTGGACTTTTAAAATCCGGAG |
| | (SEQ ID NO: 100) |
| >CP001677.5:2264 | ATGATTAATACAGCTCAGGATATCATTTTATCTATTCCAAAAGAAGATTCCTCTCAAT |
| 55-227969 | ATCGTGAATCTCCCAATAACATTGAAGCAGAACAAGCTCTTTTAGGAGCTATCCTTGT |
| Candidatus | GAATAATGAGGCTTTTTATCGAGTATCAGATTTCTTAAAACCGTTGCATTTTTTCGAA |
| Liberibacter | GCGATTCACCAGAAAATTTTCGAAATAATGGGTAAGTTGGTTCACATGGGAAAAACAG |
| asiaticus str.psy62 | CTAATCCCGTTACGGTTAAAACTTTCTTATCTGATCAAGATATGTTAGGAGAGCTAAC |
| DnaB-like | AGTACCACAGTATTTAGCGCGTCTAGCTTCCGAAGCAGTTTCAATAATAAATACCGAA |
| Helicase C. | GATTATGGAAGAATAATATATGGATTAGCGCTGCGCCGTACTCTCATCACTATTGGTG |
| | AAGAAATGGTGAATGCCGCTTATGAAGCGTCTCTTGATAAATCCCCTTCAATCCAAAT |
| | TGAAGAAGCAGAACGAAAACTATTTCATTTAGCAGAAAATGGACGATACGATGGGGGA |
| | TTCCATACATTTTCTGATGCAATGACAGTCGCAATTGATATGGCTGGACAAGCGTTTA |
| | ATCGAGATGGAAGACTCGCCGGAGTTTCTACAGGAATTCAAACGCTTGATAAACAAAT |
| | GGGAGGATTACAACGTTCCGATCTCATAATTATTGCGGGACGCCCAGGTATGGGAAAA |
| | ACATCACTGGCAACGAATATTGCCTATAATGTTGCAGATGCATATAAAGCAGAACTAC |
| | AAACAGATGGATCTTATAAAACTATTAATGGTGGCATTGTTGGATTCTATTCTTTGGA |
| | AATGTCTTCTGAACAACTCGCTACCCGCATTATTTCTGAACAAACAGAAGTTCCTTCT |
| | TCAAAGATACGCAGAGGAGAATTGACGCGACCTGACTACGAAAAAATTGTTGCTTGTT |
| | CTCAGGTAATGCAAAAATTACCACTATACATTGATCAAACTGGTGGTATTTCCATGAG |
| | TCAATTAGCGACACGTGCACGTCGATTAAAACGTCAACGAGGACTTGATCTTCTTATT |
| | GTAGACTATATACAACTAATGACTACTTCTAAAAAAATTGAAGAAAATCGTGTTCTGG |
| | AAATCACAGGCATTACCATGGCATTAAAAGCTCTGGCAAAGGAACTTAATATACCTAT |
| | CATTGCGCTTTCACAACTTTCTCGACAAGTAGAGAATCGTGATAATAAACGCCCTCAA |
| | CTTTCTGATTTACGTGAATCTGGTTCCATTGAACAAGATGCAGACGTAGTACTATTCG |
| | TCATCCGCGATGAATATTATATCAGAAACAAAGAACCTACAAACAAAGATGATATTCT |
| | TGCTTATACAAAGTGGCAGGAAGATCTTAAAAGAGTAAAAGGCATTGCCGATATTATT |
| | ATAGCCAAACAACGTCATGGACCAACAGGAACGGTTACCCTTGCTTTTCAAGCAGAAT |
| | TTACACGCTTTTCCGCATTATCTGATTCGTCTTATCAAACTGGTGAACATTTTGATTC |
| | TGCATAA |
| | (SEQ ID NO: 101) |
| >CP003467.1:5671 | ATGATTAAGAATATTTCTTCTATTAATTCAGAATTTATTATAATAAATTATATTTATA |
| 7-57868 | ATGATTTTTATTTTTCTAAAAAAATATTAGAAATTTTAGAAGAAGATGATTTCTTTTT |
| Candidatus | CGACACAACAAAAAAATTTTTTTTACAAAAACAATTTTTTTTAAAATTAAAATTTTTG |
| Carsonella ruddii | GATGAAAAATTAGTTTTTTCTAATCTTAAAAATTTATTAGAAAAATCTAAAAAAAGAA |
| DC, complete | AAATGTTAAAAATATTATTTAACATAAGTTTTGATTTAATATATTATAACAATATTGA |
| genome_DnaB | AGAAAATATATTTGATAAAATAATTTTTTTGCAAAGTTTTAAAAAAAGTAATTTTTTT |
| | TTTAAAAAAATTGATTATAAAAAAATACTAAAAAATTATTTTTTAGAGAATGAAAAAA |
| | ATTTTTTTACAGGATACAGTAAATTAGATAATATTATAAATGGTTTACAAAAAGGAAA |
| | TTTAATAATCTTAGCAGGAAGACCTTCAATAGGAAAAACTTCTTTTTCATTAAATATT |
| | GTTAAGAATTTAATATTATTCAAAAAAAAAATAATTTTCTTTTCTTTAGAAATGACAG |
| | TAAGTCAACTTTTAATAAGATTAATATCTATTATACTAGAAATTAATCACGATAATTT |
| | TGTAAAAAATAATTTTAATAATATAGATATAAAAAAAATTTCTTTTTTCATAAAAAAC |
| | ATAAATTTTGATGAATTAATAATAAAAGACTGTTCAACTCTATCATCTAATGATATAG |
| | AATCGCAACTAGAGTATTATAAAAAGAATAATATTAATATTGAAATAATAATCATTGA |
| | TTATATTCAATTAATGAAATCTGAATTTTATTCAAATAATAGAGTTTTAGAAATATCC |
| | GATATTTCTAGATCATTAAAATTAATTGCAAAACATTTTAATTGTGTTGTAATTGCGC |
| | TATCTCAACTTAATAGATTAGTAGAATATAGAGTTGAAAAAACTCCTGTTTTATCTGA |
| | TTTAAGAGATTCTGGTTCAATTGAACAAGATGCAGATATAGTTATTTTTTTACACAAA |
| | AAAAAATTAAACTTTGTTGATATTTTAGTAGCTAAAAACAGAAATGGTTCTTTAGGAA |
| | TAATAAATTTTATTTTTAAAAATGAATATACTAAATTTTTACAAATTTAA |
| | (SEQ ID NO: 102) |
| >CP002371.1:1022 | TTAAATAAAAATATCTCCTGTAAAAATTTTACGAAAACAACCTTTTTTTTCTTCCAAT |
| 461-1023219 | AATAAATATCCAAAATCATCAACTCCAACAAAATGACCTGAAATAGAAAATTGGGGAA |
| Candidatus | GTTTAATCGTGATAGGATTACCTATTCCGTAAGCAAAACAACGCCAAAGTTTCATGAT |
| Liberibacter | TTCTTCTCGTCCTGTATCTTTTTTCCAAATAGCTAACATTTTTGCAATTTCTTGGAAA |
| solanacearum | AAAATTGGTAAAATACTCTTTAAATCTATACATCCTCCCTCTTTTCTCAGAGAAGTAA |
| CLso-ZC1, | CAGGATAAGAAGTATCTTTGGGACAATACTCAACGTTCAACCCTATTCCAATAACAAT |
| complete genome | TGCTTGTAAACCACTTTTTAAATCTAAAGTCTCAATTAAAATTCCAGCAATCTTACGT |
| GenBank: | TGAAAAACTAACAAATCATTTGGCCATTTTATTTTGACATCTACCTCAGCAGGAAGAA |
| ADR52712.1 | TAGATGCTATAATTGAGTGCATAGCAACAGAAACTGCAAAAGACAAGAGCGTTAGGGA |
| Biotin-(acetyl-CoA | ATCCTTCGATATAGAATCAATCAATAGTAACGAAGCGTATAGATTCCCTTTATCAGAA |
| carboxylase)LIGASE | ACCCATTTTTTCCCTCCACGACCTCGACCAGATGTTTGACGTGATGCAATTATCCATA |
| | AATTTCCTAAATCGCCAGATAATGCACGTTTCATACATTCATCGTTTGTAGAAACGAT |
| | AGTATCAAAAAATTCATACCGAAAATTATAAAGTTTTTTACATTCAGAAAGATCAGAT |
| | AACAT |
| | (SEQ ID NO: 103) |
| >NZ_AP014595.1:8 | ATGTTTTCTAATCTTAGTGAATGTCAAAATCCTTATAGTTTTCGATATGAATTCTTTG |
| 29256-830014 | ATACTATTTCTTCTACTAATGATGAATGTATGAAACGTGCGTTATCTGGAGATTTGGG |
| Candidatus | AAATTTGTGGATCGTTGCGTCTTGTCAAACGGCTGGTCGAGGTCGTAGGGATAATAAA |
| Liberibacter | TGGATTTCTGATAAGGGCAATCTCTATGCCTCGTTGITATTGATTGATTCTATATCAA |
| asiaticus str. Ishi-1 | AAGATTCCCTAACGCTCITATCATTTGCTATTGCTGTTGCTATGCGTTCAGITATAGC |
| DNA, complete | ATCTACACTTCCTGTTGGAACAGATGTTAAAATAAAATGGCCCAATGATATACTAGTC |
| genome -- Biotin- | TTTCAACGTAAAATTGCTGGCATATTAATTGAGACTCTAAATTTAAAAAATGGTTTAC |
| (acetyl-CoA | AAGCAGTTGTCGTTGGAATAGGATTGAACGTAAAGCATTGTCCTGTAGACACCCCTTA |
| carboxylase)- | TCCTGTTACATCTTTGCAGAGAGAAGGAGGATGCATCGATTTAAAGGATATTTTTTCC |
| Ligase | CTTTTATTTCAAGATGTTGCGAGGGTATTAGACCTTTGGAAAAAAGATACGGGACGAG |
| | AAGAAATTATGAATCTCTGGCGTTGTTTTGCGTGTGGTATAGGTGATCTTATTACGAT |
| | TAAACTATCATATGGTTCCATTTTAGGTCGTTTTGTTGGAGTCGACGATTTCGGATAT |
| | CTATTATTAGAAGAAAAAAAAGGATGTGTTCGTCAGATTTTTACAGGAGATATTTTTA |
| | CTTAG |
| | (SEQ ID NO: 104) |
| >CP001677.5:256428- | ATGAAACAGAAAAAGTCGTTAGATGAACGATATATTGAAGATGTCATTCAAAGTTTTC |
| 257060 | AAAAAGGAGAAATGGTTATCGTCACGGATGCAGATGACCGCGAGAATGAAGCTGACTT |
| Candidatus | AGTACTTGCCGCCATACATTGCACATCCGAAAAAATGGCATTTATCATCCGCCATACT |
| Liberibacter | TGTGGAATCGTTTGCACTCCCATGCCCTTTCACACTGCACATAAGCTTAAACTTAATC |
| asiaticus str. psy62. | CTATGGTATTAGAAAATGAATCCGTTCACAAAACTGCTTTTACCGTCTCCGTTGATTC |
| P_012778578.1 3,4- | TAAACATGGAATCACTACTGGCATATCTGCCGATGACAGAGCATATACCATCAAAAAC |
| dihydroxy-2- | CTTGCTAATCCACACAGCATTGCGGATAATTTTGTCCGCCCAGGACATATCTTCCCTC |
| butanone-4- | TTATATCCCGCGATGGAGGAGTACTAGTTCGCCCTGGTCATACAGAAGCATCAGTCGA |
| phosphate synthase | TTTATGTAAAATCACAGGACTACCTCCGATAGCGGTTATTTGCGAATTAGTTAACGAT |
| Ribulose-B | GATGGAACAATCAAAAAAGGAAAACAAGTGATTGAATTTTCAAAGAAATATGACTTGA |
| | AAATCATTTCAGTCCAAGATCTAATTGCTTGGCGAAAGAAAAAAGAAATATAA |
| | (SEQ ID NO: 105) |
| >CP003468.1:2068 | ATGTACAATAAATTTATTTCTTCTACTAAAGAAATTATTTCTGAATTAAAGATTGGAA |
| 67-207982 | ATATGATTATTTTAGTAGATTCAGAAAATCGTGAAAATGAAGGAGATATTATATTATC |
| Candidatus | TTCTGATTTTGTAACACCAAATGCTATTAATTTTATGTCTAAATATGCAAGAGGTTTA |
| Profftella | ATTTGCATGACATTAACAGAAAAACATTGTATTCAGCTCAAATTAGATATGATGACAA |
| armatura, complete | AAAAAAATAATTCTTCTTTTGGTACTAATTTTACCGTTTCAATTGAAGCGGCAAATGG |
| genome | AGTCACAACGGGAATTTCGGCATCAGATCGAGCACATACAATTAAAGTTGCTTCATCA |
| | AAAAAAGCTAAACCTTCTGATATCGTCCAACCTGGACACATTTTTCCTCTACAAGCTA |
| | AAAAAGGAGGAGTATTAATACGAGGAGGTCATACTGAAGCTGGTTGTGATTTAACAAA |
| | ATTAGCTGGACTAACACCATCTGCAGTAATTTGTGAAATTTTAAATGATGATGGAACT |
| | ATGGCACGTTTACCAGATCTAATAAAATTCTCTAAACTACATAATATTAAAATTGGAA |
| | CTATTTCTAATTTAATATGTTATCGTTATAAATATGAAAATATTATTAAAAGAAAAGC |
| | GGAATATAATATAAATACTTTATATGGAAATTTTAAAATCATTGTGTATATAGATAAA |
| | ATCAATAATGACATTCATTTAACTTTAATATATGGCTCAATCAATCCTAATAAAATAA |
| | TACCAATATGGCTAAATCAACAACTTTCAATATTTGATTTTTTACACGCCAAAACAAG |
| | CTCATGCACACAAAATAATATTTCTTCAATAATGAAAATAATGAAATTAATTAAAAAT |
| | GGTATTTTAATTTTAGTTAATTGTGAATATTCTATAAATTCTATATTTAATCAATTAA |
| | AAAATTTTAATATAATTGACGAAAAAAATAATATTATTCAACCAAAAGATCATAAAGA |
| | TACTAATTTAGTTCATATTAATAATATTAATGCGCAAATATTAAAAGATCTTAATATC |
| | AAAAAAGTTAAATTAATGACGCATTTAAATGATATTTCACCTTTTTTAAATTTTAATA |
| | TAAAAATAAAATAA |
| | (SEQ ID NO: 106) |
| >CP002371.1:4615 | TTATACTTTTTTTTGTTGACGCCAAGCAATTAAATCTTTGATTGAAATAATTTTTAAT |
| 01-462133 | CCATGTTTTATTGAAAATTCAATGACTTGCTTTCCTTTTTTGATTGTTCCATCATCAT |
| Candidatus | TGACTAGTTCACAAATAACCGCTACTTGGGGCAGTCCTGCAATTTTACATAAGTCGAC |
| Liberibacter | AGATGCCTCTGTATGACCAGGACGGATTAGCACTCCTCCATCACGAGAGATGAGGGGA |
| solanacearum | AAGATGTGTCCTGGACGGACAAAATCATTTGCAGTGCTTTGTGGATTAGTAAGATTTT |
| CLso-ZC1, | TGACAGTATGTGTCCTATCAGCAGAAGATATACCTGTAGTAATACCATGTTTAGAGTC |
| complete | AACGGATACAGTAAAAGCAGTTTTATGGACTGATTCGTTTTCTAATACCATAGGATTA |
| genome_dna | AGTCCGAGTTTACGTGCATTTTGGGAAGGCATGGGTGTGCAAACGATTCCACAGGTAT |
| | GGCGAATAATAAACGCCATTTTATCGGACGTGCAATGTATAGCTGGAAGTACTAAATC |
| | AGCTTCATTTTCACGATCATCTTCATCTGTCACGATAACCATGTTACCTTTTTGAAAA |
| | TTCTGGATTACATTTGAAATATGTTGTTCATTCAATGGCATTTTTTGTTCCAT |
| | (SEQ ID NO: 107) |
| >Dcitr18833.1, | ATGGCAGAAATGCGACAAGAGTGTAAATGTCACGGCATGTCTGGATCCTGTACTGTCA |
| www.CitrusGreening.org/ | AGACCTGCTGGATGAGGCTTCCTAACTTCAGAGTTATTGGAGATGCTCTCAAAGATCG |
| Diaphorina/OGSv1.0 | GTTTGACGGCGCGTCCAGAGTGATGGTCAGTAATGAAGGTAACGTTCGTGGCAGCAGT |
| | GTGGGGAAGAAAAGTCGGTACAACTTCCAGCTGAATCCGTACAATCCTGAACACAAAC |
| | CTCCAGGTGTGAAGGATTTGGTCTACCTGGAAACCTCGCCAGGATTTTGTGAGAAGAA |
| | TCCCGCACTGGGAATACAGGGCACGCATGGACGACAATGCAATGACACCAGTATTGGT |
| | GTAGATGGTTGTGATCTGATGTGCTGTGGGCGAGGCTACAGAACACAGGAGATCACCG |
| | TCGTGGAACGGTGTGCTTGIGCTTTTATTTGGTGCTGTGAGGTCAAATGTAAAACATG |
| | TAGAACAAAGAAAACCATTCACACTTGCTTGTAGATAATCATTATTATTAATATTATT |
| | AGTCTAAGAGAATGATTCCAGAACTGTGCCTATTGATCCATACAATTACAATTGTACG |
| | CTGTATGGTGTTGTTCAGAAAGATAATCTATATAGACTTGCAAGGACCTACCTACATT |
| | ATAGCTATGTTAGTACTGAGGTAATTGCCATACAACGATATACATAGATCAGTACAAG |
| | TCTAAAGAGTAAATCGGCCTTACTGAAATAGCAGAATATTTGCTACAGGACTTGGATA |
| | AATTCGTATGAAGTTATGAACGTAAATGTATCTCTTTCTTGCACTTTTGCTACACCTT |
| | CAACATGAAAATCGGACAACATTGGAAAAATTCTCGATACTTAAT |
| | (SEQ ID NO: 108) |
| >XM_008486571.2: | ATGGTCCTCAATGTTTCTCTGTTACAGCATGTGATGGCAGAAATGCGACAAGAGTGTA |
| 182-712 | AATGTCACGGCATGTCTGGATCCTGTACTGTCAAGACCTGCTGGATGAGGCTTCCTAA |
| Diaphorina citri | CTTCAGAGTTATTGGAGATGCTCTCAAAGATCGGITTGACGGCGCGTCCAGAGTGATG |
| wingless | GTCAGTAATGAAGGTAACGTTCGTGGCAGCAGTGTGGGGAAGAAAAGTCGGTACAACT |
| LOC103521460), | TCCAGCTGAATCCGTACAATCCTGAACACAAACCTCCAGGTGTGAAGGATTTGGTCTA |
| mRNA | CCTGGAAACCTCGCCAGGATTTTGTGAGAAGAATCCCGCACTGGGAATACAGGGCACG |
| | CATGGACGACAATGCAATGACACCAGTATTGGTGTAGATGGTTGTGATCTGATGTGCT |
| | GTGGGCGAGGCTACAGAACACAGGAGATCACCGTCGTGGAACGGTGTGCTTGTGCTTT |
| | TATTTGGTGCTGTGAGGTCAAATGTAAAACATGTAGAACAAAGAAAACCATTCACACT |
| | TGCTTGTAG |

### Example 2

Transfection of FANA ASOs into Cultured Insect Cells

To assess the ability of FANA ASOs to penetrate insect cells, a suspension of S2-wDi cells was incubated with a fluorescently labeled FANA oligo for 24 h. An aliquot was added to a coverslip coated with the lectin concanavalin A, which induced the cells to spread and adhere to the glass (Buster et al., 2010). The cells were fixed, mounted and finally imaged. Confocal microscopy revealed that the oligo could be readily detected inside S2-wDi cells, within one day post-treatment and without using any transfection agent. No signal was detected in untreated S2-wDi cells.

### Example 3

### Localization of FANA ASOs in D. Citri Alimentary Canal

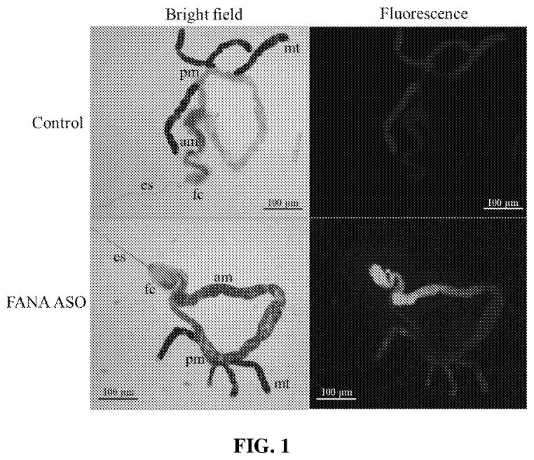

The capability of D. citri adults to ingest FANA ASOs from the vascular system of Citrus plants was evaluated. Adult psyllids were allowed to feed on ‘Pineapple’ sweet orange leaves, which had previously absorbed a solution containing a fluorophore-tagged FANA oligo control [SEQ ID NO: 1 and SEQ ID NO: 2]. After 48 h the psyllids alimentary canals were dissected and imaged. Fluorescent microscopy demonstrated that the FANA ASO was delivered to the insects through the Citrus leaves vascular tissues; the oligo was detected mainly in the psyllid esophagus, filter chamber and anterior midgut (FIG. 1).

### Example 4

Silencing of wDi gyrA in Cell Culture

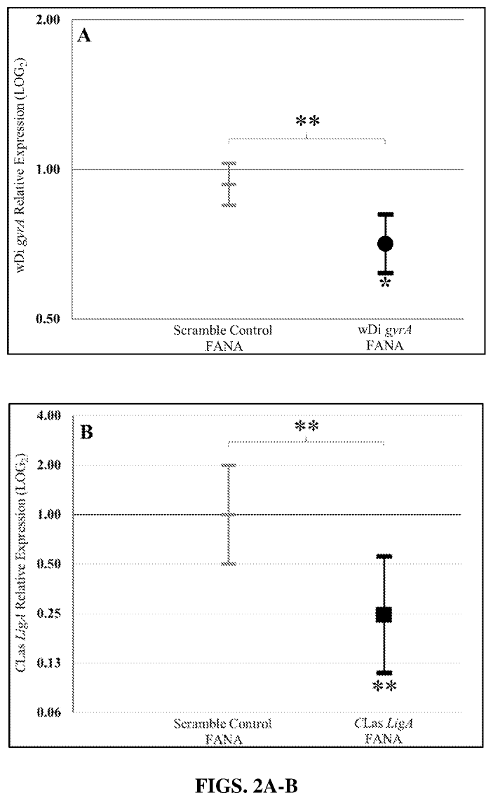

To examine the competence of FANA ASOs to mediate the degradation of specific bacterial genes in cultured insect cells, S2-wDi cells were incubated with 5 μM of a FANA oligo complementary to the mRNA of Wolbachia-Diaphorina, wDi gyrA gene (gyrA-FANA) [SEQ ID NO: 38]. As a negative control, a group of cells were exposed to a FANA oligo designed with a scramble sequence not targeting any wDi gene (scramble control FANA) [SEQ ID NO: 4]. Total RNA was extracted seven days post-treatment and analyzed by qRT-PCR, using primers specific to the wDi gyrA gene. Compared to untreated cells, the gyrA-FANA treatment resulted in a significant 30% reduction in the amount of the target mRNA [t (16)=−2.60, p=0.019] (FIG. 2A). The wDi gyrA transcript level was significantly lower in the cells exposed to gyrA-FANA, compared to cells treated with the scramble control [t (16)=2.98, p=0.009]. No significant difference in the expression of the wDi gyrA gene was observed between cells incubated with 5 μM of the scramble control FANA and untreated cells [t (16)=−0.65, p=0.52].

### Example 5

### Silencing of CLas Ligase-A in D. Citri

The competence of FANA ASOs in silencing bacterial genes inside D. citri was evaluated. A FANA antisense oligo was designed to target the CLas NAD-dependent DNA Ligase A gene [SEQ ID NO: 71] (LigA-FANA), by making it complementary to its mRNA. The oligo was delivered to D. citri adults through the vascular system of excised Citrus leaves, that had taken up a 5 μM FANA solution. The scramble control FANA was used as a non-target control. Seven days post-treatment, total RNA was extracted from the psyllids and analyzed by qRT-PCR, using primers specific to the CLas LigA gene. Feeding D. citri adults with LigA-FANA significantly decreased the expression of the bacterial target gene by 75%, when compared to untreated psyllids [t (16)=−3.18, p=0.006] (FIG. 2B). The target transcript level was significantly lower in the psyllids exposed to LigA-FANA, compared to insects treated with the scramble control [t (13)=3.08, p=0.009]. There was no significant difference in the amount of CLas LygA mRNA, between psyllids that were fed the scramble control FANA and untreated psyllids [t (13)=−0.007, p=0.99].

### Example 6

Effect of gyrA-FANA on the Viability of wDi Cells

wDi was isolated from Drosophila S2 cells and incubated with gyrA-FANA, [SEQ ID NO: 38] for four days. The cells were collected and treated with propidium monoazide (PMA) before quantifying the bacterial copy number by qPCR. Propidium monoazide is a membrane-impermeant dye that selectively penetrates cells with compromised membranes (dead cells). PMA covalently links to DNA, which inhibits PCR amplification, (Nocker et al., 2007); therefore, only DNA from live cells was amplified. The number of viable wDi cells was greatly reduced in the [SEQ ID NO: 38] Wolb-gyrA-FANA treatment, as compared with wDi cells that were untreated or incubated with the scramble control FANA [SEQ ID NO: 4] (FIG. 3). This result indicated that decreasing the expression of the DNA gyrase subunit A gene seemed to be lethal for the Wolbachia cells. The high wDi copy number observed in the scramble control treatments demonstrated that the FANA chemistry was not toxic to the bacterial cells or the Drosophila S2 cells (SEQ ID NOS: 42, 43, 44 and 45).

### Example 7

### Effect of Lig-A-FANA on CLas Quantification in D. CITRI and Citrus PLANTS

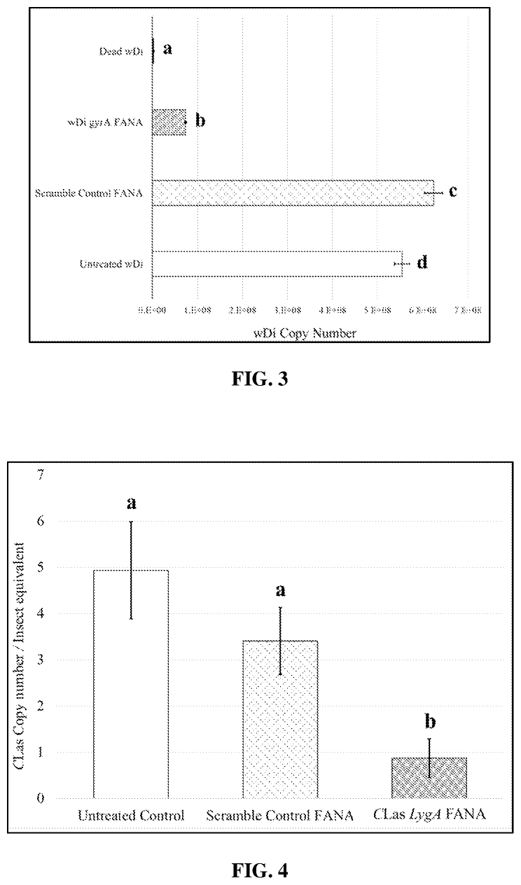

D. citri adults were fed an artificial diet containing NAD-dependent DNA ligase, LigA-FANA, [SEQ ID NO: 71, or SEQ ID NOs: 72-74] at a concentation that significantly reduced the expression of the CLas LigA gene. Seven days post-treatment, quantitative PCR was used to estimate CLas copy number in the psyllids, by targeting the bacterium 16S rRNA gene. CLas density was significantly lower in the psyllids that ingested LigA-FANA, compared to untreated psyllids or insects fed with the scramble control-FANA [SEQ ID NO: 4] (FIG. 4). No difference in CLas copy number was observed between D. citri adults exposed to the scramble control and untreated psyllids. The results showed that degrading the CLas LigA mRNA was detrimental for the bacterium density.

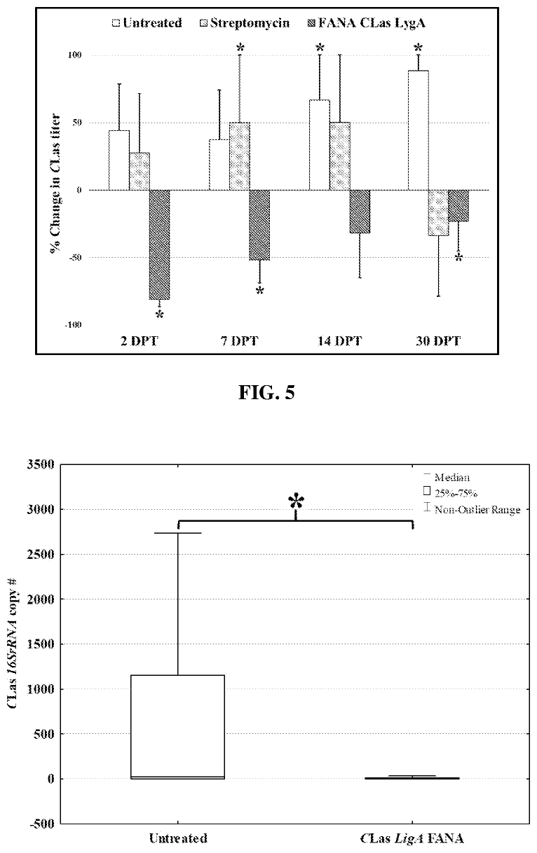

Delivering DNA ligase, LigA FANA [SEQ ID NO: 71] through root infusion to CLas-infected Citrus trees, significantly reduced the quantity of the bacterium (FIG. 5). The supression effect was persistent during the 30 days sampling period, being the highest Liberibacter repression observed two and seven days post treatment (81% and 51.6%, respectively). Root infusion application of streptomycin sulfate into infected Citrus plants did not significantly reduced the Liberibacter titer (FIG. 5). Citrus plants infused with water significantly increased their CLas quantity by 67% and 89% after 14 and 30 days, respectively (FIG. 5).

### Inoculation Efficiency Assays

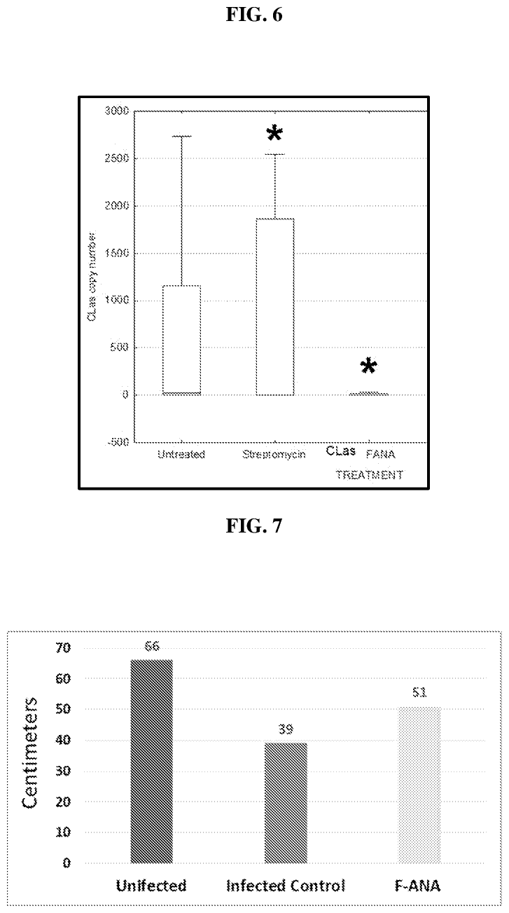

To test the capacity of FANA ASOs in reducing CLas transmission, infected psyllids were treated with CLas LigA-FANA. Infected adult psyllids (≤3 days old) were placed in feeding arenas where they were exposed to a FANA ASO solution or a 17% (w:v) sucrose solution. Seven days post-treatment the psyllids were transferred to uninfected Citrus macrophylla seedlings, six to nine months old, for an inoculation access period where they were allowed to feed of 15 days. Twenty adult psyllids (10 males and 10 females) were caged with individual seedlings. Five plants were used per treatment. CLas-free psyllids were caged with healthy Citrus macrophylla seedlings as negative control. After the inoculation access period, every psyllid developmental stage was removed from the seedlings and the adults were collected in 80% ethanol for subsequent qPCR bacterial detection. The seedlings were sprayed with insecticides (to eliminate any remaining psyllid) and moved to a greenhouse. Three months later, the CLas copy number of each seedling was quantified by collecting three leaves and detecting the titer of the bacterium as described before. As illustrated in FIGS. 6, Psyllids that were treated with LigA-FANA were less successful in transmitting the pathogen into citrus plants, compared to untreated insects (U=720, n1=n2=45, p=0.018), as observed in the significantly lower CLas copy number detected in the citrus plants that were exposed to the FANA-treated psyllids (FIG. 7). Kruskal-Wallis H test and Mann-Whitney U test were used to compare the plant's CLas copy number between the treatments.

### Example 8

### Effect of Gyrase-a, GyrA-Fana Treatment on CLso Infected Potato

Candidatus Liberibacter solanacearum” (CLso) is an economically important pathogen of Solanaceous crops and the putative causal agent of zebra chip disease of potato (Solanum tuberosum L.). This pathogen is transmitted to Solanaceous species by the potato psyllid, Bactericera cockerelli (Sŭlc). (Sengoda et al, 2014; Crosslin et al, 2011; Munyaneza, J. E. 2015).

FANA treatments of CLso-infected potato plants included (scramble F-ANA blank Control [SEQ ID NO: 3] infected plants, and CLso GyrA-FANA's, [SEQ ID NOs: 15, 16, 17, 18, 19 and SEQ ID NOs: 75, 76, 77,] and [SEQ ID NO: 46-70] CLso-DnaB-FANA's and were compared to uninfected potato for growth and symptom severity. Five potted potato plants (Russet Burbank cultivar) in one gallon pots (five per treatment) were inoculated by psyllid transmission with the Zebra chip pathogen (haplotype ‘B’) by confining three Liberibacter-infected psyllids to a single leaf for one week. Control plants were not challenged with potato psyllids (B. cockerelli). Following a 1-week inoculation access period, the psyllids were removed and confirmed to harbor the zebra chip pathogen using PCR (Crosslin et al. 2011). Three leaflets from three separate leaves will also be collected from each plant to quantify titers of the zebra chip pathogen using quantitative PCR (Sengoda et al. 2014). Each plant was then treated with 100 mM FANA solution (one of five target sequences or non-target control) or water (untreated controls) using a 100 ml solutions as soil applied drench. Two weeks (-14 days) following the initial treatment, second doses of treatments were applied in the same manner as described. At that time three leaflets from separate leaves were collected from each plant to quantify pathogen titers. Each plant height was measured every two weeks post treatments for growth (FIG. 8).

CLso infected Potato plants treated with CLso-GyrA-FANA's, [SEQ ID NOs: 16, 17, 18, 19] as a soil applied drench to soil in one gallon potted plants, resulted in improved growth (ave. 51 centimeters height) compared to CLso infected untreated control plants (ave. 39 centimeters height), while uninfected healthy potatoes average 66 cm in height (FIG. 8). F-ANA Treated CLso-infected plants had greater growth than untreated CLso-infected controls.

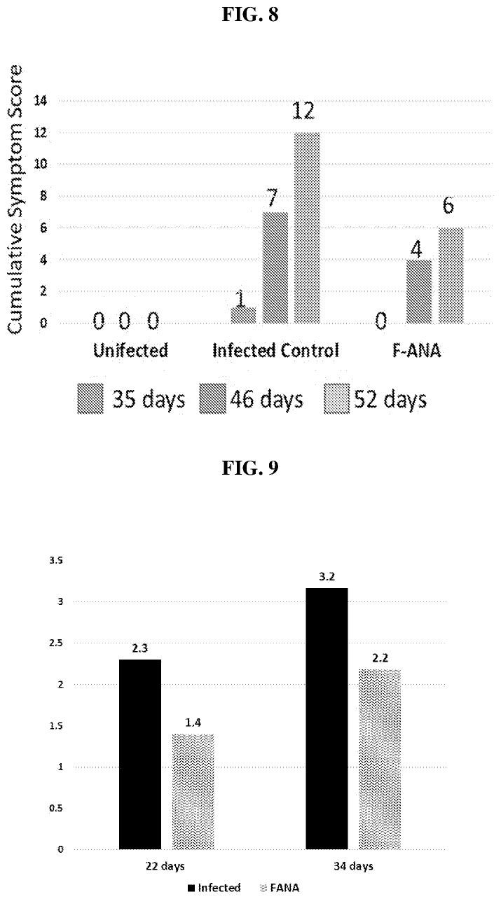

Development of foliar symptoms was monitored and photo-documented each every 2 weeks, and was ranked from 0 (no symptoms) to 3 (severe). Foliar symptom scores were summed for all plants in each treatment at three timepoints post inoculation by psyllids, for timepoints on days 35, 46, 52 (FIG. 9).

Foliar Symptom Scores resulted in a total summed score that was half that of the symptom severity in the untreated CLso infected controls (FIG. 9). The F-ANA treated plants had significant reduction of symptoms (score 6), being about half that of the untreated CLso-infected control treatment (score 12). Uninfected Scrambled F-ANA negative control, CLso-infected no treatment Control, and CLso-infected, F-ANA treated. Visual Foliar Symptom Scores ranged from 0 to 3, with three being most severe symptoms, zero no symptoms.

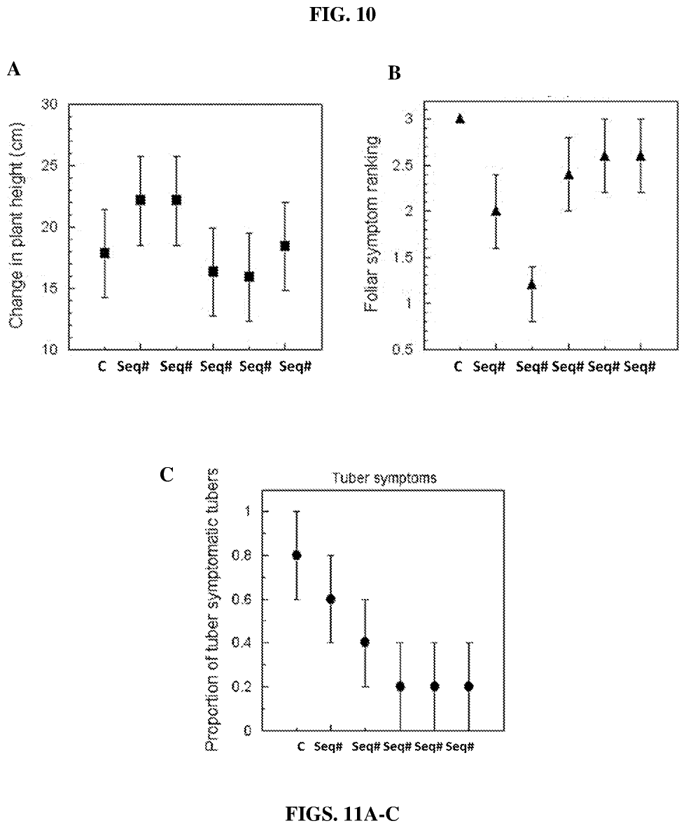

In general CLso titers were numerically reduced in treated plants compared with CLso infected control two weeks after treatment (FIG. 10).

As illustrated in FIGS. 11A-C, in an 8 week trial, CLso infected potato plants were treated with control and F-ANA products applied to the soil, and potato tuber disease symptoms were evaluated. [SEQ ID NOs: 15,16, 17, 18, 19] or [SEQ ID NOs: 64, 65, 66, 67, 68, 69, 70] and [SEQ ID NOs: 75, 76, 77].

Tuber disease symptoms evaluation included the attribution of a symptom score:
0=none,
1=two to three leaves slight yellow,
2=five or more leaves yellowing,
3=most leaves yellow, with curling;
and the assessment of tuber symptoms, which is the browning aspect observed when a potato is sliced into chips and fried in oil (the browning is due to excess starches and sugars post infection by bacteria).

There was no significant difference observed in plant growth between the control plants and treated-infected plants at eight weeks. Foliar disease symptoms were significantly reduced by all products (F-ASO) [SEQ ID NO: 15, 16, 17, 18, 19; 64, 65, 66, 67, 68, 69, 70; 75, 76, 77], with the most symptom reduction observed from treatments with CLsoDNAB-1[SEQ ID NO: 64] and Gyrase-Ext-2 [SEQ ID NO:18]; as observed by the reduction in foliar symptom ranking. Tuber disease symptoms were significantly reduced compared to CLso infected Control tubers, in treatments with F-ANA Products: CLso-DNAB-2 [SEQ ID NO: 65] and DNAB4, [SEQ ID NO:67] and CLso-2Gyr-EXT-4, [SEQ ID NO:19] as observed by the reduction in the proportion of symptomatic tubers.

Although the invention has been described with reference to the above examples, it will be understood that modifications and variations are encompassed within the spirit and scope of the invention. Accordingly, the invention is limited only by the following claims.

1. A method of decreasing target RNA or DNA expression in a plant-chewing or piercing-sucking plant-feeding insect, comprising providing an antisense oligonucleotide to the insect, or a member of its microbiome, wherein the oligonucleotide comprises at least one 2′-deoxy-2′-fluoroarabinonucleotide (2′F-ANA)-modified nucleotide and at least one 2′-deoxyribonucleotide.

2. The method of claim 1, wherein the target RNA is insect RNA, viral RNA, or bacterial RNA, and wherein the bacterial RNA is DNA Gyrase, subunit-A; NAD-dependent DNA Ligase-A, DnaB-Helicase C terminal domain, D-Ribulose A (riboflavin), Ribulose A, Ribulose B (3,4-dihydroxy-2-butanone-4-phosphate synthase), Acetoacetyl-[acyl-carrier protein], Acetylcholinesterase, Amidophosphoribosyltransferase, Acetyl-CoA carboxylase biotin carboxylase subunit, Biotin Ligase, Biotin-(acetyl-CoA carboxylase)-Ligase, Malonyl Coenzyme A, wsp, or 16SrRNA, thereby decreasing target RNA expression.

3. The method of claim 1, wherein the bacterial RNA is from Candidatus Liberibacter asiaticus (CLas) or from Candidatus Liberibacter solanacearum (CLso).

4. The method of claim 1, wherein the insect is Diaphorina citri (D. citri) or Bactericera cockerelli.

5. The method of claim 1, wherein the oligonucleotide is selected from the group consisting of SEQ ID NOs: 5-77.

6. The method of claim 5, wherein 2′F-ANA modified nucleotides are positioned within the oligonucleotide according to any of Formula 1-16.

7. The method of claim 1, wherein the insect feeds from plant vascular (xylem or phloem), mesophyll, root, flower, fruit, seed, or leave.

8. The method of claim 1, wherein the insect ingests the oligonucleotide by consuming plant material containing the oligonucleotide.

9. The method of claim 8, wherein the plant material comprises leaf tissue, root tissue, stem tissue, flower tissue, fruit, sap, xylem, phloem, mesophyll or a combination thereof.

10. The method of claim 8, wherein the plant is a citrus plant or a Solanaceous plant.

11. The method of claim 10, wherein the citrus plant is selected from the group consisting of orange, lemon, clementine, lime, grapefruit, pomelo, citron, mandarin, tangelo, trifoliate, rootstock and ornamental citrus relative.

12. The method of claim 10, wherein the Solanaceous plant is selected from the group consisting of nightshade, potato, tomato, tomatillo, eggplant, bell pepper and capsicum pepper.

13. The method of claim 8, wherein the oligonucleotide is:
(i) applied to a plant by topical foliar spray, soil applied solution, or irrigation; and/or
(ii) bound to an absorbent, bound to a peptide, or bound to a synthetic or natural gel, agar, clay, cellulose, or other material,
for application, spreading, mixing with soil, and/or for being applied to soil in solution or in water for root uptake or root soaking.

14. The method of claim 1, wherein the oligonucleotide sequence is a complement to the sequence of the target insect or bacterial RNA, and wherein the oligonucleotide sequence has at least 80%, 85%, 90%, 95%, 98%, 99%, or more sequence identity to the complementary sequence of the target RNA.

15. The method of claim 1, wherein the target RNA or DNA is prokaryotic or eukaryotic.

16. The method of claim 1, wherein the target RNA or DNA is selected from the group consisting of messenger RNA (mRNA), microRNA (miRNA), small interfering RNA (siRNA), antisense RNA (aRNA), short hairpin RNA (shRNA), transfer RNA (tRNA), ribosomal RNA (rRNA),small nuclear RNA (snRNA), double-stranded RNA (dsRNA), locked nucleic acid (LNA), transfer-messenger RNA (tmRNA), viral RNA, viral DNA, Polynucleic acids circular ssDNA, and circular DNA.

17. The method of claim 1, wherein the oligonucleotide sequence has at least 80%, 85%, 90%, 95%, 98%, 99%, or more sequence identity to the complementary RNA or DNA sequence.

18. A method of inhibiting bacterial growth in a plant-chewing or piercing-sucking plant-feeding insect comprising providing an antisense oligonucleotide to the insect or to its microbiome,
wherein the oligonucleotide comprises at least one 2′-deoxy-2′-fluoroarabinonucleotide (2′F-ANA)-modified nucleotide and at least one 2′-deoxyribonucleotide, wherein the oligonucleotide binds to a target RNA or DNA,
wherein the target RNA is important for bacterial replication or survival, and
wherein the target is DNA Gyrase subunit-A, NAD-dependent DNA Ligase-A, DnaB Helicase C terminal domain, D-Ribulose A (riboflavin), Ribulose A, Ribulose B (3,4-dihydroxy-2-butanone-4-phosphate synthase), Acetoacetyl-[acyl-carrier protein], Acetylcholinesterase, Amidophosphoribosyltransferase, Acetyl-CoA carboxylase biotin carboxylase subunit, Biotin Ligase, Biotin-(acetyl-CoA carboxylase)-Ligase, Malonyl Coenzyme A, wsp, or 16SrRNA,
thereby inhibiting bacterial growth.

19. The method of claim 18, wherein the RNA is from gram negative or gram positive bacteria, wherein the RNA is from E. coli or from Serratia marcescens, or insect endosymbionts, wherein the RNA is from Carsonella rudii, or Profftella armigera, or Wolbachia-Diaphorina, or Wolbachia-Drosophila, or Candidatus Liberibacter asiaticus (CLas) or from Candidatus Liberibacter solanacearum (CLso).

20. The method of claim 18, wherein the insect is Diaphorina citri (D. citri) or Bactericera cockerelli, and wherein the oligonucleotide is selected from the group consisting of SEQ ID NOs: 5-77.

21. (canceled)

22. (canceled)

23. (canceled)

24. (canceled)

25. (canceled)

26. (canceled)

27. (canceled)

28. A method of preventing and/or managing of huanglongbing disease in a citrus tree or plant or preventing and/or managing a Liberibacter zebra chip disease in Solanaceous plant comprising providing an antisense oligonucleotide to a plant chewing or piercing-sucking plant-feeding insect or to its microbiome, wherein the oligonucleotide binds to a target RNA or DNA,
wherein the oligonucleotide comprises at least one 2′-deoxy-2′-fluoroarabinonucleotide (2′F-ANA)-modified nucleotide and at least one 2′-deoxyribonucleotide, and wherein the target RNA is DNA Gyrase subunit-A, NAD-dependent DNA Ligase-A, DnaB-Helicase C terminal domain, D-Ribulose A (riboflavin), Ribulose A, Ribulose B (3,4-dihydroxy-2-butanone-4-phosphate synthase), Acetoacetyl-[acyl-carrier protein], Acetylcholinesterase, Amidophosphoribosyltransferase, Acetyl-CoA carboxylase biotin carboxylase subunit, Biotin Ligase, Biotin-(acetyl-CoA carboxylase)-Ligase, Malonyl Coenzyme A, wsp, or 16SrRNA,
thereby improving plant survival, or yield, or preventing and/or managing huanglongbing disease.

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

48. (canceled)

46. (canceled)

47. (canceled)

48. (canceled)

49. (canceled)

50. (canceled)

51. (canceled)

52. (canceled)

53. (canceled)
